(19) United States
(12) Patent Application Publication
(10) Pub. No.: US 2023/0381182 A1
(43) Pub. Date: Nov. 30, 2023

(54) Biomarkers Related to Immune Checkpoint Inhibitor Therapy and Methods of Using the Same

(71) Applicant: Senda Biosciences, Inc., Cambridge, MA (US)

(72) Inventors: Devin Forest Reed Doud, Arlington, MA (US); Francesca Barone, Solihull West Midlands (GB); Mark Umbarger, Lexington, MA (US)

(21) Appl. No.: 18/248,123

(22) PCT Filed: Oct. 13, 2021
(86) PCT No.: PCT/US2021/054698

§ 371 Date: Apr. 6, 2023

Related U.S. Application Data
(60) Provisional application No. 63/091,192, filed on Oct. 13, 2020.
(60) Provisional application No. 63/107,309, filed on Oct. 29, 2020.

(51) Int. Cl. A61K 31/519; G01N 33/574; G01N 33/68; A61K 39/395; A61P 35/00

(52) U.S. Cl. CPC A61K 31/519; G01N 33/57484; G01N 33/6851; G01N 33/6812; A61K 39/3955; A61P 35/00; G01N 2500/10

(57) ABSTRACT

The disclosure relates to biomarkers predictive of a patient's cancer responsiveness to an immune checkpoint inhibitor therapy. The disclosure provides, in some embodiments, diagnostic and/or prognostic methods of using such biomarkers for determining whether a cancer patient would benefit from being treated with an aryl hydrocarbon receptor antagonist. In some embodiments, the biomarkers described herein may be used to inform and provide effective therapeutic methods for treating cancer comprising administering an aryl hydrocarbon receptor antagonist, optionally in combination with an immune checkpoint inhibitor therapy.

This application claims the benefit of priority of U.S. Provisional Patent Application No. 63/091,192, filed Oct. 13, 2020, and U.S. Provisional Patent Application No. 63/107,309, filed Oct. 29, 2020, the contents of each of which are incorporated by reference herein in their entirety.

The disclosure relates to biomarkers predictive of a patient's cancer responsiveness to an immune checkpoint inhibitor (ICI) therapy. In some embodiments, the disclosure provides diagnostic and/or prognostic methods of using such biomarkers comprising (a) measuring or having measured an amount of at least one biomarker from a serum, plasma, or fecal sample obtained from a cancer patient, (b) comparing or having compared the amount of the at least one biomarker to a threshold score, and (c) determining that the cancer patient would benefit from being treated with an aryl hydrocarbon receptor (AhR) antagonist if the amount of the at least one biomarker is above the threshold score. Aspects of the disclosure further provide methods of treating cancer comprising administering an AhR antagonist, optionally in combination with an ICI therapy.

ICI therapies represent standard of care treatment in the first line setting of several tumors (Gandhi et al., N Engl J Med., 2018, 378(22), 2078-2092; Socinski et al. N Engl J Med., 2018, 378(24), 2288-2301; Antonia et al., N Engl J Med., 2017, 377(20), 1919-1929; Wolchok et al., N Engl J Med., 2017, 377(14), 1345-1356; Cella et al., Lancet Oncol., 2019, 20(2), 297-310). Treatment with this class of therapeutics aims to enhance the immune response of the host to the different progression stages of tumors, with fewer off-target outcomes compared with chemotherapy drugs (Tsai et al., Journal of biomedical science, 2017, 24(1), 35). Major ICI targets include programmed death 1 (PD-1), programmed death-ligand 1 (PD-L1), cytotoxic T lymphocyte antigen 4 (CTLA-4), lymphocyte activation gene-3 (LAG-3), T-cell immunoglobulin and mucin domain-containing-3 (TIM-3), T-cell immunoglobulin and ITIM domain (TIGIT), and V-domain Ig suppressor of T-cell activation (VISTA).

Although ICI therapies have demonstrated significant clinical benefits for treating multiple solid tumors, a lack of response and/or development of resistance to this therapy is observed in many patients (Horsman et al., Int J Mol Sci., 2020, 21(13), 4778). Furthermore, ICI therapies are generally expensive and may provoke inflammatory toxicities in these patients (Das et al., J Immunother Cancer., 2019, 7(1), 306). Therefore, it is important to define the probability of response to ICI therapies in order to design therapeutic strategies able to overcome primary or secondary resistance to such treatments.

Microbial metabolites play a role in patient response to ICI therapies. The gut microbiome is a collection of diverse microorganisms that normally reside within specific areas of the gastrointestinal tract (Dieterich et al., Med Sci (Basel), 2018, 6(4):116; Donaldson et al., Nat Rev Microbiol., 2016, 14(1):20-32). In recent years, the relationship between the gut microbiome and overall host health has been an intense area of medical research (Lin et al., BMC Immunol., 2017, 18(1):2). In addition to playing a role in intestinal barrier homeostasis, the gut microbiota also plays a role in regulating immune functions, with correlations established between dysbiosis of gut microbiota and certain inflammatory and malignant disease states of the gastrointestinal system. The gut microbiota modulates immune response at different levels, influencing neutrophil migration, shaping T cell differentiation in Th1, Th2, and Th17 or regulatory T cells (Tregs), and regulating immune homeostasis and inflammation (Rossi et al., Int Rev Immunol., 2013, 32(5-6):471-474; Pandiyan et al., Front Immunol., 2019, 10:426).

Response to ICI therapy is impaired in response to alteration in the microbiome. Germ free and antibiotics treated mice bearing solid tumors display abrogated response to ICI therapy often associated with decreased cellular immune response and reduced cytokine production (Gopalakrishnan et al., Science, 2018, 359(6371):97-103). Administration of a probiotic cocktail of Bifidobacterium was able to reinstate a more efficient response to ICI therapies in mouse cancer models (Matson et al., Science, 2018, 359(6371):104-108). Stool metagenomic analysis performed in patients with melanoma unveiled different microbiome compositions in responders vs. non-responders to ICI therapies, with responders presenting an abundance of Bifidobacterium longum, Collinsella aerofaciens, and Enterococcus faecium (Matson et al., Science, 2018, 359(6371):104-108). Similarly, microbiota composition analysis in humans (in particular enrichment of Akkermansia muciniphila and Alistipes) has been used to predict the ability to respond to anti-PD-1/PD-L1 therapy in solid epithelial tumors such as non-small cell lang cancer (Routy et al., Science, 2018, 359(6371):91-97). High abundance of Clostridiales, Ruminococcaceae, or Faecalibacterium bacteroidales has been associated with better response to anti-PD-1 via activation of CD4⁺ and CD8⁺ T cells and regulation of Tregs and myeloid-derived suppressor cells in melanoma patients (Gopalakrishnan et al., Science, 2018, 359(6371):97-103). Fecal microbiota transplantation from responder patients into germ-free (GF) mice improved ICI efficiency.

Therefore, preclinical studies in mice as well as early observational human cohorts suggest a complex interaction between bacteria and immune host response in anti-tumor activity. There remains a need to identify biomarkers, for example, microbial metabolites, that are predictive of cancer patients' responsiveness to ICI therapies. Such biomarkers could provide valuable information for determining the effectiveness of ICI therapies and for incorporating such determinations into improved therapeutic strategies for cancer treatment.

The disclosure relates to biomarkers predictive of a patient's cancer responsiveness to an ICI therapy. In some embodiments, the disclosure provides methods of determining whether a cancer patient would benefit from being treated with an AhR antagonist. In some embodiments, the disclosure provides methods of predicting a patient's cancer responsiveness to ICI therapy. Aspects of the disclosure further provide improved methods of treating a cancer patient comprising administering an AhR antagonist to the patient, optionally in combination with an ICI therapy. In some embodiments, the patient has been previously treated with an ICI therapy but is not responding to the ICI therapy. In some embodiments, the patient has been previously treated with an ICI therapy and has developed resistance to the ICI therapy.

Certain embodiments of the present disclosure are summarized in the following paragraphs. This list is only exemplary and not exhaustive of all of the embodiments provided by the disclosure.

Embodiment 1. A method of treating cancer in a patient in need thereof, comprising: (a) measuring or having measured an amount of at least one aryl hydrocarbon receptor (AhR) ligand from a serum, plasma, or fecal sample obtained from the patient, (b) comparing or having compared the amount of the at least one AhR ligand to a threshold score, (c) determining or having determined that the patient would benefit from being treated with an AhR antagonist if the amount of the at least one AhR ligand is above the threshold score, and (d) treating the patient determined to benefit in (c) with an effective amount of the AhR antagonist.

Embodiment 2. The method of embodiment 1, wherein the patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

Embodiment 3. The method of embodiment 1 or embodiment 2, wherein the patient has been treated with an immune-checkpoint inhibitor (ICI) therapy but is not responding to the ICI therapy.

Embodiment 4. The method of embodiment 1 or embodiment 2, wherein the patient has been treated with an ICI therapy and has developed resistance to the ICI therapy.

Embodiment 5. The method of any one of embodiments 1-4, wherein the at least one AhR ligand comprises kynurenine, tryptophan, phenylpyruvate, indolelactate, or combinations thereof.

Embodiment 6. The method of embodiment 5, wherein the at least one AhR ligand comprises kynurenine, tryptophan, phenylpyruvate, and indolelactate.

Embodiment 7. The method of any one of embodiments 1-6, wherein the amount of the at least one AhR ligand in the sample is measured by liquid chromatography mass spectrometry or gas-phase chromatography mass spectrometry.

Embodiment 8. The method of any one of embodiments 1-7, wherein the threshold score is determined by measuring the amount of the corresponding at least one AhR ligand in a control serum, plasma, or fecal sample obtained from a patient who is responding to an ICI therapy.

Embodiment 9. The method of any one of embodiments 1-8, wherein the AhR antagonist is a compound of Formula I.

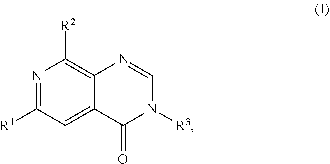

or a pharmaceutically acceptable salt thereof,

and wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines, and optionally substituted heterocycloalkyls, and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

Embodiment 10. The method of any one of embodiments 1-9, wherein the AhR antagonist is a compound of Formula Ia:

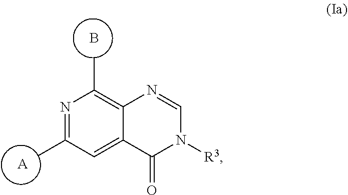

or a pharmaceutically acceptable salt thereof,

and wherein:
ring A is chosen from optionally substituted aryls, optionally substituted heteroaryls, optionally substituted cycloalkyls, and optionally substituted heterocycloalkyls,
ring B is chosen from optionally substituted aryls, optionally substituted heteroaryls, optionally substituted cycloalkyls, and optionally substituted heterocycloalkyls, and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

Embodiment 11. The method of any one of embodiments 1-9, wherein the AhR antagonist is any one listed in Table 2, or a pharmaceutically acceptable salt thereof.

Embodiment 12. An in vitro method of determining whether a patient would benefit from being treated with an aryl hydrocarbon receptor (AhR) antagonist, comprising: (a) measuring or having measured an amount of at least one AhR ligand from a serum, plasma, or fecal sample obtained from the patient, (b) comparing or having compared the amount of the at least one AhR ligand to a threshold score, and (c) determining that the patient would benefit from being treated with an AhR antagonist if the amount of the at least one AhR ligand is above the threshold score.

Embodiment 13. The method of embodiment 12, wherein the patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

Embodiment 14. The method of embodiment 12 or embodiment 13, wherein the patient has been treated with an immune-checkpoint inhibitor (ICI) therapy but is not responding to the ICI therapy.

Embodiment 15. The method of embodiment 12 or embodiment 13, wherein the patient has been treated with an ICI therapy and has developed resistance to the ICI therapy.

Embodiment 16. The method of any one of embodiments 12-15, wherein the at least one AhR ligand comprises kynurenine, tryptophan, phenylpyruvate, indolelactate, or combinations thereof.

Embodiment 17. The method of embodiment 16, wherein the at least one AhR ligand comprises kynurenine, tryptophan, phenylpyruvate, and indolelactate.

Embodiment 18. The method of any one of embodiments 12-17, wherein the amount of the at least one AhR ligand in the sample is measured by liquid chromatography mass spectrometry or gas-phase chromatography mass spectrometry.

Embodiment 19. The method of any one of embodiments 12-18, wherein the threshold score is determined by measuring the amount of the corresponding at least one AhR ligand in a control serum, plasma, or fecal sample obtained from a patient who is responding to an ICI therapy.

Embodiment 20. The method of any one of embodiments 11-19, wherein the AhR antagonist is a compound of Formula I:

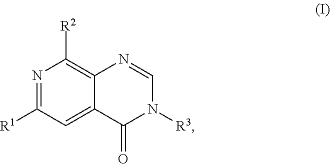

or a pharmaceutically acceptable salt thereof,

and wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines and optionally substituted heterocycloalkyls, and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

Embodiment 21. The method of any one of embodiments 12-20, wherein the AhR antagonist is a compound of Formula Ia:

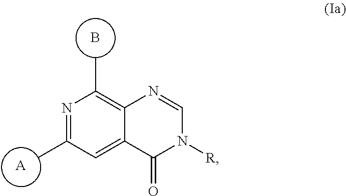

or a pharmaceutically acceptable salt thereof,

and wherein:
ring A is chosen from optionally substituted aryls, optionally substituted heteroaryls, optionally substituted cycloalkyls, and optionally substituted heterocycloalkyls,
ring B is chosen from optionally substituted aryls, optionally substituted heteroaryls, optionally substituted cycloalkyls, and optionally substituted heterocycloalkyls, and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

Embodiment 22. The method of any one of embodiments 12-20, wherein the AhR antagonist is any one listed in Table 2, or a pharmaceutically acceptable salt thereof.

Embodiment 23. A method of treating cancer in a patient in need thereof with a combination therapy comprising an immune checkpoint inhibitor (ICI) and an aryl hydrocarbon receptor (AhR) antagonist, comprising: (a) measuring or having measured an amount of at least one AhR ligand from a serum, plasma, or fecal sample obtained from the patient, (b) comparing or having compared the amount of the at least one AhR ligand to a threshold score, (c) determining or having determined that the patient would benefit from the combination therapy if the amount of the at least one AhR ligand is above the threshold score, and (d) treating the patient determined to benefit in (c) with the combination therapy.

Embodiment 24. The method of embodiment 23, wherein the patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

Embodiment 25. The method of embodiment 23 or embodiment 24, wherein the patient has been treated with an ICI therapy but is not responding to the ICI therapy.

Embodiment 26. The method of embodiment 23 or embodiment 24, wherein the patient has been treated with an ICI therapy and has developed resistance to the ICI therapy.

Embodiment 27. The method of any one of embodiments 23-26, wherein the at least one AhR ligand comprises kynurenine, tryptophan, phenylpyruvate, indolelactate, or combinations thereof.

Embodiment 28. The method of embodiment 27, wherein the at least one AhR ligand comprises kynurenine, tryptophan, phenylpyruvate, and indolelactate.

Embodiment 29. The method of any one of embodiments 23-28, wherein the amount of the at least one AhR ligand in the sample is measured by liquid chromatography mass spectrometry or gas-phase chromatography mass spectrometry.

Embodiment 30. The method of any one of embodiments 23-29, wherein the ICI therapy comprises an anti-PD-1 antibody, an anti-PD-L1 antibody, an anti-CTLA-4 antibody, an anti-LAG-3 antibody, an anti-TIM-3 antibody, an anti-TIGIT antibody, an anti-VISTA antibody, or combinations thereof.

Embodiment 31. The method of any one of embodiments 23-30, wherein the threshold score is determined by measuring the amount of the corresponding at least one AhR ligand in a control serum, plasma, or fecal sample obtained from a patient who is responding to an ICI therapy.

Embodiment 32. The method of any one of embodiments 23-31, wherein the AhR antagonist is a compound of Formula I.

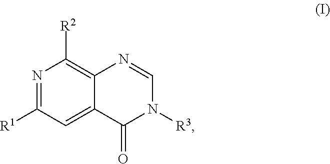

or a pharmaceutically acceptable salt thereof,

and wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines, and optionally substituted heterocycloalkyls, and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

Embodiment 33. The method of any one of embodiments 23-32, wherein the AhR antagonist is a compound of Formula Ia:

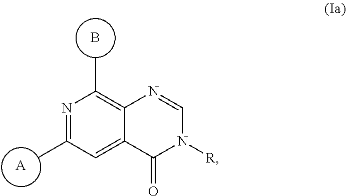

or a pharmaceutically acceptable salt thereof,

and wherein:
ring A is chosen from optionally substituted aryls, optionally substituted heteroaryls, optionally substituted cycloalkyls, and optionally substituted heterocycloalkyls,
ring B is chosen from optionally substituted aryls, optionally substituted heteroaryls, optionally substituted cycloalkyls, and optionally substituted heterocycloalkyls, and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

Embodiment 34. The method of any one of embodiments 23-33, wherein the AhR antagonist is any one listed in Table 2, or a pharmaceutically acceptable salt thereof.

Embodiment 35. An in vitro method of predicting a patient's cancer responsiveness to an immune checkpoint inhibitor (ICI) therapy, comprising: (a) measuring or having measured an amount of at least one aryl hydrocarbon receptor (AhR) ligand from a serum, plasma, or fecal sample obtained from the patient, (b) comparing or having compared the amount of the at least one AhR ligand to a threshold score, and (c) determining that the patient's cancer is likely to be responsive to an ICI therapy if the amount of the at least one AhR ligand is equal to or below the threshold score.

Embodiment 36. The method of embodiment 35, wherein the patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

Embodiment 37. The method of embodiment 35 or embodiment 36, wherein the at least one AhR ligand comprises kynurenine, tryptophan, phenylpyruvate, indolelactate, or combinations thereof.

Embodiment 38. The method of embodiment 37, wherein the at least one AhR ligand comprises kynurenine, tryptophan, phenylpyruvate, and indolelactate.

Embodiment 39. The method of any one of embodiments 35-38, wherein the amount of the at least one AhR ligand in the sample is measured by liquid chromatography mass spectrometry or gas-phase chromatography mass spectrometry.

Embodiment 40. The method of any one of embodiments 35-39, wherein the ICI therapy comprises an anti-PD-1 antibody, an anti-PD-L1 antibody, an anti-CTLA-4 antibody, an anti-LAG-3 antibody, an anti-TIM-3 antibody, an anti-TIGIT antibody, an anti-VISTA antibody, or combinations thereof.

Embodiment 41. The method of any one of embodiments 35-40, wherein the threshold score is determined by measuring the amount of the corresponding at least one AhR ligand in a control serum, plasma, or fecal sample obtained from a patient who is responding to an ICI therapy.

Embodiment 42. The method of any one of embodiments 35-41, further comprising determining that the patient's cancer is likely to be responsive to an ICI therapy in combination with an AhR antagonist if the amount of the at least one AhR ligand is above the threshold score.

Embodiment 43. The method of embodiment 42, wherein the AhR antagonist is a compound of Formula I:

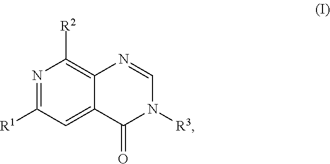

or a pharmaceutically acceptable salt thereof,

and wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines and optionally substituted heterocycloalkyls, and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

Embodiment 44. The method of embodiment 42 or embodiment 43, wherein the AhR antagonist is a compound of Formula Ia:

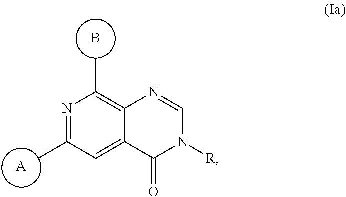

or a pharmaceutically acceptable salt thereof,

and wherein:
ring A is chosen from optionally substituted aryls, optionally substituted heteroaryls, optionally substituted cycloalkyls, and optionally substituted heterocycloalkyls,
ring B is chosen from optionally substituted aryls, optionally substituted heteroaryls, optionally substituted cycloalkyls, and optionally substituted heterocycloalkyls, and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

Embodiment 45. The method of any one of embodiments 42-44, wherein the AhR antagonist is any one listed in Table 2, or a pharmaceutically acceptable salt thereof.

Embodiment 46. A method of treating cancer in a patient in need thereof, comprising treating the patient with an effective amount of an aryl hydrocarbon receptor (AhR) antagonist, wherein a serum, plasma, or fecal sample obtained from the patient has an amount of at least one AhR ligand above a threshold score.

Embodiment 47. The method of embodiment 46, wherein the patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

Embodiment 48. The method of embodiment 46 or embodiment 47, wherein the patient has been treated with an immune-checkpoint inhibitor (ICI) therapy but is not responding to the ICI therapy.

Embodiment 49. The method of embodiment 46 or embodiment 47, wherein the patient has been treated with an ICI therapy and has developed resistance to the ICI therapy.

Embodiment 50. The method of any one of embodiments 46-49, wherein the at least one AhR ligand comprises kynurenine, tryptophan, phenylpyruvate, indolelactate, or combinations thereof.

Embodiment 51. The method of embodiment 50, wherein the at least one AhR ligand comprises kynurenine, tryptophan, phenylpyruvate, and indolelactate.

Embodiment 52. The method of any one of embodiments 46-51, wherein the amount of the at least one AhR ligand in the sample is measured by liquid chromatography mass spectrometry or gas-phase chromatography mass spectrometry.

Embodiment 53. The method of any one of embodiments 46-52, wherein the threshold score is determined by measuring the amount of the corresponding at least one AhR ligand in a control serum, plasma, or fecal sample obtained from a patient who is responding to an ICI therapy.

Embodiment 54. The method of any one of embodiments 46-53, wherein the AhR antagonist is a compound of Formula I:

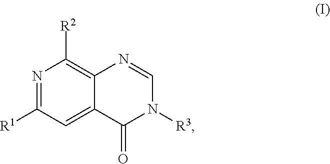

or a pharmaceutically acceptable salt thereof,

and wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines, and optionally substituted heterocycloalkyls, and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

Embodiment 55. The method of any one of embodiments 46-53, wherein the AhR antagonist is a compound of Formula Ia:

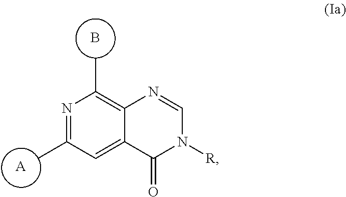

or a pharmaceutically acceptable salt thereof,

and wherein:
ring A is chosen from optionally substituted aryls, optionally substituted heteroaryls, optionally substituted cycloalkyls, and optionally substituted heterocycloalkyls,
ring B is chosen from optionally substituted aryls, optionally substituted heteroaryls, optionally substituted cycloalkyls, and optionally substituted heterocycloalkyls, and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

Embodiment 56. The method of any one of embodiments 46-55, wherein the AhR antagonist is any one listed in Table 2, or a pharmaceutically acceptable salt thereof.

Embodiment 57. A method of treating cancer in a patient in need thereof, comprising treating the patient with a combination therapy comprising an immune checkpoint inhibitor (ICI) and an aryl hydrocarbon receptor (AhR) antagonist, wherein a serum, plasma, or fecal sample obtained from the patient has an amount of at least one AhR ligand above a threshold score.

Embodiment 58. The method of embodiment 57, wherein the patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

Embodiment 59. The method of embodiment 57 or embodiment 58, wherein the patient has been treated with an immune-checkpoint inhibitor (ICI) therapy but is not responding to the ICI therapy.

Embodiment 60. The method of embodiment 57 or embodiment 58, wherein the patient has been treated with an ICI therapy and has developed resistance to the ICI therapy.

Embodiment 61. The method of any one of embodiments 57-60, wherein the at least one AhR ligand comprises kynurenine, tryptophan, phenylpyruvate, indolelactate, or combinations thereof.

Embodiment 62. The method of embodiment 61, wherein the at least one AhR ligand comprises kynurenine, tryptophan, phenylpyruvate, and indolelactate.

Embodiment 63. The method of any one of embodiments 57-62, wherein the amount of the at least one AhR ligand in the sample is measured by liquid chromatography mass spectrometry or gas-phase chromatography mass spectrometry.

Embodiment 64. The method of any one of embodiments 57-63, wherein the ICI therapy comprises an anti-PD-1 antibody, an anti-PD-L1 antibody, an anti-CTLA-4 antibody, an anti-LAG-3 antibody, an anti-TIM-3 antibody, an anti-TIGIT antibody, an anti-VISTA antibody, or combinations thereof.

Embodiment 65. The method of any one of embodiments 57-64, wherein the threshold score is determined by measuring the amount of the corresponding at least one AhR ligand in a control serum, plasma, or fecal sample obtained from a patient who is responding to an ICI therapy.

Embodiment 66. The method of any one of embodiments 57-65, wherein the AhR antagonist is a compound of Formula I:

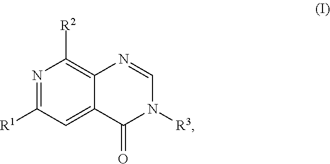

or a pharmaceutically acceptable salt thereof,

and wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines, and optionally substituted heterocycloalkyls, and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

Embodiment 67. The method of any one of embodiments 57-66, wherein the AhR antagonist is a compound of Formula Ia:

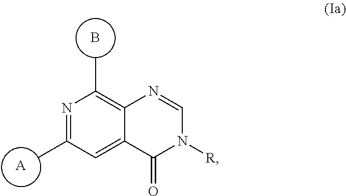

or a pharmaceutically acceptable salt thereof,

and wherein:
ring A is chosen from optionally substituted aryls, optionally substituted heteroaryls, optionally substituted cycloalkyls, and optionally substituted heterocycloalkyls,
ring B is chosen from optionally substituted aryls, optionally substituted heteroaryls, optionally substituted cycloalkyls, and optionally substituted heterocycloalkyls, and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

Embodiment 68. The method of any one of embodiments 57-66, wherein the AhR antagonist is any one listed in Table 2, or a pharmaceutically acceptable salt thereof.

Embodiment 69. An aryl hydrocarbon receptor (AhR) antagonist for use in a method of treating cancer in a patient in need thereof, wherein a serum, plasma, or fecal sample obtained from the patient has an amount of at least one AhR ligand above a threshold score.

Embodiment 70. The AhR antagonist for use of embodiment 69, wherein the patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

Embodiment 71. The AhR antagonist for use of embodiment 69 or embodiment 70, wherein the patient has been treated with an immune-checkpoint inhibitor (ICI) therapy but is not responding to the ICI therapy.

Embodiment 72. The AhR antagonist for use of embodiment 69 or embodiment 70, wherein the patient has been treated with an ICI therapy and has developed resistance to the ICI therapy.

Embodiment 73. The AhR antagonist for use of any one of embodiments 69-72, wherein the at least one AhR ligand comprises kynurenine, tryptophan, phenylpyruvate, indolelactate, or combinations thereof.

Embodiment 74. The AhR antagonist for use of embodiment 73, wherein the at least one AhR ligand comprises kynurenine, tryptophan, phenylpyruvate, and indolelactate.

Embodiment 75. The AhR antagonist for use of any one of embodiments 69-74, wherein the amount of the at least one AhR ligand in the sample is measured by liquid chromatography mass spectrometry or gas-phase chromatography mass spectrometry.

Embodiment 76. The AhR antagonist for use of any one of embodiments 69-75, wherein the threshold score is determined by measuring the amount of the corresponding at least one AhR ligand in a control serum, plasma, or fecal sample obtained from a patient who is responding to an ICI therapy.

Embodiment 77. The AhR antagonist for use of any one of embodiments 69-76, wherein the AhR antagonist is a compound of Formula I:

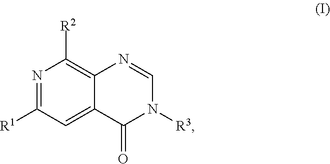

or a pharmaceutically acceptable salt thereof,

and wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines, and optionally substituted heterocycloalkyls, and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

Embodiment 78. The AhR antagonist for use of any one of embodiments 69-77, wherein the AhR antagonist is a compound of Formula Ia:

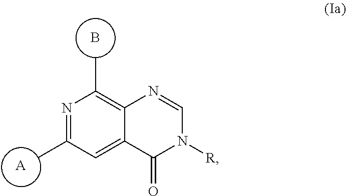

or a pharmaceutically acceptable salt thereof,

and wherein:
ring A is chosen from optionally substituted aryls, optionally substituted heteroaryls, optionally substituted cycloalkyls, and optionally substituted heterocycloalkyls,
ring B is chosen from optionally substituted aryls, optionally substituted heteroaryls, optionally substituted cycloalkyls, and optionally substituted heterocycloalkyls, and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

Embodiment 79. The AhR antagonist for use of any one of embodiments 69-77, wherein the AhR antagonist is any one listed in Table 2, or a pharmaceutically acceptable salt thereof.

Embodiment 80. A combination therapy for use in a method of treating cancer in a patient in need thereof, wherein the combination therapy comprises an immune checkpoint inhibitor (ICI) and an aryl hydrocarbon receptor (AhR) antagonist, and further wherein a serum, plasma, or fecal sample obtained from the patient has an amount of at least one AhR ligand above a threshold score.

Embodiment 81. The combination therapy for use of embodiment 80, wherein the patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

Embodiment 82. The combination therapy for use of embodiment 80 or embodiment 81, wherein the patient has been treated with an immune-checkpoint inhibitor (ICI) therapy but is not responding to the ICI therapy.

Embodiment 83. The combination therapy for use of embodiment 80 or embodiment 81, wherein the patient has been treated with an ICI therapy and has developed resistance to the ICI therapy.

Embodiment 84. The combination therapy for use of any one of embodiments 80-83, wherein the at least one AhR ligand comprises kynurenine, tryptophan, phenylpyruvate, indolelactate, or combinations thereof.

Embodiment 85. The combination therapy for use of embodiment 84, wherein the at least one AhR ligand comprises kynurenine, tryptophan, phenylpyruvate, and indolelactate.

Embodiment 86. The combination therapy for use of any one of embodiments 80-85, wherein the amount of the at least one AhR ligand in the sample is measured by liquid chromatography mass spectrometry or gas-phase chromatography mass spectrometry.

Embodiment 87. The combination therapy for use of any one of embodiments 80-86, wherein the ICI therapy comprises an anti-PD-1 antibody, an anti-PD-L1 antibody, an anti-CTLA-4 antibody, an anti-LAG-3 antibody, an anti-TIM-3 antibody, an anti-TIGIT antibody, an anti-VISTA antibody, or combinations thereof.

Embodiment 88. The combination therapy for use of any one of embodiments 80-87, wherein the threshold score is determined by measuring the amount of the corresponding at least one AhR ligand in a control serum, plasma, or fecal sample obtained from a patient who is responding to an ICI therapy.

Embodiment 89. The combination therapy for use of any one of embodiments 80-88, wherein the AhR antagonist is a compound of Formula I:

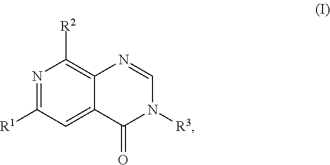

or a pharmaceutically acceptable salt thereof,

and wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines, and optionally substituted heterocycloalkyls, and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

Embodiment 90. The combination therapy for use of any one of embodiments 80-89, wherein the AhR antagonist is a compound of Formula Ia:

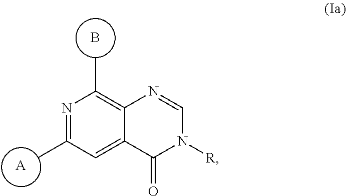

or a pharmaceutically acceptable salt thereof,

and wherein:
ring A is chosen from optionally substituted aryls, optionally substituted heteroaryls, optionally substituted cycloalkyls, and optionally substituted heterocycloalkyls,
ring B is chosen from optionally substituted aryls, optionally substituted heteroaryls, optionally substituted cycloalkyls, and optionally substituted heterocycloalkyls, and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

Embodiment 91. The combination therapy for use of any one of embodiments 80-89, wherein the AhR antagonist is any one listed in Table 2, or a pharmaceutically acceptable salt thereof.

Embodiment 92. Use of an aryl hydrocarbon receptor (AhR) antagonist in the manufacture of a medicament for treating cancer in a patient in need thereof, wherein a serum, plasma, or fecal sample obtained from the patient has an amount of at least one AhR ligand above a threshold score.

Embodiment 93. The use of embodiment 92, wherein the patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

Embodiment 94. The use of embodiment 92 or embodiment 93, wherein the patient has been treated with an immune-checkpoint inhibitor (ICI) therapy but is not responding to the ICI therapy.

Embodiment 95. The use of embodiment 92 or embodiment 93, wherein the patient has been treated with an ICI therapy and has developed resistance to the ICI therapy.

Embodiment 96. The use of any one of embodiments 92-95, wherein the at least one AhR ligand comprises kynurenine, tryptophan, phenylpyruvate, indolelactate, or combinations thereof.

Embodiment 97. The use of embodiment 96, wherein the at least one AhR ligand comprises kynurenine, tryptophan, phenylpyruvate, and indolelactate.

Embodiment 98. The use of any one of embodiments 92-97, wherein the amount of the at least one AhR ligand in the sample is measured by liquid chromatography mass spectrometry or gas-phase chromatography mass spectrometry.

Embodiment 99. The use of any one of embodiments 92-98, wherein the threshold score is determined by measuring the amount of the corresponding at least one AhR ligand in a control serum, plasma, or fecal sample obtained from a patient who is responding to an ICI therapy.

Embodiment 100. The use of any one of embodiments 92-99, wherein the AhR antagonist is a compound of Formula I:

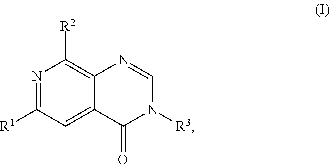

or a pharmaceutically acceptable salt thereof,

and wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines, and optionally substituted heterocycloalkyls, and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

Embodiment 101. The use of any one of embodiments 92-100, wherein the AhR antagonist is a compound of Formula Ia:

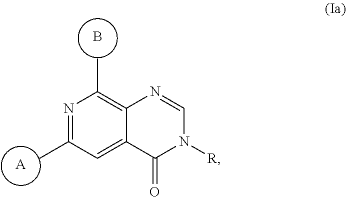

or a pharmaceutically acceptable salt thereof,

and wherein:
ring A is chosen from optionally substituted aryls, optionally substituted heteroaryls, optionally substituted cycloalkyls, and optionally substituted heterocycloalkyls,
ring B is chosen from optionally substituted aryls, optionally substituted heteroaryls, optionally substituted cycloalkyls, and optionally substituted heterocycloalkyls, and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

Embodiment 102. The use of any one of embodiments 92-100, wherein the AhR antagonist is any one listed in Table 2, or a pharmaceutically acceptable salt thereof.

Embodiment 103. Use of an immune checkpoint inhibitor (ICI) and an aryl hydrocarbon receptor (AhR) antagonist in the manufacture of a medicament for treating cancer in a patient in need thereof, wherein a serum, plasma, or fecal sample obtained from the patient has an amount of at least one AhR ligand above a threshold score.

Embodiment 104. The use of embodiment 103, wherein the patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

Embodiment 105. The use of embodiment 103 or embodiment 104, wherein the patient has been treated with an immune-checkpoint inhibitor (ICI) therapy but is not responding to the ICI therapy.

Embodiment 106. The use of embodiment 103 or embodiment 104, wherein the patient has been treated with an ICI therapy and has developed resistance to the ICI therapy.

Embodiment 107. The use of any one of embodiments 103-106, wherein the at least one AhR ligand comprises kynurenine, tryptophan, phenylpyruvate, indolelactate, or combinations thereof.

Embodiment 108. The use of embodiment 107, wherein the at least one AhR ligand comprises kynurenine, tryptophan, phenylpyruvate, and indolelactate.

Embodiment 109. The use of any one of embodiments 103-108, wherein the amount of the at least one AhR ligand in the sample is measured by liquid chromatography mass spectrometry or gas-phase chromatography mass spectrometry.

Embodiment 110. The use of any one of embodiments 103-109, wherein the ICI therapy comprises an anti-PD-1 antibody, an anti-PD-L1 antibody, an anti-CTLA-4 antibody, an anti-LAG-3 antibody, an anti-TIM-3 antibody, an anti-TIGIT antibody, an anti-VISTA antibody, or combinations thereof.

Embodiment 111. The use of any one of embodiments 103-110, wherein the threshold score is determined by measuring the amount of the corresponding at least one AhR ligand in a control serum, plasma, or fecal sample obtained from a patient who is responding to an ICI therapy.

Embodiment 112. The use of any one of embodiments 103-111, wherein the AhR antagonist is a compound of Formula I.

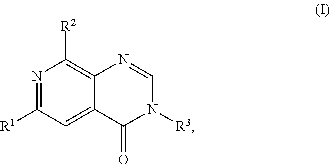

or a pharmaceutically acceptable salt thereof,

and wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines, and optionally substituted heterocycloalkyls, and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

Embodiment 113. The use of any one of embodiments 103-112, wherein the AhR antagonist is a compound of Formula Ia:

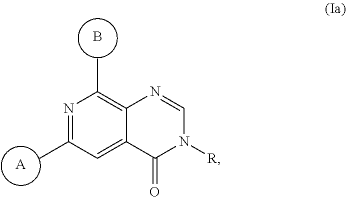

or a pharmaceutically acceptable salt thereof,
and wherein:
ring A is chosen from optionally substituted aryls, optionally substituted heteroaryls, optionally substituted cycloalkyls, and optionally substituted heterocycloalkyls,
ring B is chosen from optionally substituted aryls, optionally substituted heteroaryls, optionally substituted cycloalkyls, and optionally substituted heterocycloalkyls, and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

Embodiment 114. The use of any one of embodiments 103-112, wherein the AhR antagonist is any one listed in Table 2, or a pharmaceutically acceptable salt thereof.

### DESCRIPTION OF DRAWINGS

FIGS. 1A-1B show the levels of kynurenine, tryptophan, phenylpyruvate, and indolelactate (collectively, associated ligands) from fecal samples (FIG. 1A) and plasma samples (FIG. 1B) obtained from 73 patients of Example 1 that eventually went on to be characterized as either responders or non-responders.

FIG. 2 shows elevated levels of the associated ligands from fecal samples obtained from 19 patients receiving ICI therapy only (no concomitant therapy) that eventually went on to be characterized as either responders or non-responders.

FIG. 3 shows a principal component analysis for dimensionality reduction of signals from the full panel of 29 AhR ligands compared to the associated ligands. Left panel: 73 patients—29 AhR ligands; middle panel: 73 patients—associated ligands; right panel: 19 patients receiving ICI therapy only—associated ligands.

FIGS. 4A-4B show the tumor growth curves (FIG. 4A) and tumor weight (FIG. 4B) measured after administering an isotope control antibody, an anti-PD-L1 antibody, and Compound No. 30 of Table 2 in combination with anti-PD-L1 antibody (BioXcell anti-mouse PD-L1, B7-H1 clone 10F.9G2), using a colon cancer mouse model resistant to anti-PD-L1 therapy.

FIG. 5 shows the levels of AhR ligands described in Table 1 in patients receiving ICI therapy only (no concomitant therapy) that eventually went on to be characterized as either responders or non-responders.

The present disclosure is based in part on the discovery that certain AhR ligands, such as kynurenine, tryptophan, phenylpyruvate, and indolelactate, are useful biomarkers predictive of a patient's cancer responsiveness to an ICI therapy. In some embodiments, the present disclosure provides methods of predicting a patient's cancer responsiveness to an ICI therapy based upon a determination of the amount of at least one biomarker relative to a threshold score. In some embodiments, the biomarkers described herein may enable identification and/or stratification of cancer patients that would benefit from being treated with an AhR antagonist. In some embodiments, the biomarkers described herein may be used to inform and provide effective therapeutic methods for cancer patients subject to one or more ICI therapies.

It should be understood that references herein to methods of treatment (e.g., methods of treating cancer) using an aryl hydrocarbon receptor (AhR) antagonist or a combination therapy should also be interpreted as references to:
an AhR antagonist or a combination therapy for use in methods of treating, e.g., cancer; and/or
the use of an AhR antagonist or a combination therapy in the manufacture of a medicament for treating, e.g., cancer.

The following detailed description and examples illustrate certain embodiments of the present disclosure. Those of skill in the art will recognize that there are numerous variations and modifications of the disclosure that are encompassed by its scope. Accordingly, the description of certain embodiments should not be deemed to limit the scope of the present disclosure.

### Definitions

In order that the disclosure may be more readily understood, certain terms are defined throughout the detailed description. Unless defined otherwise herein, all scientific and technical terms used in connection with the present disclosure have the same meaning as commonly understood by those of ordinary skill in the art.

As used herein, the singular forms of a word also include the plural form, unless the context clearly dictates otherwise; as examples, the terms “a,” “an,” and “the” are understood to be singular or plural. The term “or” shall mean “and/or” unless the specific context indicates otherwise.

As used herein, the term “biomarker” refers to a biological compound that is present in a biological sample and may be isolated from, or measured in, the biological sample. Such biomarkers include, without limitation, nucleic acids, proteins, carbohydrates, lipids, organic or inorganic chemicals, natural polymers, and metabolites. In some embodiments, the biomarker includes one or more AhR ligands, such as kynurenine, tryptophan, phenylpyruvate, and indolelactate, as shown in Example 1. In some embodiments, the biomarker according to the disclosure can be predictive of a cancer patient's responsiveness to ICI therapy.

As used herein, the term “measuring” or “detecting” refers to assessing the presence, absence, or amount of a biomarker within a biological sample disclosed herein, including the derivation of quantitative levels of such a biomarker. In some embodiments, the amount of the at least one biomarker in a biological sample is measured or detected using methods known in the art, such as liquid chromatography mass spectrometry and gas-phase chromatography mass spectrometry.

As used herein, the term “threshold score” refers to a reference biomarker amount that is suitable to provide a comparison to the amount of that biomarker in the test sample. In some embodiments, the threshold score is the amount of the biomarker in a serum, plasma, or fecal sample from a cancer patient who is responding to an ICI therapy. In some embodiments, the threshold score can be generated from a population or cohort of two or more cancer patients who are responding to ICI therapies. The population or cohort, for example, may comprise at least 2, 3, 4, 5, 10, 15, 18, 20, 30, 40, 50, 75, 100 or more cancer patients. In such embodiments, the threshold score for each biomarker may be the average amount of that biomarker in serum, plasma, or fecal samples from a population or cohort of cancer patients who are responding to ICI therapies.

The terms “biological sample” and “sample” are used interchangeably to refer to a specimen obtained from a patient including body fluids, body tissue (e.g., tumor tissue), cells, or other sources. In some embodiments, the biological sample includes feces obtained from a cancer patient subject to an ICI therapy. In some embodiments, the biological sample includes plasma obtained from a cancer patient subject to an ICI therapy. In some embodiments, the biological sample includes feces obtained from a cancer patient who has not been administered an ICI therapy. In some embodiments, the biological sample includes plasma obtained from a cancer patient who has not been administered an ICI therapy. Methods for obtaining such biological samples from cancer patients are known in the art.

As used herein, the term “AhR ligand” refers to a compound which binds to and activates the aryl hydrocarbon receptor (i.e., AhR) expressed in many cells of the immune system and, upon binding, causes the translocation of the AhR to the nucleus where it interacts with Dioxin Response Elements (DREs) of AhR-responsive genes to regulate their transcription. AhR ligands are known in the art and include classic xenobiotics such as 2,3,7,8-Tetrachlorodibenzo-p-dioxin (TCDD) and 3-methylcholanthrene. Additional examples of AhR ligands include kynurenine, tryptophan, phenylpyruvate, indolelactate, beta-naphthoflavone (BNF), 6-formylindolo(3,2-b) carbazole (FICZ), and 2-(1H-indole-3-ylcarbonyl)-4-thiazolecarboxylic methyl ester (ITE). Furthermore, Table 1 lists 29 AhR ligands investigated in Example 1, including kynurenine, tryptophan, phenylpyruvate, and indolelactate.

As used herein, the term “AhR antagonist” refers to any agent capable of inhibiting or reducing the biological activity of the AhR to which it binds. In some embodiments, the AhR antagonist reduces the activity of the AhR by at least about 5%, 10%, 20%, 30%, 40%, 50%, 60%, 70%, 80%, 90%, 95%, or 99%, relative to the activity of the AhR in the absence of the AhR antagonist. Certain AhR antagonists such as α-naphthoflavone and resveratrol are known in the art. Additional AhR antagonists are described in the disclosure. See, e.g., Table 2.

The terms “comprise,” “contain,” “have,” and “include” are open-ended linking verbs. Any forms or tenses of one or more of these verbs, such as “comprises,” “comprising,” “contains,” “containing,” “has,” “having,” “includes,” and “including,” are also open-ended. For example, any method that “comprises,” “contains,” “has” or “includes” one or more steps is not limited to possessing only those one or more steps and can also cover other unlisted steps. The use of any and all examples, or exemplary language (e.g., “such as”) provided with respect to certain embodiments herein is intended merely to better illuminate the present disclosure and does not pose a limitation on the scope of the present disclosure otherwise claimed.

The terms “patient” and “subject” are used interchangeably herein to refer to a human or non-human animal (e.g., a mammal). As used herein, the term “cancer patient” refers to a patient who has been diagnosed by a qualified professional (e.g., a doctor or a nurse practitioner) as having a cancer or tumor.

The terms “cancer” or “tumor” are used interchangeably herein to refer to the presence of cells possessing characteristics typical of cancer-causing cells, such as uncontrolled proliferation, immortality, metastatic potential, rapid growth and proliferation rate, and certain characteristic morphological features. In some embodiments, such cells exhibit such characteristics in part or in full due to the expression and activity of immune checkpoint inhibitors, such as PD-1, PD-L1, and/or CTLA-4. Cancers include, but are not limited to, non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, and liver cancer.

As used herein, the term “immune checkpoint” refers to a group of molecules on the cell surface of CD4+ and/or CD8+ T cells that down-modulate or inhibit immune responses to maintain self-tolerance, prevent autoimmunity, and control the duration and extent of immune responses in order to minimize collateral tissue damage. Immune checkpoint proteins are known in the art and include, without limitation, CTLA-4, PD-1, PD-L1, VISTA, B7-H2, B7-H3, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, TLT-2, ILT-4, TIGIT, HHLA2, butyrophilins, IDO, CD39, CD73, and A2aR.

As used here, the term “immune checkpoint inhibitor” (ICI) refers to any therapeutic agent, including any small molecule chemical compound, nucleic acid molecule, or polypeptide (e.g., antibody), or any fragments thereof, that inhibits one or more immune checkpoint proteins. Inhibition of one or more immune checkpoint proteins can block or otherwise neutralize inhibitory signaling to thereby upregulate an immune response in order to, for example, more efficaciously treat cancer. Exemplary immune checkpoint inhibitors include antibodies against one or more immune checkpoint proteins that directly block the interaction between the proteins and their natural receptors, a non-activating form of one or more immune checkpoint proteins, small molecules or peptides that block the interaction between one or more immune checkpoint proteins and their natural receptors, nucleic acid molecules that block immune checkpoint protein nucleic acid transcription or translation, and the like.

As used herein, the term “treating” or “treatment” refers to alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof. In some embodiments, the condition is cancer. With respect to treating cancer, the term “treating” or “treatment” may refer to providing an ICI therapy optionally in combination with an AhR antagonist to a cancer patient such that at least one or more symptoms of the cancer is ameliorated.

As used herein, the term “effective amount” refers to the amount of a therapeutic agent or a pharmaceutically acceptable salt thereof sufficient to reduce at least one or more symptoms of a condition disclosed herein, or to provide a desired effect. With respect to treating cancer, an “effective amount” may vary according to factors such as the cancer stage and/or age, sex, and weight of the cancer patient.

As used herein, the term “administering” refers to the placement of a therapeutic agent (e.g., an AhR antagonist or an ICI) into a mammalian tissue or a subject by a method or route that results in at least partial localization of the therapeutic agent at a desired site or tissue location. In some embodiments, a therapeutic agent may be administered to a subject by using routes known in art, such as intramuscular injection, subcutaneous injection, and intravenous injection or infusion.

As used herein, the term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 25% or more, for example, 30%, 40%, 50%, 60%, 70%, 80%, or more. The reduction in response can be measured by comparing with the same cancer sample or mammal before the resistance is acquired, or by comparing with a different cancer sample or a mammal that is known to have no resistance to the therapeutic treatment. The determination of resistance to a therapeutic treatment is routine in the art and within the skill of an ordinarily skilled clinician.

The term “respond,” “response,” or “responsiveness” refers to an anti-cancer response, e.g., in the sense of reduction of tumor size or inhibiting tumor growth. The term can also refer to an improved prognosis, for example, as reflected by an increased time to recurrence, or an increased overall survival, which is the period from treatment to death from any cause. To respond or to have a response means there is a beneficial endpoint attained when exposed to a stimulus. Alternatively, to respond or to have a response means that a negative or detrimental symptom is minimized, mitigated or attenuated on exposure to a stimulus. The determination of response or responsiveness to a therapeutic treatment is routine in the art and within the skill of an ordinarily skilled clinician. For example, whether a patient is responsive to an ICI therapy can be determined based on the adopted response evaluation criteria in solid tumors (RECIST) criteria. See Example 1.

As used herein, the term “combination therapy” refers to delivering two or more different treatments to a patient during the patient's affliction with a disease, disorder, or condition (e.g., cancer). For example, in some embodiments, the two treatments, such as an ICI therapy and an AhR antagonist, are delivered after the patient has been diagnosed with cancer, and before the cancer has been cured or eliminated. In some embodiments, the delivery of one treatment is still occurring when the delivery of the second treatment begins, so that there is an overlap between the two. In some embodiments, the first and second treatment are initiated at the same time. In other embodiments, the first and second treatment are initiated at different times. In some embodiments, one treatment ends before the second treatment begins. In some embodiments, an ICI therapy and an AhR antagonist are administered simultaneously. In some embodiments, an ICI therapy and an AhR antagonist are administered sequentially. In some embodiments, the two treatments are administered sufficiently close in time so as to provide the desired therapeutic effect.

### AhR Ligands as Biomarkers for ICI Responsiveness

According to the present disclosure, certain AhR ligands, such as kynurenine, tryptophan, phenylpyruvate, and indolelactate, are useful as biomarkers for predicting a patient's cancer responsiveness to ICI therapy.

The AhR is a ligand-activated transcription factor belonging to the basic helix-loop-helix/Per-Amt-Sim (bHLH/PAS) protein family that is located in the cytosol. Upon ligand binding, the AhR translocates to the nucleus where it heterodimerizes with AhR nuclear translocator (ARNT) upon which it interacts with dioxin response elements (DREs) of AhR-responsive genes to regulate their transcription. Representative target genes include the microsomal cytochrome P450-dependent monooxygenases including cytochrome P450 family-1 subfamily-A polypeptide-1 (CYP1A1), cytochrome P450 family-1 subfamily-A Polypeptide-2 (CYP1A2), cytochrome P450 family-1 subfamily-B polypeptide-1 (CYP1B1), and NAD(P)H-quinone oxidoreductase. AhR can also influence chromatin remodeling, acting on deacetylase and proofreading its gene regulatory activity to direct gene repression (Rothhammer et al., Nat Rev Immunol., 2019, 19(3):184-197). In addition, AhR interacts with transcription factors such as NF-κB, c-Maf, and a series of other gene regulators and influences the activities of these regulators (Hankinson et al., Arch Biochem Biophys., 2005, 433(2):379-386). A feedback loop for NF-κB activation has also been described.

AhR is expressed in many cells of the immune system, including dendritic cells (DCs), macrophages, T cells, and NK cells, and plays a role in immunoregulation (Nguyen et al., Front. Immunol., 2014, 5:551). There are multiple reports that AhR mediates the differentiation of CD4(+) T-cell polarization, specifically Th17 and Th22 differentiation, and exacerbates autoimmunity in mice (Funatake et al., J Immunol., 2005, 175(7):4184-4188; Quintana et al., Nature, 2008, 453(7191):65-71; Veldhoen et al., Nature, 2008, 453(7191):106-109; Xiong et al., Exp Cell Res., 2020, 112288). AhR also regulates the tone of the adaptive immune response, modulating T-cell differentiation toward a regulatory phenotype. Furthermore, Quintana and others reported a role for AhR in the regulation of Th17 biology and control of IL-22 production (Quintana et al., Nature, 2008, 453(7191):65-71; Quintana et al., Eur J Immunol., 2009, 39(3):655-657; Yeste et al., Nat Commun., 2014, 5:3753).

In the context of immunosuppression, AhR activation promotes regulatory T cell generation, inhibits Th1 and Th17 differentiation, directly and indirectly, and decreases the activation and maturation of dendritic cells (DCs) (Wang et al., Clin. Exp. Immunol., 2014, 177(2):521-30; Mezrich et al., J. Immunol., 2010, 185(6):3190-8; Wei et al., Lab. Invest., 2014, 94(5):528-35; Nguyen et al., PNAS, 2010, 107(46):19961-6). The classic exogenous AhR ligands, TCDD and 3-methylcholanthrene, are known to induce profound immunosuppression, promote carcinogenesis, and induce tumor growth (Gramatzki et al., Oncogene, 2009, 28(28):2593-605; Bui et al., Oncogene, 2009, 28(41):3642-51; Esser et al., Trends Immunol., 2009, 30:447-454).

AhR activation also modulates the innate immune response and constitutive AhR expression has been shown to negatively regulate the type-1 interferon response to viral infection (Yamada et al., Nat. Immunol., 2016, 17(6):687-94).

Mice with a constitutively active AhR spontaneously develop tumors (Andersson et al., PNAS, 2002, 99(15):9990-5). There is evidence that AhR activity is dysregulated within the tumor environment, affecting the immune cells and causing impaired immune surveillance. For example, AhR can affect tumorigenesis, directly perturbing cell proliferation, tissue invasion, angiogenesis, tumor-associated inflammation, and metastasis (Xiong et al., Exp Cell Res., 2020, 112288; Veldhoen et al., Nature, 2008, 453(7191):106-109; Dietrich et al., Carcinogenesis, 2010, 31(8):1319-1328; Opitz et al., Nature, 2011, 478(7368):197-203).

AhR signaling can be activated by one or more AhR ligands known in the art. For example, AhR can be activated by environmental toxins like TCDD and by natural ligands provided directly by the diet, or dietary substances further transformed by enzymatic activity of the gut microbiomes. These ligands, in turn, modulate the transcriptional activity of AhR in the host immune system, influencing the immune response towards the microbiome. Tryptophan metabolites, such as kynurenine and kynurenic acid, have been reported to activate AhR (DiNatale et al., Toxicol. Sci., 2010, 115(1):89-97; Mezrich et al., J. Immunol., 2010, 185(6):3190-8; Opitz et al., Nature, 2011, 478(7368):197-203). Other endogenous ligands are known to bind the AHR, although their physiological roles are currently unknown (Nguyen & Bradfield, Chem. Res. Toxicol., 2008, 21(1):102-116).

It has been suggested that the essential amino acid tryptophan is catabolized in tumor tissue by the rate-limiting enzyme indoleamine-2,3-dioxygenase 1 and 2 (IDO1/IDO2) expressed in tumor cells, myeloid or antigen-presenting cells and by tryptophan-2,3-dioxygenase 2 (TDO2) (Platten et al., Front Immunol., 2015, 5:673). The IDO1/2-mediated metabolic pathway creates an immune-permissive microenvironment in tumors and in tumor-draining lymph nodes by inducing T-cell anergy and apoptosis through depletion of tryptophan and accumulation of immunosuppressive tryptophan catabolites (Fallarino et al., Cell Death Differ., 2002, 9(10):1069-1077; Uyttenhove et al., Nat. Med., 2003, 9(10):1269-74; Liu et al., Blood, 2005, 115(17):3520-30; Muller et al., Nat. Med., 11(3):312-9; Metz, Cancer Res., 2007, 67(15):7082-7). Competitive inhibitors of IDO are currently being tested in clinical trials in patients with solid cancer, with the aim of enhancing the efficacy of conventional chemotherapy.

TDO2 is strongly expressed in cancer and can lead to the production of immunosuppressive kynurenine. In glioma, activation of the AhR by kynurenine, downstream of TDO-mediated tryptophan degradation, enhances tumor growth as a consequence of inhibiting anti-tumor immune responses as well as directly promoting tumor cell survival and motility (Opitz et al., Nature, 2011, 478(7368):197-203). AhR ligands generated by tumor cells therefore act in both an autocrine and paracrine fashion on tumor cells and lymphocytes, respectively, to promote tumor growth.

Constitutive activation of the IDO1/TDO2/AhR pathway and nuclear AhR protein accumulation is linked to non-response to immune checkpoint inhibitors and poor overall survival (Chen et al., Medicine (Baltimore), 2020, 99(21); Ferns et al., Oncoimmunology, 2015, 4(2); Suzuki et al., Lung Cancer, 2010, 67(3):361-365). Inhibition of IDO1 by Epacadostat improved anti-tumor efficacy of the anti-PD-1 checkpoint inhibitor Keytruda in a Phase II study in melanoma but development was terminated in Phase III (ECHO-301) because it lacked durable efficacy (Muller et al., Semin Immunopathol., 2019, 41(1):41-48). One possible explanation is that the tumors switched to other sources of AhR ligands that are not subject to Epacadostat inhibition.

Table 1 lists 29 AhR ligands that were investigated in the present disclosure as potential biomarkers predictive of a patient's cancer responsiveness to an ICI therapy.

TABLE 1

Piperine
nicotinamide
4-hydroxyphenylacetate
Urolithin A
Berberine
Quinolinate
Bilirubin
Phenylacetate
Citrulline
Phenylpyruvate
Xanthurenate
Spermidine
Indolepropionate
pyridoxine (Vitamin B6)
Indole
Indolelactate
Kynurenate
Curcumin
Biliverdin
Indoleacetate
Picolinate
Kynurenine
Tryptophan
3-indoxyl sulfate
Serotonin
phenyllactate (PLA)
Tryptamine
benzoate
4-hydroxyphenylpyruvate

Example 1 shows that among these 29 AhR ligands, kynurenine, tryptophan, phenylpyruvate, and indolelactate were differentially secreted in fecal samples at baseline with an FDR corrected p-value less than 0.05 when comparing between patients who became responsive to an ICI therapy (responders) and those who were not responsive to any ICI therapy (non-responders). All of these four AhR ligands were elevated in fecal samples from the non-responders compared to the responders. Furthermore, these four AhR ligands showed no statistical difference in plasma samples from the non-responders compared to the responders.

In some embodiments, the biomarker according to the disclosure comprises one or more of kynurenine, tryptophan, phenylpyruvate, and indolelactate. For example, in some embodiments, the biomarker according to the disclosure comprises kynurenine. In some embodiments, the biomarker according to the disclosure comprises tryptophan. In some embodiments, the biomarker according to the disclosure comprises phenylpyruvate. In some embodiments, the biomarker according to the disclosure comprises indolelactate.

In some embodiments, the biomarker according to the disclosure comprises kynurenine and tryptophan. In some embodiments, the biomarker according to the disclosure comprises kynurenine and phenylpyruvate. In some embodiments, the biomarker according to the disclosure comprises kynurenine and indolelactate. In some embodiments, the biomarker according to the disclosure comprises tryptophan and phenylpyruvate. In some embodiments, the biomarker according to the disclosure comprises tryptophan and indolelactate. In some embodiments, the biomarker according to the disclosure comprises phenylpyruvate and indolelactate.

In some embodiments, the biomarker according to the disclosure comprises kynurenine, tryptophan, and phenylpyruvate. In some embodiments, the biomarker according to the disclosure comprises kynurenine, tryptophan, and indolelactate. In some embodiments, the biomarker according to the disclosure comprises tryptophan, phenylpyruvate, and indolelactate.

In some embodiments, the biomarker according to the disclosure comprises kynurenine, tryptophan, phenylpyruvate, and indolelactate.

The biomarkers according to the disclosure are predictive of a patient's cancer responsiveness to ICI therapy. Several ICI therapies have been approved by the Food and Drug Administration to treat, inter alia, melanoma, non-small cell lung cancer, renal cell carcinoma, head and neck squamous cell carcinoma, Hodgkin's lymphoma, urothelial carcinoma, small cell lung cancer, esophageal squamous cell carcinoma, cervical cancer, primary mediastinal large B-cell lymphoma, MSI-H/dMMR colorectal cancer, hepatocellular carcinoma, Merkel cell carcinoma, triple-negative breast cancer, and cutaneous squamous cell carcinoma.

In some embodiments, the ICI therapy comprises an agent that inhibits one or more of CTLA-4, PD-1, PD-L1, VISTA, B7-H2, B7-H3, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73, and A2aR.

CTLA-4 and PD-1 are negative regulators of T-cell immune function and act at different stages of the immune response. While CTLA-4 regulates early activation of naïve and memory T-cell, typically within lymph nodes, the PD-1 pathway regulates previously activated T cells in response to an inflammatory signal at later stages of an immune response, primarily in peripheral tissues and within the cancer itself (Fife et al., Immunol Rev., 2008, 224:166-182).

PD-1 is highly expressed by activated T cells, B cells, dendritic cells (DC), and natural killer cells (NK), whereas PD-L1 can be expressed on several types of tumor cells. PD-1 is regulated by two ligands, programmed death ligand 1 (PD-L1; B7-H1, CD274) and PD-L2 (B7-DC), resulting in the inhibition of T-cell activation. PD-1 binding inhibits T-cell proliferation and the production of proinflammatory cytokines like interferon-γ (IFN-γ), tumor necrosis factor-α, and IL-2 (Chamoto et al., Curr Top Microbiol Immunol., 2017, 410:75-97). The ligation of PD-L1 with PD-1 in normal tissues maintains homeostasis of the immune system and prevents autoimmunity during infection or inflammation. Their interaction in tumor microenvironment provides an immune escape mechanism for tumor cells by turning off cytotoxic T cells, which seemingly blocks these interactions.

CTLA-4, PD-1, and PD-L1 are well-validated targets for ICI therapies. Approved ICI therapies targeting these checkpoint proteins include Pembrolizumab (Keytruda), Nivolumab (Opdivo), Ipilimumab (Yervoy), Avelumab (Bavencio), Atezolizumab (Tecentriq), Durvalumab (Imfinzi), Cemiplimab (LBTAYO), Sintilimab (Tyvyt), Toripalimab (Tuoyi), and Camrelizumab (AiRuiKa).

Ongoing clinical trials include therapeutic agents targeting LAG-3, TIM-3 TIGIT, and VISTA (Qin et al. Molecular Cancer, 2019, 18:155). Non-limiting examples of ICI therapy targeting LAG-3 include IMP321 (Eftilagimod alpha), Relatlimab (BMS-986016), LAG525, MK-4280, REGN3767, TSR-033, BI754111, Sym022, FS118, and MGD013. Non-limiting examples of ICI therapy targeting TIM-3 include TSR-022, MBG453, Sym023, INCAGN2390, LY3321367, BMS-986258, SHR-1702, and RO7121661. Non-limiting examples of ICI therapy targeting TIGIT include MK-7684, Etigilimab (OMP-313), Tiragolumab (MTIG7192A, RG-6058), BMS-986207, AB-154, and ASP-8374. Non-limiting examples of ICI therapy targeting VISTA include JNJ-61610588 and CA-170.

In some embodiments, the ICI therapy according to the disclosure comprises an anti-PD-1 antibody, an anti-PD-L1 antibody, an anti-CTLA-4 antibody, an anti-LAG-3 antibody, an anti-TIM-3 antibody, an anti-TIGIT antibody, an anti-VISTA antibody, or combinations thereof.

In some embodiments, the ICI therapy according to the disclosure comprises Pembrolizumab (Keytruda), Nivolumab (Opdivo), Ipilimumab (Yervoy), Avelumab (Bavencio), Atezolizumab (Tecentriq), Durvalumab (Imfinzi), Cemiplimab (LBTAYO), Sintilimab (Tyvyt), Toripalimab (Tuoyi), Camrelizumab (AiRuiKa), IMP321 (Eftilagimod alpha), Relatlimab (BMS-986016), LAG525, MK-4280, REGN3767, TSR-033, BI754111, Sym022, FS118, MGD013, TSR-022, MBG453, Sym023, INCAGN2390, LY3321367, BMS-986258, SHR-1702, RO7121661, MK-7684, Etigilimab (OMP-313), Tiragolumab (MTIG7192A, RG-6058), BMS-986207, AB-154, ASP-8374, JNJ-61610588, CA-170, or combinations thereof.

According to the present disclosure, the amount of the biomarker in a biological sample is measured using one or more applicable methods known in the art. In some embodiments, the biological sample comprises a plasma sample taken from a cancer patient. In some embodiments, the biological sample comprises a serum, plasma, or fecal sample (e.g., an intestinal luminal sample) taken from a cancer patient.

### Diagnostic/Prognostic and Therapeutic Uses of Biomarkers

The biomarkers according to the disclosure can be used in a variety of diagnostic/prognostic and therapeutic applications, or combinations thereof.

The biomarkers according to the disclosure can be used in in vitro methods of predicting a patient's cancer responsiveness to an ICI therapy. In some embodiments, the method comprises (a) measuring or having measured an amount of at least one biomarker from a serum, plasma, or fecal sample obtained from the patient, (b) comparing or having compared the amount of the at least one biomarker to a threshold score, and (c) determining that the patient's cancer is likely to be responsive to an ICI therapy if the amount of the at least one biomarker is equal to or below the threshold score.

The biomarkers according to the disclosure can be used in in vitro methods of determining whether a patient would benefit from being treated with an AhR antagonist. In some embodiments, the method comprises (a) measuring or having measured an amount of at least one biomarker from a serum, plasma, or fecal sample obtained from the patient, (b) comparing or having compared the amount of the at least one biomarker to a threshold score, and (c) determining that the patient would benefit from being treated with an AhR antagonist if the amount of the at least one biomarker is above the threshold score.

The biomarkers according to the disclosure may be used to stratify cancer patients based on the amount of at least one biomarker present in the cancer patient's serum, plasma, or fecal sample. In some embodiments, such stratification may be used to inform and provide an effective therapeutic regimen for treating cancer patients.

In some embodiments, when the amount of the at least one biomarker in a patient's serum, plasma, or fecal sample is equal to or below the threshold score for that biomarker, the patient is likely to be responsive to an ICI therapy even without the ICI therapy being administered in combination with an AhR antagonist. In such embodiments, the patient may be treated with an ICI therapy disclosed herein. Therefore, in some embodiments, the present disclosure provides a method of treating cancer in a patient in need thereof, comprising: (a) measuring or having measured an amount of at least one biomarker from a serum, plasma, or fecal sample obtained from the patient, (b) comparing or having compared the amount of the at least one AhR ligand to a threshold score, (c) treating the patient with an ICI therapy if the amount of the at least one biomarker is equal to or below the threshold score. In some embodiments, the ICI therapy may be optionally combined with a different cancer therapy known in the art, such as surgery to remove cancerous tissues, chemotherapy, or radiation therapy. In some embodiments, the different cancer therapy is not an AhR antagonist. In some embodiments, the different cancer therapy is an AhR antagonist. In some embodiments, the at least one AhR ligand is chosen from piperine, nicotinamide, 4-hydroxyphenylacetate, urolithin A, berberine, quinolinate, bilirubin, phenylacetate, citrulline, phenylpyruvate, xanthurenate, spermidine, indolepropionate, pyridoxine (Vitamin B6), indole, indolelactate, kynurenate, curcumin, biliverdin, indoleacetate, picolinate, kynurenine, tryptophan, 3-indoxyl sulfate, serotonin, phenyllactate (PLA), tryptamine, benzoate, and 4-hydroxyphenylpyruvate. In some embodiments, the at least one AhR ligand comprises two or more of piperine, nicotinamide, 4-hydroxyphenylacetate, urolithin A, berberine, quinolinate, bilirubin, phenylacetate, citrulline, phenylpyruvate, xanthurenate, spermidine, indolepropionate, pyridoxine (Vitamin B6), indole, indolelactate, kynurenate, curcumin, biliverdin, indoleacetate, picolinate, kynurenine, tryptophan, 3-indoxyl sulfate, serotonin, phenyllactate (PLA), tryptamine, benzoate, and 4-hydroxyphenylpyruvate. In some embodiments, the at least one AhR ligand comprises three or more of piperine, nicotinamide, 4-hydroxyphenylacetate, urolithin A, berberine, quinolinate, bilirubin, phenylacetate, citrulline, phenylpyruvate, xanthurenate, spermidine, indolepropionate, pyridoxine (Vitamin B6), indole, indolelactate, kynurenate, curcumin, biliverdin, indoleacetate, picolinate, kynurenine, tryptophan, 3-indoxyl sulfate, serotonin, phenyllactate (PLA), tryptamine, benzoate, and 4-hydroxyphenylpyruvate. In some embodiments, the at least one AhR ligand comprises kynurenine, tryptophan, phenylpyruvate, indolelactate, or combinations thereof.

In some embodiments, when the amount of the at least one biomarker in a patient's serum, plasma, or fecal sample is above the threshold score for that biomarker, the patient is likely to be non-responsive to an ICI therapy in the absence of an AhR antagonist (e.g., an ICI therapy only or an ICI therapy in combination with a different (non-AhR antagonist) cancer therapy, such as surgery, chemotherapy, and/or radiation therapy). In such embodiments, the patient may be treated with an ICI therapy in combination with an AhR antagonist. Therefore, in some embodiments, the present disclosure provides a method of treating cancer in a patient in need thereof, comprising: (a) measuring or having measured an amount of at least one biomarker from a serum, plasma, or fecal sample obtained from the patient, (b) comparing or having compared the amount of the at least one AhR ligand to a threshold score, (c) determining or having determined that the patient would benefit from being treated with an AhR antagonist if the amount of the at least one biomarker is above the threshold score, and (d) treating the patient determined to benefit in (d) with an effective amount of the AhR antagonist.

In some embodiments, the present disclosure provides a method of treating cancer in a patient in need thereof with a combination therapy comprising an ICI therapy and an AhR antagonist, comprising: (a) measuring or having measured an amount of at least one biomarker from a serum, plasma, or fecal sample obtained from the patient, (b) comparing or having compared the amount of the at least one biomarker to a threshold score, (c) determining or having determined that the patient would benefit from the combination therapy if the amount of the at least one biomarker is above the threshold score, and (d) treating the patient determined to benefit in (c) with the combination therapy.

In some embodiments of the diagnostic/prognostic and therapeutic methods disclosed herein, the biomarker is chosen from piperine, nicotinamide, 4-hydroxyphenylacetate, urolithin A, berberine, quinolinate, bilirubin, phenylacetate, citrulline, phenylpyruvate, xanthurenate, spermidine, indolepropionate, pyridoxine (Vitamin B6), indole, indolelactate, kynurenate, curcumin, biliverdin, indoleacetate, picolinate, kynurenine, tryptophan, 3-indoxyl sulfate, serotonin, phenyllactate (PLA), tryptamine, benzoate, and 4-hydroxyphenylpyruvate. For example, in some embodiments, the biomarker comprises two or more of piperine, nicotinamide, 4-hydroxyphenylacetate, urolithin A, berberine, quinolinate, bilirubin, phenylacetate, citrulline, phenylpyruvate, xanthurenate, spermidine, indolepropionate, pyridoxine (Vitamin B6), indole, indolelactate, kynurenate, curcumin, biliverdin, indoleacetate, picolinate, kynurenine, tryptophan, 3-indoxyl sulfate, serotonin, phenyllactate (PLA), tryptamine, benzoate, and 4-hydroxyphenylpyruvate. In some embodiments, the biomarker comprises three or more of piperine, nicotinamide, 4-hydroxyphenylacetate, urolithin A, berberine, quinolinate, bilirubin, phenylacetate, citrulline, phenylpyruvate, xanthurenate, spermidine, indolepropionate, pyridoxine (Vitamin B6), indole, indolelactate, kynurenate, curcumin, biliverdin, indoleacetate, picolinate, kynurenine, tryptophan, 3-indoxyl sulfate, serotonin, phenyllactate (PLA), tryptamine, benzoate, and 4-hydroxyphenylpyruvate.

In some embodiments of the diagnostic/prognostic and therapeutic methods disclosed herein, the biomarker comprises kynurenine, tryptophan, phenylpyruvate, indolelactate, or combinations thereof. For example, in some embodiments, the biomarker comprises kynurenine. In some embodiments, the biomarker comprises tryptophan. In some embodiments, the biomarker comprises phenylpyruvate. In some embodiments, the biomarker comprises indolelactate. In some embodiments, the biomarker comprises kynurenine and tryptophan. In some embodiments, the biomarker comprises kynurenine and phenylpyruvate. In some embodiments, the biomarker comprises kynurenine and indolelactate. In some embodiments, the biomarker comprises tryptophan and phenylpyruvate. In some embodiments, the biomarker comprises tryptophan and indolelactate. In some embodiments, the biomarker comprises phenylpyruvate and indolelactate. In some embodiments, the biomarker comprises kynurenine, tryptophan, and phenylpyruvate. In some embodiments, the biomarker comprises kynurenine, tryptophan, and indolelactate. In some embodiments, the biomarker comprises tryptophan, phenylpyruvate, and indolelactate. In some embodiments, the biomarker comprises kynurenine, tryptophan, phenylpyruvate, and indolelactate.

In some embodiments of the diagnostic/prognostic and therapeutic methods disclosed herein, the cancer patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

In some embodiments of the diagnostic/prognostic and therapeutic methods disclosed herein, the cancer patient has been previously treated with an ICI therapy but is not responding to the ICI therapy. In some embodiments, the cancer patient has been previously treated with an ICI therapy and has developed resistance to the ICI therapy. In some embodiments, the ICI therapy comprises an anti-PD-1 antibody, an anti-PD-L1 antibody, an anti-CTLA-4 antibody, an anti-LAG-3 antibody, an anti-TIM-3 antibody, an anti-TIGIT antibody, an anti-VISTA antibody, or combinations thereof. In some embodiments, the ICI therapy comprises Pembrolizumab (Keytruda), Nivolumab (Opdivo), Ipilimumab (Yervoy), Avelumab (Bavencio), Atezolizumab (Tecentriq), Durvalumab (Imfinzi), Cemiplimab (LBTAYO), Sintilimab (Tyvyt), Toripalimab (Tuoyi), Camrelizumab (AiRuiKa), IMP321 (Eftilagimod alpha), Relatlimab (BMS-986016), LAG525, MK-4280, REGN3767, TSR-033, BI754111, Sym022, FS118, MGD013, TSR-022, MBG453, Sym023, INCAGN2390, LY3321367, BMS-986258, SHR-1702, RO7121661, MK-7684, Etigilimab (OMP-313), Tiragolumab (MTIG7192A, RG-6058), BMS-986207, AB-154, ASP-8374, JNJ-61610588, CA-170, or combinations thereof.

In some embodiments of the diagnostic/prognostic and therapeutic methods disclosed herein, the cancer patient may have been previously treated with, or is currently on, a different (non-ICI and non-AhR antagonist) cancer therapy such as surgery removing cancerous tissues, chemotherapy, or radiation therapy.

In some embodiments of the diagnostic/prognostic and therapeutic methods disclosed herein, the amount of the biomarker disclosed herein is measured in a serum, plasma, or fecal sample taken from a cancer patient at an appropriate time. In some embodiments, the sample is taken approximately one week following initiation of an ICI therapy. In some embodiments, the sample is taken approximately two weeks following initiation of an ICI therapy. In some embodiments, the sample is taken approximately three weeks following the initiation of an ICI therapy. In some embodiments, the sample is taken approximately four weeks following the initiation of an ICI therapy. In some embodiments, the sample is taken approximately five weeks following the initiation of an ICI therapy. In some embodiments, the sample is taken approximately six weeks following the initiation of an ICI therapy. In some embodiments, the sample is taken approximately seven weeks following the initiation of an ICI therapy. In some embodiments, the sample is taken approximately eight weeks following the initiation of an ICI therapy. In some embodiments, the sample is taken approximately nine weeks following the initiation of an ICI therapy. In some embodiments, the sample is taken approximately ten weeks following the initiation of an ICI therapy. In some embodiments, the sample is taken more than ten weeks, three months, five months, seven months, ten months, one year, two years, three years, four years, or five years following the initiation of an ICI therapy.

Sample preparation can involve any procedures known in the art. Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, and extraction and purification of lipids.

After sample preparation, the amount of the biomarker in a serum, plasma, or fecal sample is measured using one or more applicable methods known in the art. Suitable measuring/detection methods may include a mass spectrometry method, such as liquid chromatography mass spectrometry (LC-MS), gas-phase chromatography mass spectrometry (GC-MS), or tandem mass spectrometry (MS-MS). Other suitable methods include reversed-phase chromatography (e.g., with positive and/or negative ionization mode) and hydrophobic interaction liquid ion chromatography (HILIC) (e.g., with positive and/or negative ionization mode), or a combination thereof. In some embodiments, the amount of the biomarker is measured by LC-MS. In some embodiments, the amount of the biomarker is analyzed using ultrahigh performance liquid chromatography-tandem mass spectroscopy. See Example 1.

In some embodiments, the fecal sample may be fractionated prior to application of the measuring/detection method. In some embodiments, the amount of the biomarker may be measured by methods that do not require physical separation of the biomarkers themselves. For example, nuclear magnetic resonance (NMR) spectroscopy may be used to resolve the amount of a biomarker from a complex mixture of metabolites including various AhR ligands.

In some embodiments of the diagnostic/prognostic and therapeutic methods disclosed herein, the amount of each biomarker is compared to a predetermined threshold score for that biomarker. In some embodiments, the threshold score is the amount of the biomarker in a serum, plasma, or fecal sample from a control cancer patient who is responding to an ICI therapy. In some embodiments, the threshold score can be generated from a population or cohort of two or more control cancer patients who are responding to ICI therapies. The population or cohort, for example, may comprise at least 2, 3, 4, 5, 10, 15, 18, 20, 30, 40, 50, 75, 100 or more cancer patients. In such embodiments, the threshold score for each biomarker may be the average amount of that biomarker in serum, plasma, or fecal samples from a population or cohort of control cancer patients who are responding to ICI therapies.

To determine the threshold score for each biomarker, the serum, plasma, or fecal samples may be taken from control cancer patients who are responding to ICI therapies at an appropriate time, such as those disclosed herein. The control cancer patient may be responding to an ICI therapy comprising any agent that inhibits one or more of CTLA-4, PD-1, PD-L1, VISTA, B7-H2, B7-H3, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73, and A2aR. In some embodiments, the ICI therapy comprises an anti-PD-1 antibody, an anti-PD-L1 antibody, an anti-CTLA-4 antibody, an anti-LAG-3 antibody, an anti-TIM-3 antibody, an anti-TIGIT antibody, an anti-VISTA antibody, or combinations thereof. In some embodiments, the ICI therapy comprises Pembrolizumab (Keytruda), Nivolumab (Opdivo), Ipilimumab (Yervoy), Avelumab (Bavencio), Atezolizumab (Tecentriq), Durvalumab (Imfinzi), Cemiplimab (LBTAYO), Sintilimab (Tyvyt), Toripalimab (Tuoyi), Camrelizumab (AiRuiKa), IMP321 (Eftilagimod alpha), Relatlimab (BMS-986016), LAG525, MK-4280, REGN3767, TSR-033, BI754111, Sym022, FS118, MGD013, TSR-022, MBG453, Sym023, INCAGN2390, LY3321367, BMS-986258, SHR-1702, RO7121661, MK-7684, Etigilimab (OMP-313), Tiragolumab (MTIG7192A, RG-6058), BMS-986207, AB-154, ASP-8374, JNJ-61610588, CA-170, or combinations thereof.

The amount of a biomarker in the serum, plasma, or fecal sample from a cancer patient may be “above” the threshold score for that biomarker, for example, at least 10%, 15%, 20%, 30%, 40%, 50%, 60%, 70%, 80%, 90%, 100%, 150%, 200%, 300%, 350%, 400%, 500%, 600%, 700%, 800%, 900%, 1000% or more higher than the threshold score. In some embodiments, the amount of a biomarker in the serum, plasma, or fecal sample from a test cancer patient may be “below” the threshold score for that biomarker, for example, at least about 10%, 15%, 20%, 30%, 40%, 50%, 60%, 70%, 80%, 90%, or more lower than the threshold score.

In some embodiments of the therapeutic methods disclosed herein, when the amount of a biomarker in a cancer patient's serum, plasma, or fecal sample is above the threshold score, the patient may be administered an AhR antagonist in combination with an ICI therapy. In these embodiments, the AhR antagonist may be administered simultaneously with the ICI therapy. In some embodiments, the AhR antagonist is administered sequentially with the ICI therapy. In some embodiments, the combination therapy results in an increased or synergistic anti-tumor response compared to a treatment using the corresponding ICI therapy alone.

### Pyridopyrimidinone Derivatives as AhR Antagonists

Disclosed herein are novel 6,8-trisubstituted pyrido[3,4-d]pyrimidin-4(3H)-one compounds of formula (I), formula (Ia), or formula (Ib), or pharmaceutically acceptable salts thereof, that effectively inhibit AhR and therefore, like known AhR antagonists, can be used in the therapeutic methods disclosed herein.

Unless indicated otherwise, nomenclature used to describe chemical groups or moieties as used herein follow the convention in which, reading the name from left to right, the point of attachment to the rest of the molecule is at the right-hand side of the name. For example, the group “(C₁₋₃ alkoxy)C₁₋₃ alkyl,” is attached to the rest of the molecule at the alkyl end. Further examples include methoxyethyl, where the point of attachment is at the ethyl end, and methylamino, where the point of attachment is at the amine end.

Unless indicated otherwise, when a chemical group is described by its chemical formula or structure having a terminal bond moiety indicated by “—,” it will be understood that the “—” represents the point of attachment. In some embodiments, a wavy line (i.e., [symbol] ) depicts the point of attachment.

As used herein, the term “pharmaceutically acceptable salt” refers to a salt that is pharmaceutically acceptable as defined herein and that has the desired pharmacological activity of the parent compound. Non-limiting examples of pharmaceutically acceptable salts include those derived from inorganic acids, non-limiting examples of which include hydrochloric acid, hydrobromic acid, hydroiodic acid, sulfuric acid, nitric acid, and phosphoric acid, and those derived from organic acids, non-limiting examples of which include acetic acid, trifluoroacetic acid, propionic acid, glycolic acid, pyruvic acid, oxalic acid, stearic acid, malic acid, maleic acid, malonic acid, salicylic acid, succinic acid, fumaric acid, tartaric acid, citric acid, benzoic acid, cinnamic acid, mandelic acid, p-toluenesulfonic acid, methanesulfonic acid, ethanesulfonic acid, and lactic acid. Additional non-limiting examples of pharmaceutically acceptable salts include those formed when an acidic proton in a parent compound is replaced by a metal ion, non-limiting examples of which include an alkali metal ion and an alkaline earth metal ion, and those formed when an acidic proton present in a parent compound is replaced by a ammonium ion, a primary ammonium ion, a secondary ammonium ion, a tertiary ammonium ion, or a quaternary ammonium ion. Non-limiting examples of alkali metals and alkaline earth metals include sodium, potassium, lithium, calcium, aluminum, magnesium, copper, zinc, iron, and manganese. Additional non-limiting examples of pharmaceutically acceptable salts include those comprising one or more counterions and zwitterions.

As used herein, an “acyl” or “alkanoyl” is a functional group with formula RCO— where R is bound to the carbon atom of the carbonyl functional group by a single bond and the “—” denotes the point of attachment to the rest of the molecule. Non-limiting examples of acyls include formyl (HC(O)—, also called methanoyl), acetyl (CH₃C(O)—, also called ethanoyl), and benzoyl (PhC(O)—).

The term “alkyl” or “aliphatic” as used herein, means a straight-chain (i.e., unbranched) or branched, substituted or unsubstituted hydrocarbon chain that is completely saturated and that has a single point of attachment to the rest of the molecule. Unless otherwise specified, an alkyl group is a hydrocarbon chain of 1 to 20 alkyl carbon atoms. In some embodiments, an alkyl group contains one to twelve carbon atoms (C₁-C₁₂). In some embodiments, an alkyl group contains one to eight carbon atoms (C₁-C₈). In some embodiments, an alkyl group contains one to six carbon atoms (C₁-C₆). In some embodiments, an alkyl group contains one to four carbon atoms (C₁-C₄). In some embodiments, a cyclic alkyl group contains three to six carbon atoms (C₃-C₆). Non-limiting examples of substituted and unsubstituted linear, branched, and cyclic alkyl groups include methyl, ethyl, n-propyl, iso-propyl, cyclopropyl, n-butyl, sec-butyl, iso-butyl, tert-butyl, cyclobutyl, cyclopentyl, cyclohexyl, hydroxymethyl, chloromethyl, fluoromethyl, trifluoromethyl, aminomethyl, 2-aminoethyl, 3-aminopropyl, 4-aminobutyl, dimethylaminomethyl, 2-dimethylaminoethyl, 3-dimethylaminopropyl, 4-dimethylaminobutyl, cyclopropylmethyl, cyclobutylmethyl, cyclopentylmethyl, trifluoroethyl, and trifluoropropyl.

“Alkoxy,” as used herein, refers to an alkyl group, as previously defined, attached to the principal carbon chain through an oxygen (“alkoxy”) atom.

“Halo” and “halogen,” as used herein, are interchangeable and refer to halogen atoms such as fluoro (F), chloro (Cl), bromo (Br), and iodo (I).

“Haloalkyl” refers to an alkyl group substituted with one or more halo atoms (F, Cl, Br, I). For example, “fluoromethyl” refers to a methyl group substituted with one or more fluoro atoms (e.g., monofluoromethyl, difluoromethyl, or trifluoromethyl).

“Haloalkoxy” refers to an alkoxy group substituted with one or more halo atoms (F, Cl, Br, I). For example, “fluoromethoxy” refers to a methoxy group substituted with one or more fluoro atoms (e.g., monofluoromethoxy, difluoromethoxy, or trifluoromethoxy).

“Hydroxyalkyl” refers to an alkyl group substituted with one or more hydroxy groups (—OH).

The terms “cycloalkyl” and “cycloalkyl group” as used interchangeably herein refer to a cyclic saturated monovalent hydrocarbon radical of three to twelve carbon atoms that has a single point of attachment to the rest of the molecule. Cycloalkyl groups may be unsubstituted or substituted. In some embodiments, a cycloalkyl group comprises three to eight carbon atoms (C₃-C₈). In some embodiments, a cycloalkyl group comprises three to six carbon atoms (C₃-C₆). Non-limiting examples of substituted and unsubstituted cycloalkyls include cyclopropyl, cyclopropylmethyl, cyclobutyl, cyclobutylmethyl, cyclopentyl, cyclopentylmethyl, cyclohexyl, cycloheptyl, and cyclooctyl.

The terms “alkylene” and “alkylene group” as used interchangeably herein refer to a saturated divalent (i.e., having two points of attachment to the rest of the molecule) hydrocarbon radical comprising one to twelve carbon atoms (C₁-C₁₂). Alkylene groups may be linear, branched, or cyclic. Alkylene groups may be unsubstituted or substituted. In some embodiments, an alkylene group comprises one to eight carbon atoms (C₁-C₈). In some embodiments, an alkylene group comprises one to six carbon atoms (C₁-C₆). In some embodiments, an alkylene group comprises one to four carbon atoms (C₁-C₄). Non-limiting examples of alkylene groups include methylene and ethylene.

The terms “alkenyl” and “alkenyl group” as used interchangeably herein refer to a monovalent (i.e., having a single point of attachment to the rest of the molecule) hydrocarbon radical comprising two to eight carbon atoms (C₂-C₈) with at least one site of unsaturation (i.e., an sp2 carbon-carbon double bond). Alkenyl groups may be linear, branched, or cyclic. Alkenyl groups may be unsubstituted or substituted. In some embodiments, an alkenyl group contains two to six carbon atoms (C₂-C₆). In some embodiments, an alkenyl group contains two to four carbon atoms (C₂-C₄). Alkenyl groups may have E or Z orientations. Non-limiting examples of alkenyl groups include ethenyl (also called vinyl), 1-propenyl, iso-propenyl, and 2-chloroethenyl.

The terms “alkenylene” and “alkenylene group” as used interchangeably herein refer to a divalent (i.e., having two points of attachment to the rest of the molecule) hydrocarbon radical of two to eight carbon atoms (C₂-C₈) with at least one site of unsaturation (e.g., an sp2 carbon-carbon double bond). Alkenylene groups may be linear, branched, or cyclic. Alkenylene groups may be unsubstituted or substituted. In some embodiments, an alkylene group contains two to six carbon atoms (C₂-C₆). In some embodiments, an alkylene group contains two to four carbon atoms (C₂-C₄). Alkylene groups may have E or Z orientations. A non-limiting example of an alkenyl group is ethenylene (also called vinylene).

The terms “alkynyl” and “alkynyl group” as used interchangeably herein refer to a monovalent (i.e., having a single point of attachment to the rest of the molecule) hydrocarbon radical of two to eight carbon atoms (C₂-C₈) with at least one site of unsaturation (i.e., an sp carbon-carbon triple bond). Alkynyl groups may be linear or branched. Alkynyl groups may be unsubstituted or substituted. In some embodiments, an alkynyl group contains two to six carbon atoms (C₂-C₆). In some embodiments, an alkynyl group contains two to four carbon atoms (C₂-C₄). A non-limiting example of an alkynyl group is ethynyl.

The terms “alkynylene” and “alkynylene group” as used interchangeably herein refer to a divalent (i.e., having two points of attachment to the rest of the molecule) hydrocarbon radical of two to eight carbon atoms (C₂-C₈) with at least one site of unsaturation (i.e., an sp carbon-carbon triple bond). Alkynylene groups may be linear or branched. Alkynylene groups may be unsubstituted or substituted. In some embodiments, an alkynylene group contains two to six carbon atoms (C₂-C₆). In some embodiments, an alkynylene group contains two to four carbon atoms (C₂-C₄). A non-limiting example of an alkynylene group is ethynylene.

As used herein, “aromatic groups” or “aromatic rings” refer to chemical groups that contain conjugated, planar ring systems with delocalized pi electron orbitals comprised of [4n+2] p orbital electrons, wherein n is an integer ranging from 0 to 6. Non-limiting examples of aromatic groups include aryl and heteroaryl groups.

The terms “aryl” and “aryl group” as used interchangeably herein refer to a monovalent (i.e., having a single point of attachment to the rest of the molecule) aromatic hydrocarbon radical of 6-20 carbon atoms (C₆-C₂₀). Aryl groups can be unsubstituted or substituted. Non-limiting examples of unsubstituted and substituted aryl groups include phenyl, 2-fluorophenyl, 3-fluorophenyl, 4-fluorophenyl, 2-methylphenyl, 3-methylphenyl, 4-methylphenyl, 2-chlorophenyl, 3-chlorophenyl, 4-chlorophenyl, 2,6-dichlorophenyl, 3,4-difluorophenyl, 2-hydroxyphenyl, 3-hydroxyphenyl, 4-hydroxyphenyl, 2-methoxyphenyl, 3-methoxyphenyl, 4-methoxyphenyl, 2-phenoxyphenyl, 3-phenoxyphenyl, 4-phenoxyphenyl, 2-cyanophenyl, 3-cyanophenyl, 4-cyanophenyl, 2-dimethylaminophenyl, 3-dimethylaminophenyl, 4-dimethylaminophenyl, 3-methylsulfonylphenyl, 4-methylsulfonylphenyl, 3-aminophenyl, 3-methylaminophenyl, 3-(2-hydroxyethoxy)phenyl, 2-trifluoromethylphenyl, 3-trifluoromethylphenyl, 4-trifluoromethylphenyl, 2-isopropylphenyl, 3-isopropylphenyl, 4-isopropylphenyl, 1-naphthyl and 2-naphthyl.

The term “heteroalkyl” as used herein refers to an alkyl group wherein at least one of the carbon atoms in the chain is replaced by a heteroatom, such as nitrogen, oxygen, phosphorous, and sulfur. A heteroalkyl group may be unsubstituted or substituted.

The terms “heterocycloalkyl,” “heterocycle,” “heterocyclyl,” and “heterocyclic group” as used interchangeably herein refer to a saturated or partially unsaturated ring system of 3 to 20 atoms, wherein at least one of the ring atoms is a heteroatom, such as nitrogen, oxygen, phosphorous, and sulfur. A heterocycloalkyl group may be unsubstituted or substituted. In some embodiments, a heterocycloalkyl group comprises 3 to 10 atoms. In some embodiments, a heterocycloalkyl group contains 3 to 7 atoms. In some embodiments, a heterocycloalkyl group is monocyclic. In some embodiments, a heterocycloalkyl group is bicyclic. In some embodiments, a heterocycloalkyl group comprises fused rings. Non-limiting examples of unsubstituted and substituted heterocycloalkyl groups include pyrrolidinyl, N-methylpyrrolidinyl, azetidinyl, dihydrofuranyl, tetrahydrofuranyl, tetrahydropyranyl, 3-hydroxypyrrolidinyl, 3-methoxypyrrolidinyl, and benzodioxolyl.

The terms “heteroaryl” and “heteroaryl group” as used interchangeably herein refer to an aromatic ring system of 3 to 20 atoms, wherein at least one of the ring atoms is a heteroatom, such as nitrogen, oxygen, phosphorous, and sulfur. A heteroaryl group may be unsubstituted or substituted. In some embodiments, a heteroaryl group contains 5 to 20 atoms. In some embodiments, a heteroaryl group contains 5 to 9 atoms. In some embodiments, a heteroaryl group contains 5 atoms. In some embodiments, a heteroaryl group contains 6 atoms. In some embodiments, a heteroaryl group contains 7 atoms. In some embodiments, a heteroaryl group is monocyclic. In some embodiments, a heteroaryl group is bicyclic. In some embodiments, a heteroaryl group contains fused rings. Non-limiting examples of heteroaryl groups include pyridinyl, imidazolyl, imidazopyridinyl, pyrimidinyl, pyrazolyl, triazolyl, pyrazinyl, tetrazolyl, furyl, 2-thienyl, 3-thienyl, isoxazolyl, thiazolyl, oxadiazolyl, 3-methyl-1,2,4-oxadiazolyl, 3-phenyl-1,2,4-oxadiazolyl, oxazolyl, isothiazolyl, pyrrolyl, quinolinyl, isoquinolinyl, tetrahydroisoquinolinyl, indolyl, benzimidazolyl, benzofuranyl, indazolyl, indolizinyl, phthalazinyl, pyridazinyl, triazinyl, thiadiazolyl, furazanyl, benzofurazanyl, benzothiophenyl, benzothiazolyl, benzoxazolyl, quinazolinyl, quinoxalinyl, naphthyridinyl, furopyridinyl, and 1H-pyrrolo[2,3-b]pyridinyl. Non-limiting examples of heteroaryl groups include:

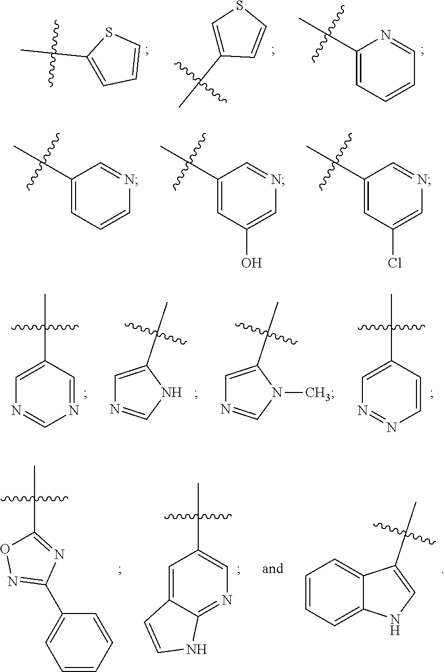

The phrase “optionally substituted” as used herein means that the specified component may or may not be “substituted.” The term “substituted” as used herein refers to the replacement of one or more hydrogen atoms on a group (such as on an alkyl group, alkylene group, alkenyl group, alkenylene group, alkynyl group, alkynylene group, aryl group, heterocycloalkyl group, or heteroaryl group) by one or more substituents. Non-limiting examples of substituents that replace a single hydrogen atom include halogen, hydroxyl, and amino. Non-limiting examples of substituents that replace two hydrogen atoms include oxo and methene. Non-limiting examples of substituents that replace three hydrogen atoms include nitrile.

Additional non-limiting examples of substituents include:
C₁-C₆ linear, branched, and cyclic alkyl groups, non-limiting examples of which include methyl, ethyl, n-propyl, iso-propyl, cyclopropyl, n-butyl sec-butyl, iso-butyl, tert-butyl, cyclobutyl, cyclopentyl, and cyclohexyl;
C₂-C₈ linear, branched, and cyclic alkenyl groups, non-limiting examples of which include ethenyl (also called vinyl), 1-propenyl, and iso-propenyl;
C₂-C₈ linear and branched alkynyl groups, non-limiting examples of which include ethynyl;
substituted and unsubstituted aryl groups, non-limiting examples of which include phenyl, 2-fluorophenyl, 3-methylphenyl, 4-chlorophenyl, 2,6-dichlorophenyl, 3,4-difluorophenyl, 3-hydroxyphenyl, 4-cyanophenyl, 2-dimethylaminophenyl, 3-methylsulfonylphenyl, 4-trifluoromethylphenyl, 3-isopropylphenyl, 1-naphthyl, and 2-naphthyl;
substituted and unsubstituted heterocyclic groups, non-limiting examples of which include pyrrolidinyl, N-methylpyrrolidinyl, azetidinyl, dihydrofuranyl, tetrahydrofuranyl, tetrahydropyranyl, 3-hydroxypyrrolidinyl, and 3-methoxypyrrolidinyl;
substituted and unsubstituted heteroaryl groups, non-limiting examples of which include pyridinyl, imidazolyl, pyrimidinyl, pyrazolyl, furyl, 2-thienyl, 3-thienyl, isoxazolyl, thiazolyl, oxadiazolyl, 3-methyl-1,2,4-oxadiazolyl, 3-phenyl-1,2,4-oxadiazolyl, indolyl, benzothiazolyl, and 1H-pyrrolo[2,3-b]pyridinyl;
—(CR^{a}R^{b})_{z}OR^{c}, non-limiting examples of which include —OH, —OCH₃, —OCH₂OH, and —OCH₂CH₃;
—(CR^{a}R^{b})_{z}N(R^{c})(R^{d}), non-limiting examples of which include —NH₂, —NHCH₃, —N(CH₃)₂,
—CH₂NH₂, —CH₂NHCH₃,
a halogen atom, non-limiting examples of which include a fluorine atom (—F) and a chlorine atom (—Cl);
—(CR^{a}R^{b})_{z}CN;
—(CR^{a}R^{b})_{z}NO₂;
—CHₓX_{y}, wherein X is a halogen atom and x+y sum to 3, non-limiting examples of which include —CH₂F, —CHF₂, and —CF₃;
—(CR^{a}R^{b})_{z}C(O)R^{c}, non-limiting examples of which include —COCH₃, —COCH₂CH₃, and —CH₂COCH₃;
—(CR^{a}R^{b})_{z}C(O)OR^{c}, non-limiting examples include CO₂H, —CO₂CH₃, —CO₂CH₂CH₃, and —CH₂CO₂CH₃,
—(CR^{a}R^{b})_{z}C(O)N(R^{c})(R^{d}), non-limiting examples of which include —CONH₂, —CONHCH₃, —CON(CH₃)₂, —CH₂CONH₂, —CH₂CONHCH₃, —CH₂CON(CH₃)₂;
(CR^{a}R^{b})_{z}SO₂R^{c}; non-limiting examples of which include —SO₂H, —SO₂CH₃, —CH₂SO₂H, —CH₂SO₂CH₃, —SO₂C₆H₅, and —CH₂SO₂C₆H₅; and
—(CR^{a}R^{b})_{z}SO₃R^{c}; non-limiting examples of which include —SO₃H, —SO₃CH₃, —CH₂SO₃H, —CH₂SO₃CH₃, —SO₃C₆H₅, and —CH₂SO₃C₆H₅;
wherein each of R^{a} and R^{b} is independently chosen from hydrogen and substituted or unsubstituted C₁-C₆ linear, branched, or cyclic alkyl, each of R^{c} and R^{d} is independently chosen from hydrogen, substituted or unsubstituted C₁-C₆ linear, branched, or cyclic alkyl, and aryl, or wherein R^{c} and R^{d} together form a ring system comprising 3 to 7 atoms, and z is chosen from 0, 1, 2, 3, and 4.

In some embodiments, an AhR antagonist according to the disclosure is a compound of Formula (I):

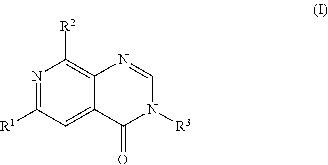

or a pharmaceutically acceptable salt thereof,
wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines, and optionally substituted heterocycloalkyls; and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

In some embodiments, R² is a dialkyl amine. In some embodiments, R² is a diethyl amine.

In some embodiments, an AhR antagonist according to the disclosure is a compound of Formula Ia:

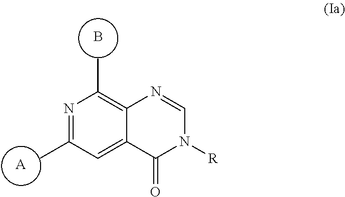

or a pharmaceutically acceptable salt thereof,
wherein:
ring A is chosen from optionally substituted aryls, optionally substituted heteroaryls, optionally substituted cycloalkyls, and optionally substituted heterocycloalkyls;
ring B is chosen from optionally substituted aryls, optionally substituted heteroaryls, optionally substituted cycloalkyls, and optionally substituted heterocycloalkyls; and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

In some embodiments, ring A is chosen from 6-10 membered aryls, 5-10 membered heteroaryls, 3-10 membered cycloalkyls, and 3-10 membered heterocycloalkyls, wherein each 6-10 membered aryl, 5-10 membered heteroaryl, 3-10 membered cycloalkyl, and 3-10 membered heterocycloalkyl is independently optionally substituted with 1 to 5 instances of R^{A}.

In some embodiments, ring B is chosen from 6-10 membered aryls, 5-10 membered heteroaryls, 3-10 membered cycloalkyls, and 3-10 membered heterocycloalkyls, wherein each 6-10 membered aryl, 5-10 membered heteroaryl, 3-10 membered cycloalkyl, and 3-10 membered heterocycloalkyl is independently optionally substituted with 1 to 5 instances of R^{B}.

In some embodiments, R is chosen from hydrogen, C₁-C₁₀ alkyls, 6-10 membered aryls, —C(O)R′, —C(O)NR′R′, 3-10 membered cycloalkyls, —C(O)OR′, C₁-C₁₀ heteroalkyls, 5-10 membered heteroaryls, 3-10 membered heterocycloalkyls, amino, cyano, halos, hydroxy, and —C(O)H, wherein each C₁-C₁₀ alkyl, 6-10 membered aryl, 3-10 membered cycloalkyl, C₁-C₁₀ heteroalkyl, 5-10 membered heteroaryl, and 3-10 membered heterocycloalkyl is independently optionally substituted with 1 to 5 instances of R^{C}.

In some embodiments, each R′ is independently chosen from hydrogen, C₁-C₁₀ alkyls, C₁-C₁₀ haloalkyls, C₁-C₁₀ hydroxyalkyls, and C₁-C₁₀ heteroalkyls.

In some embodiments, each R^{A} is independently chosen from halos, hydroxy, C₁-C₁₀ alkyls, C₁-C₁₀ haloalkyls, C₁-C₁₀ alkoxys, C₁-C₁₀ haloalkoxys, C₁-C₁₀ hydroxyalkyls, and NR″R″.

In some embodiments, each R^{B} is independently chosen from halos, hydroxy, C₁-C₁₀ alkyls, C₁-C₁₀ haloalkyls, C₁-C₁₀ alkoxys, C₁-C₁₀ haloalkoxys, C₁-C₁₀ hydroxyalkyls, and NR″R″.

In some embodiments, each R^{C} is independently chosen from halos, hydroxy, cyano, C₁-C₁₀ alkyls, C₁-C₁₀ alkoxys, C₁-C₁₀ haloalkyls, 3-10 membered cycloalkyls, 3-10 membered heterocycloalkyls, 6-10 membered aryls, and 5-10 membered heteroaryls.

In some embodiments, each R″ is independently chosen from hydrogen, C₁-C₁₀ alkyls, C₁-C₁₀ haloalkyls, C₁-C₁₀ hydroxyalkyls, and C₁-C₁₀ heteroalkyls.

In some embodiments, ring A is chosen from 6-10 membered aryls, 5-8 membered heteroaryls, 3-10 membered cycloalkyls, and 3-10 membered heterocycloalkyls, wherein each 6-10 membered aryl, 5-10 membered heteroaryl, 3-10 membered cycloalkyl, and 3-10 membered heterocycloalkyl is independently optionally substituted with 1 to 5 instances of R^{A};
ring B is chosen from 6-10 membered aryls, 5-10 membered heteroaryls, 3-10 membered cycloalkyls, and 3-10 membered heterocycloalkyls, wherein each 6-10 membered aryl, 5-10 membered heteroaryl, 3-10 membered cycloalkyl, and 3-10 membered heterocycloalkyl is independently optionally substituted with 1 to 5 instances of R^{B};
R is chosen from hydrogen, C₁-C₁₀ alkyls, 6-10 membered aryls, —C(O)R′, —C(O)NR′R′, 3-10 membered cycloalkyls, —C(O)OR′, C1-C10 heteroalkyls, 5-10 membered heteroaryls, 3-10 membered heterocycloalkyls, amino, cyano, halos, hydroxy, and —C(O)H, wherein each C₁-C₁₀ alkyl, 6-10 membered aryl, 3-10 membered cycloalkyl, C₁-C₁₀ heteroalkyl, 5-10 membered heteroaryl, and 3-10 membered heterocycloalkyl is independently optionally substituted with 1 to 5 instances of R^{C};
each R′ is independently chosen from hydrogen, C₁-C₁₀ alkyls, C₁-C₁₀ haloalkyls, C₁-C₁₀ hydroxyalkyls, and C₁-C₁₀ heteroalkyls;
each R^{A} is independently chosen from halos, hydroxy, C₁-C₁₀ alkyls, C₁-C₁₀ haloalkyls, C₁-C₁₀ alkoxys, C₁-C₁₀ haloalkoxys, C₁-C₁₀ hydroxyalkyls, and NR″R″;
each R^{B} is independently chosen from halos, hydroxy, C₁-C₁₀ alkyls, C₁-C₁₀ haloalkyls, C₁-C₁₀ alkoxys, C₁-C₁₀ haloalkoxys, C₁-C₁₀ hydroxyalkyls, and NR″R″;
each R^{C} is independently chosen from halos, hydroxy, cyano, C₁-C₁₀ alkyls, C₁-C₁₀ alkoxys, C₁-C₁₀ haloalkyls, 3-10 membered cycloalkyls, 3-10 membered heterocycloalkyls, 6-10 membered aryls, and 5-10 membered heteroaryls; and
each R″ is independently chosen from hydrogen, C₁-C₁₀ alkyls, C₁-C₁₀ haloalkyls, C₁-C₁₀ hydroxyalkyls, and C₁-C₁₀ heteroalkyls.

In some embodiments, ring A is chosen from 3-10 membered cycloalkyl optionally substituted with 1 to 5 instances of R^{A}. In some embodiments, ring A is chosen from cyclopropyl, cyclobutyl, cyclopentyl, cyclohexyl, cycloheptyl, and cyclooctyl optionally substituted with 1 to 5 instances of R^{A}. In some embodiments, ring A is chosen from 6-8 membered aryls optionally substituted with 1 to 5 instances of R^{A}. In some embodiments, ring A is phenyl optionally substituted with 1 to 3 instances of R^{A}. In some embodiments, ring A is chosen from 5-8 membered heteroaryls optionally substituted with 1 to 5 instances of R^{A}.

In some embodiments, ring A is chosen from pyrrolyl, furanyl, furazanyl, thiophenyl, imidazolyl, isothiazoyl, isoxazolyl, oxazolyl, oxadiazolyl, tetrazolyl, thiazolyl, triazolyl, pyrazolyl, pyridinyl, pyrazinyl, pyridazinyl, and pyrimidinyl, wherein each of pyrrolyl, furanyl, furazanyl, thiophenyl, imidazolyl, isothiazoyl, isoxazolyl, oxazolyl, oxadiazolyl, tetrazolyl, thiazolyl, triazolyl, pyrazolyl, pyridinyl, pyrazinyl, pyridazinyl, and pyrimidinyl is independently optionally substituted with 1 to 3 instances of R^{A}.

In some embodiments, ring A is pyridinyl optionally substituted with 1 to 3 instances of R^{A}. In some embodiments, ring A is chosen from 5-8 membered heterocycloalkyls optionally substituted with 1 to 5 instances of R^{A}. In some embodiments, ring A is chosen from pyrrolidinyl, pyrazolidinyl, piperidinyl, piperazinyl, morpholino, azepinyl, tetrahydropyranyl, and tetrahydrofuranyl, wherein each of pyrrolidinyl, pyrazolidinyl, piperidinyl, piperazinyl, morpholino, azepinyl, tetrahydropyranyl, and tetrahydrofuranyl is independently optionally substituted with 1 to 3 instances of R^{A}. In some embodiments, ring A is piperidinyl or morpholino optionally substituted with 1 to 3 instances of R^{A}.

In some embodiments, each R^{A} is independently chosen from halos, C₁-C₁₀ alkyls, C₁-C₁₀ haloalkyls, C₁-C₁₀ alkoxys, C₁-C₁₀ haloalkoxys, and NR″R″. In some embodiments,
each R^{B} is independently chosen from halos, C₁-C₁₀ alkyls, and C₁-C₁₀ haloalkyls. In some embodiments, each R^{C} is independently chosen from halos, hydroxy, cyano, C₁-C₁₀ alkyls, C₁-C₁₀ alkoxys, 3-8 membered cycloalkyls, 3-8 membered heterocycloalkyls, and 6-8 membered aryls. In some embodiments, each R″ is independently chosen from hydrogen and C₁-C₁₀ alkyls.

In some embodiments, each R^{A} is independently chosen from halos, C₁-C₁₀ alkyls, C₁-C₁₀ haloalkyls, C₁-C₁₀ alkoxys, C₁-C₁₀ haloalkoxys, and NR″R″;
each R^{B} is independently chosen from halos, C₁-C₁₀ alkyls, and C₁-C₁₀ haloalkyls;
each R^{C} is independently chosen from halos, hydroxy, cyano, C₁-C₁₀ alkyls, C₁-C₁₀ alkoxys, 3-8 membered cycloalkyls, 3-8 membered heterocycloalkyls, and 6-8 membered aryls; and
each R″ is independently chosen from hydrogen and C₁-C₁₀ alkyls.

In some embodiments, ring B is chosen from 6-8 membered aryls optionally substituted with 1 to 5 instances of R^{B}. In some embodiments, ring B is phenyl optionally substituted with 1 to 3 instances of R^{B}. In some embodiments, ring B is chosen from 5-8 membered heteroaryls optionally substituted with 1 to 5 instances of R^{B}. In some embodiments, ring B is chosen from pyrrolyl, furanyl, furazanyl, thiophenyl, imidazolyl, isothiazoyl, isoxazolyl, oxazolyl, oxadiazolyl, tetrazolyl, thiazolyl, triazolyl, pyrazolyl, pyridinyl, pyrazinyl, pyridazinyl, pyridinonyl, and pyrimidinyl, wherein each of pyrrolyl, furanyl, furazanyl, thiophenyl, imidazolyl, isothiazoyl, isoxazolyl, oxazolyl, oxadiazolyl, tetrazolyl, thiazolyl, triazolyl, pyrazolyl, pyridinyl, pyrazinyl, pyridazinyl, and pyrimidinyl is independently optionally substituted with 1 to 3 instances of R^{B}. In some embodiments, ring B is chosen from pyrazolyl, isothiazoyl, isoxazolyl, pyridinyl, pyrimidinyl, and thiophenyl, wherein each of pyrazolyl, isothiazoyl, isoxazolyl, pyridinyl, pyrimidinyl, and thiophenyl is independently optionally substituted with 1 to 3 instances of R^{B}.

In some embodiments, ring A is chosen from

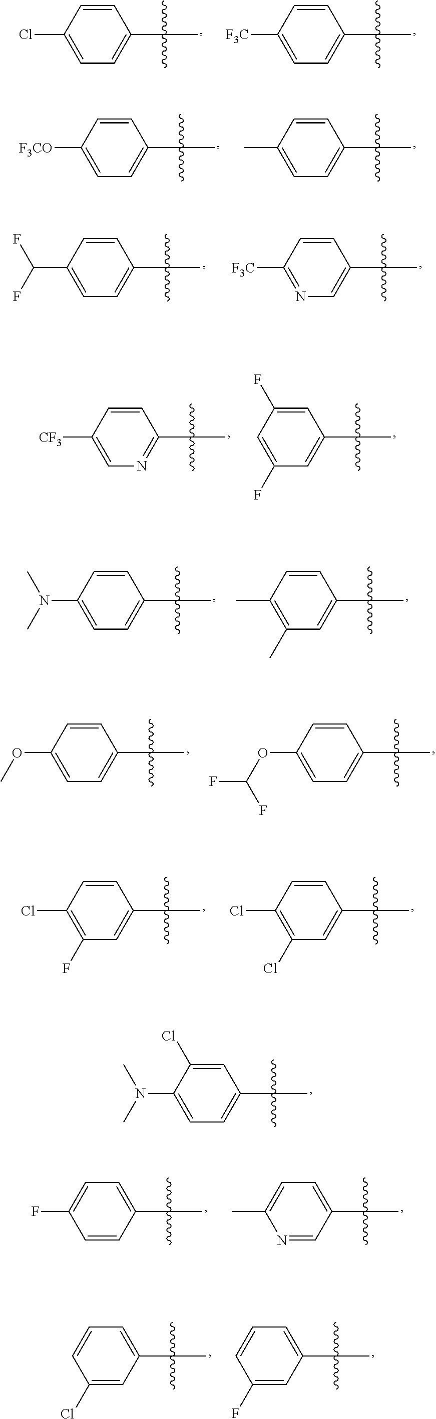

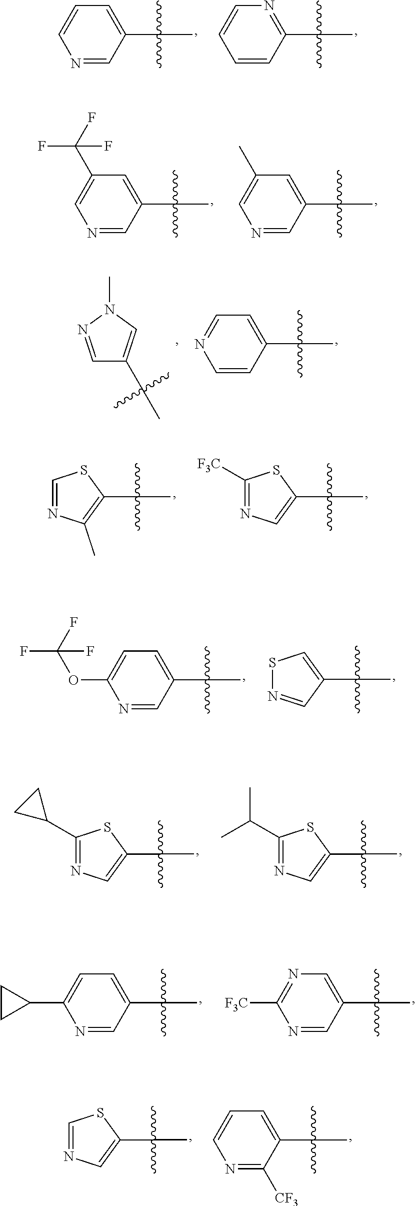

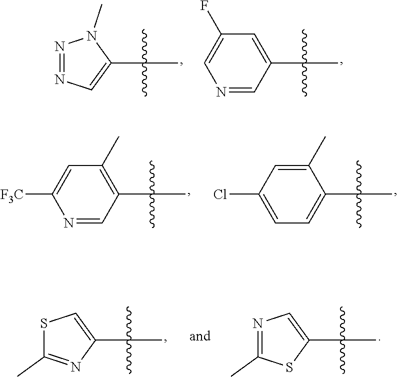

In some embodiments, ring A is chosen from

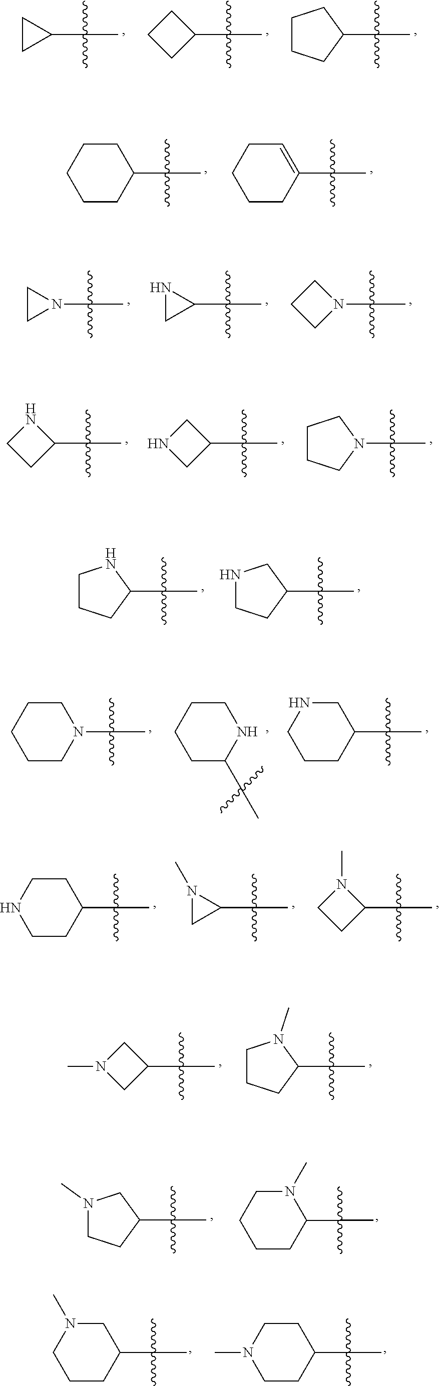

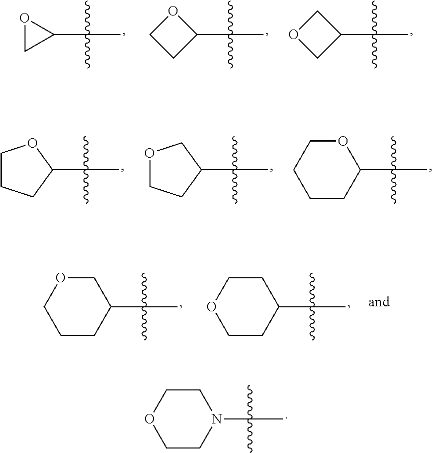

In some embodiments, ring A is chosen from

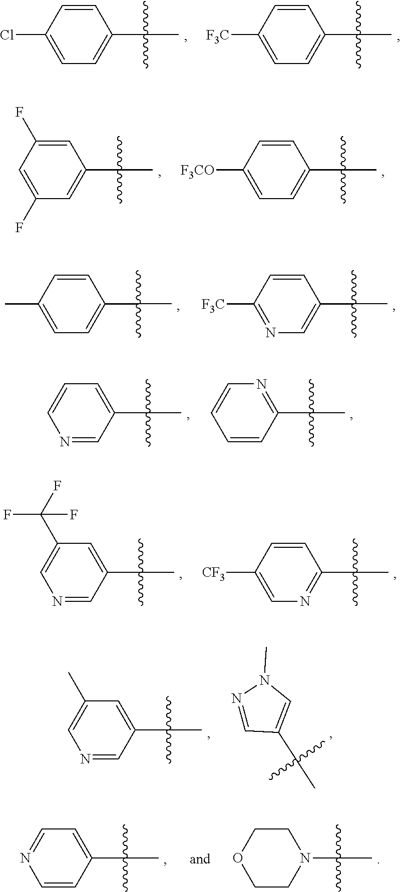

In some embodiments, ring B is chosen from

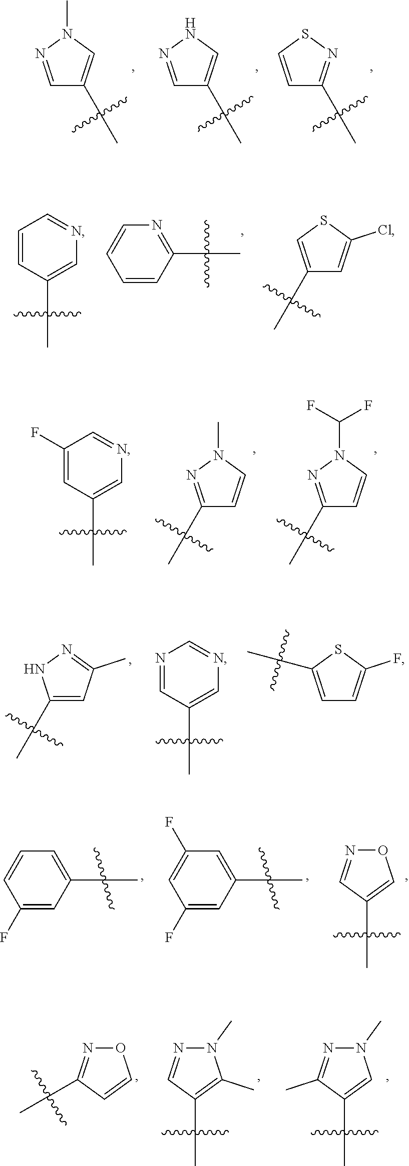

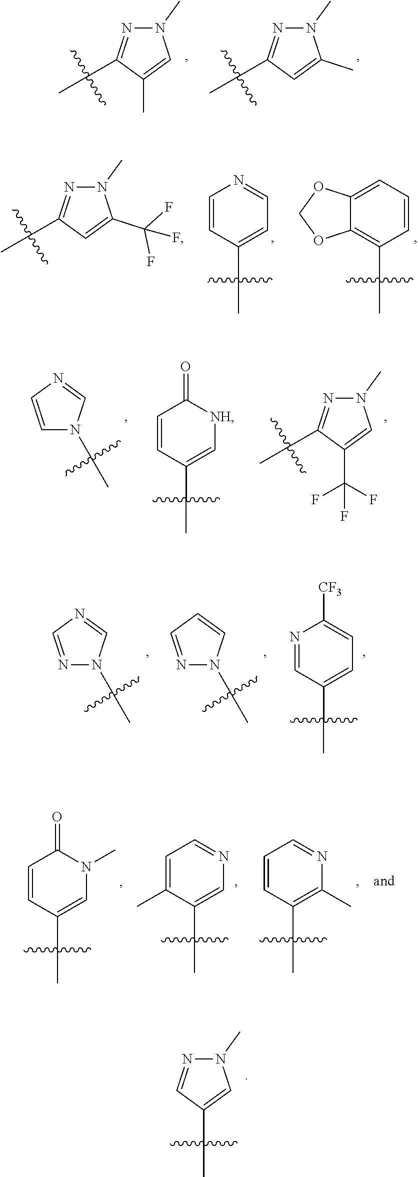

In some embodiments, ring B is from

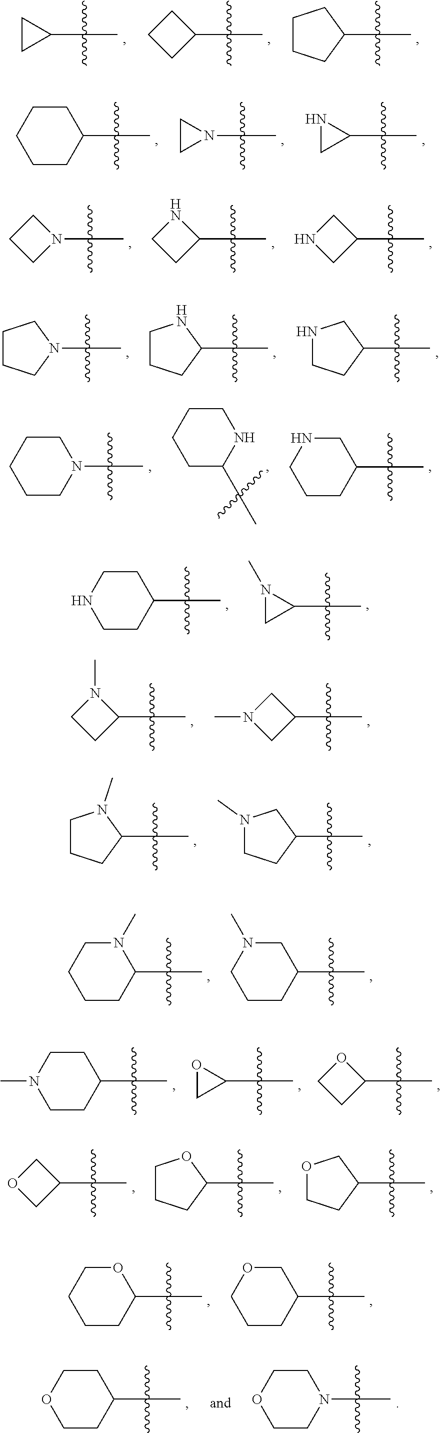

In some embodiments, ring B is chosen from

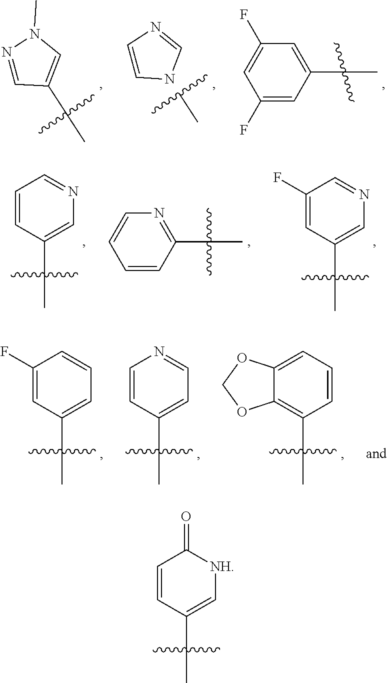

In some embodiments, R is chosen from methyl,

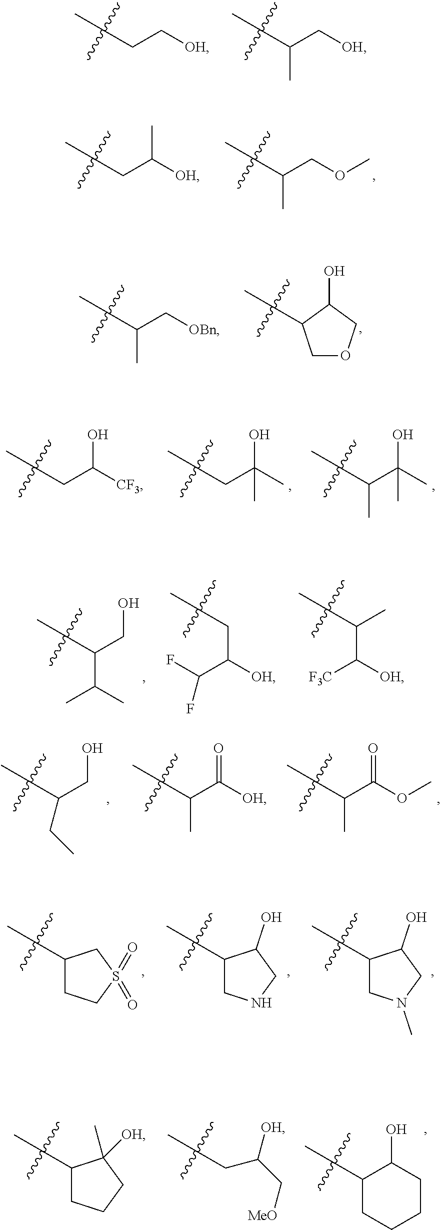

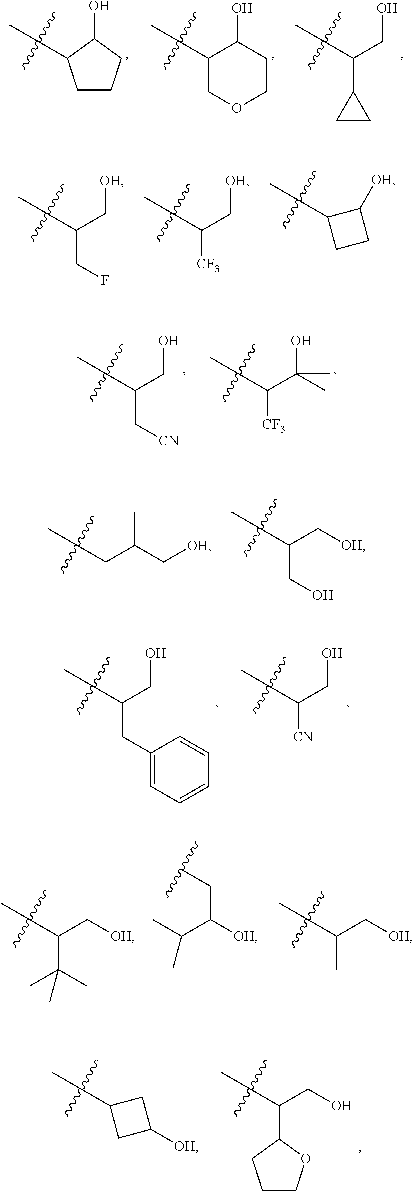

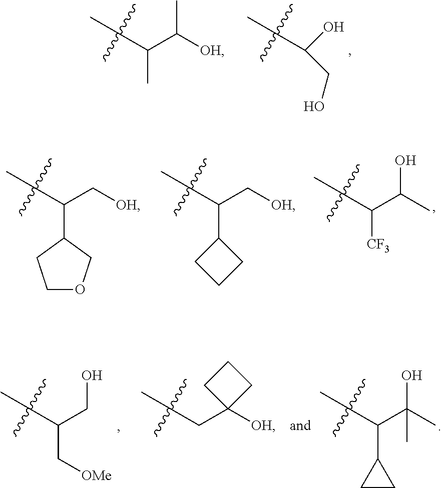

In some embodiments, R is chosen from methyl,

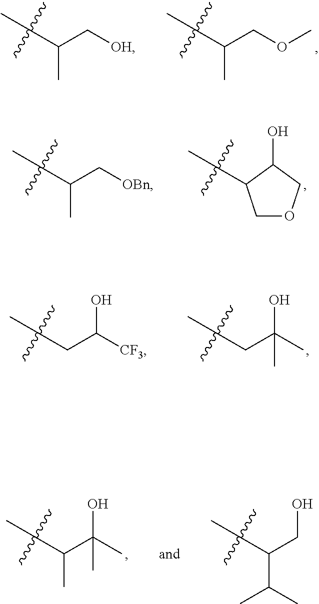

In some embodiments, an AhR antagonist according to the disclosure is a compound of Formula (Ib):

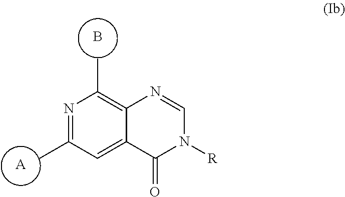

or a pharmaceutically acceptable salt thereof,
wherein:
ring A is chosen from optionally substituted heteroaryls and optionally substituted heterocycloalkyls;
ring B is chosen from optionally substituted heteroaryls and optionally substituted heterocycloalkyls; and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

In some embodiments, an AhR antagonist is any one of the compounds listed in Table 2.

TABLE 2

| Com- | |
|---|---|
| pound | |
| No. | Structure and name |
| 1 | 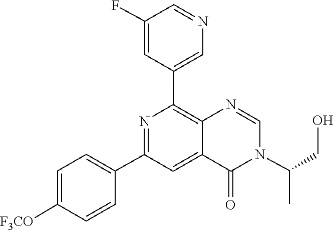 [SMILES: C[C@@H](CO)n1cnc2c(-c3cncc(F)c3)nc(-c3ccc(OC(F)(F)F)cc3)cc2c1=O] |
| | 8-(5-fluoropyridin-3-yl)-3-[(2S)- |
| | 1-hydroxypropan-2-yl]-6-[4- |
| | (trifluoromethoxy)phenyl]- |
| | 3H,4H-pyrido[3,4-d]pyrimidin- |
| | 4-one |
| 2 | 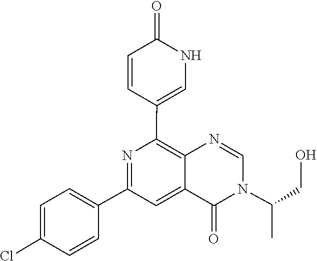 [SMILES: C[C@@H](CO)n1cnc2c(-c3ccc(=O)[nH]c3)nc(-c3ccc(Cl)cc3)cc2c1=O] |
| | 5-[6-(4-chlorophenyl)-3-[(2S)-1- |
| | hydroxypropan-2-yl]-4-oxo- |
| | 3H,4H-pyrido[3,4-d]pyrimidin- |
| | 8-yl]-1,2-dihydropyridin-2-one |
| 3 | 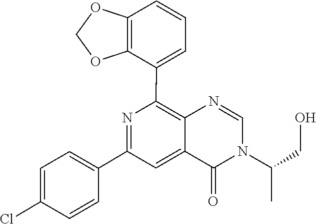 [SMILES: C[C@@H](CO)n1cnc2c(-c3cccc4c3OCO4)nc(-c3ccc(Cl)cc3)cc2c1=O] |
| | 8-(2H-1,3-benzodioxol-4-yl)-6- |
| | (4-chlorophenyl)-3-[(2S)-1- |
| | hydroxypropan-2-yl]-3H,4H- |
| | pyrido[3,4-d]pyrimidin-4-one |
| 4 | 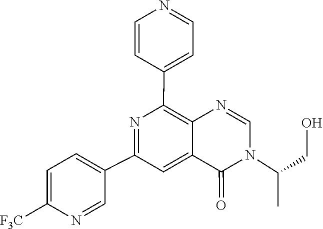 [SMILES: C[C@@H](CO)n1cnc2c(-c3ccncc3)nc(-c3ccc(C(F)(F)F)nc3)cc2c1=O] |
| | 3-[(2S)-1-hydroxypropan-2- |
| | yl]-8-(pyridin-4-yl)-6-[6- |
| | (trifluoromethyl)pyridin-3-yl]- |
| | 3H,4H-pyrido[3,4-d]pyrimidin- |
| | 4-one |
| 5 | 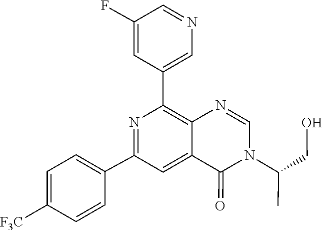 [SMILES: C[C@@H](CO)n1cnc2c(-c3cncc(F)c3)nc(-c3ccc(C(F)(F)F)cc3)cc2c1=O] |
| | 8-(5-fluoropyridin-3-yl)-3- |
| | [(2S)-1-hydroxypropan-2-yl]- |
| | 6-[4-(trifluoromethyl)phenyl]- |
| | 3H,4H-pyrido[3,4-d]pyrimidin- |
| | 4-one |
| 6 | 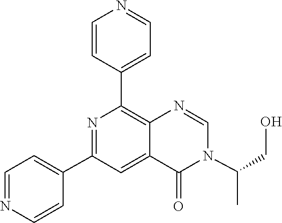 [SMILES: C[C@@H](CO)n1cnc2c(-c3ccncc3)nc(-c3ccncc3)cc2c1=O] |
| | 3-[(2S)-1-hydroxypropan-2- |
| | yl]-6,8-bis(pyridin-4-yl)- |
| | 3H,4H-pyrido[3,4-d]pyrimidin- |
| | 4-one |
| 7 | 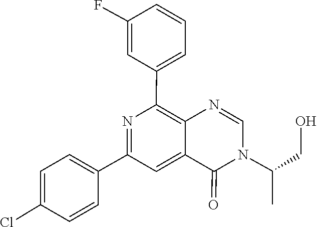 [SMILES: C[C@@H](CO)n1cnc2c(-c3cccc(F)c3)nc(-c3ccc(Cl)cc3)cc2c1=O] |
| | 6-(4-chlorophenyl)-8-(3- |
| | fluorophenyl)-3-[(2S)-1- |
| | hydroxypropan-2-yl]-3H,4H- |
| | pyrido[3,4-d]pyrimidin-4-one |
| 8 | 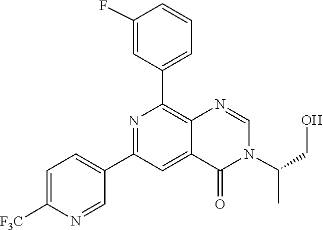 [SMILES: C[C@@H](CO)n1cnc2c(-c3cccc(F)c3)nc(-c3ccc(C(F)(F)F)nc3)cc2c1=O] |
| | 8-(3-fluorophenyl)-3-[(2S)-1- |
| | hydroxypropan-2-yl]-6-[6- |
| | (trifluoromethyl)pyridin-3-yl]- |
| | 3H,4H-pyrido[3,4-d]pyrimidin- |
| | 4-one |
| 9 | 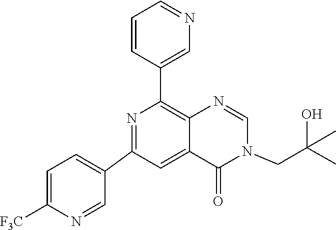 [SMILES: CC(C)(O)Cn1cnc2c(-c3cccnc3)nc(-c3ccc(C(F)(F)F)nc3)cc2c1=O] |
| | 3-(2-hydroxy-2-methylpropyl)- |
| | 8-(pyridin-3-yl)-6-(6- |
| | (trifluoromethyl)pyridin-3- |
| | yl)pyrido[3,4-d]pyrimidin- |
| | 4(3H)-one |
| 10 | 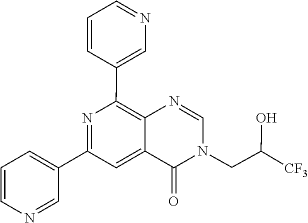 [SMILES: O=c1c2cc(-c3cccnc3)nc(-c3cccnc3)c2ncn1CC(O)C(F)(F)F] |
| | 6,8-di(pyridin-3-yl)-3-(3,3,3- |
| | trifluoro-2- |
| | hydroxypropyl)pyrido[3,4- |
| | d]pyrimidin-4(3H)-one |
| 11 | 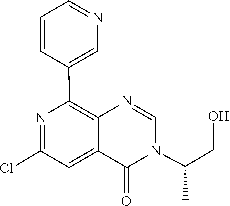 [SMILES: C[C@@H](CO)n1cnc2c(-c3cccnc3)nc(Cl)cc2c1=O] |
| | (S)-6-chloro-3-(1- |
| | hydroxypropan-2-yl)-8- |
| | (pyridin-3-yl)pyrido[3,4- |
| | d]pyrimidin-4(3H)-one |
| 12 | 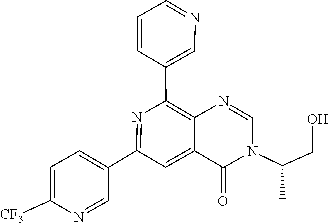 [SMILES: C[C@@H](CO)n1cnc2c(-c3cccnc3)nc(-c3ccc(C(F)(F)F)nc3)cc2c1=O] |
| | (S)-3-(1-hydroxypropan-2-yl)- |
| | 8-(pyridin-3-yl)-6-(6- |
| | (trifluoromethyl)pyridin-3- |
| | yl)pyrido[3,4-d]pyrimidin- |
| | 4(3H)-one |
| 13 | 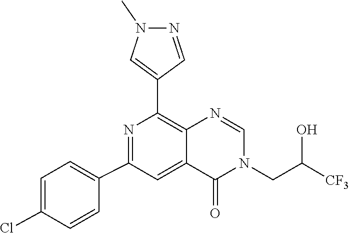 [SMILES: Cn1cc(-c2nc(-c3ccc(Cl)cc3)cc3c(=O)n(CC(O)C(F)(F)F)cnc23)cn1] |
| | 6-(4-chlorophenyl)-8-(pyridin- |
| | 3-yl)-3-(3,3,3-trifluoro-2- |
| | hydroxypropyl)pyrido[3,4- |
| | d]pyrimidin-4(3H)-one |
| 14 | 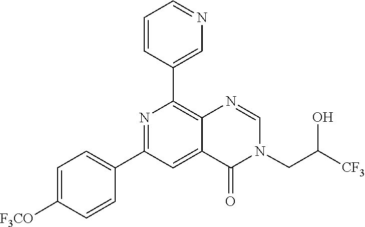 [SMILES: O=c1c2cc(-c3ccc(OC(F)(F)F)cc3)nc(-c3cccnc3)c2ncn1CC(O)C(F)(F)F] |
| | 8-(pyridin-3-yl)-3-(3,3,3- |
| | trifluoro-2-hydroxypropyl)-6- |
| | (4- |
| | (trifluoromethoxy)phenyl)pyrido |
| | [3,4-d]pyrimidin-4(3H)-one |
| 15 | 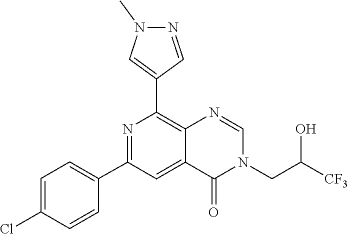 [SMILES: Cn1cc(-c2nc(-c3ccc(Cl)cc3)cc3c(=O)n(CC(O)C(F)(F)F)cnc23)cn1] |
| | 6-(4-chlorophenyl)-8-(1- |
| | methyl-1H-pyrazol-4-yl)-3- |
| | (3,3,3-trifluoro-2- |
| | hydroxypropyl)pyrido[3,4- |
| | d]pyrimidin-4(3H)-one |
| 16 | 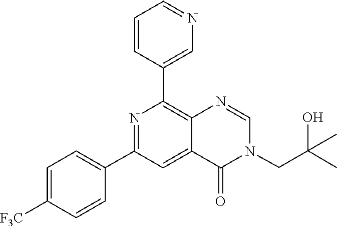 [SMILES: CC(C)(O)Cn1cnc2c(-c3cccnc3)nc(-c3ccc(C(F)(F)F)cc3)cc2c1=O] |
| | 3-(2-hydroxy-2-methylpropyl)- |
| | 8-(pyridin-3-yl)-6-(4- |
| | (trifluoromethyl)phenyl)pyrido |
| | [3,4-d]pyrimidin-4(3H)-one |
| 17 | 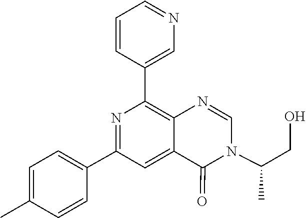 [SMILES: Cc1ccc(-c2cc3c(=O)n([C@@H](C)CO)cnc3c(-c3cccnc3)n2)cc1] |
| | (S)-3-(1-hydroxypropan-2-yl)- |
| | 8-(pyridin-3-yl)-6-(p-tolyl) |
| | pyrido[3,4-d]pyrimidin- |
| | 4(3H)-one |
| 18 | 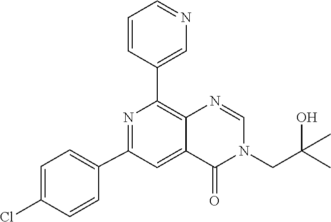 [SMILES: CC(C)(O)Cn1cnc2c(-c3cccnc3)nc(-c3ccc(Cl)cc3)cc2c1=O] |
| | 6-(4-chlorophenyl)-3-(2- |
| | hydroxy-2-methylpropyl)-8- |
| | (pyridin-3-yl)pyrido[3,4- |
| | d]pyrimidin-4(3H)-one |
| 19 | 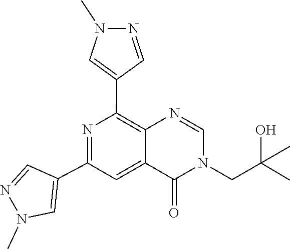 [SMILES: Cn1cc(-c2cc3c(=O)n(CC(C)(C)O)cnc3c(-c3cnn(C)c3)n2)cn1] |
| | 3-(2-hydroxy-2-methylpropyl)- |
| | 6,8-bis(1-methyl-1H-pyrazol- |
| | 4-yl)pyrido[3,4-d]pyrimidin- |
| | 4(3H)-one |
| 20 | 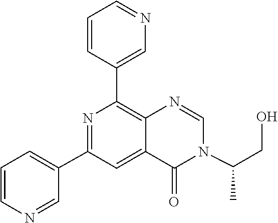 [SMILES: C[C@@H](CO)n1cnc2c(-c3cccnc3)nc(-c3cccnc3)cc2c1=O] |
| | (S)-3-(1-hydroxypropan-2-yl)- |
| | 6,8-di(pyridin-3-yl)pyrido[3,4- |
| | d]pyrimidin-4(3H)-one |
| 21 | 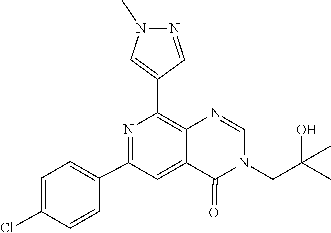 [SMILES: Cn1cc(-c2nc(-c3ccc(Cl)cc3)cc3c(=O)n(CC(C)(C)O)cnc23)cn1] |
| | 6-(4-chlorophenyl)-3-(2- |
| | hydroxy-2-methylpropyl)-8-(1- |
| | methyl-1H-pyrazol-4- |
| | yl)pyrido[3,4-d]pyrimidin- |
| | 4(3H)-one |
| 22 | 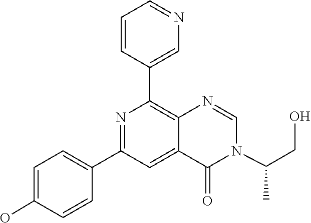 |
| | (S)-3-(1-hydroxypropan-2-yl)- |
| | 8-(pyridin-3-yl)-6-(4- |
| | (trifluoromethoxy)phenyl)pyrido |
| | [3,4-d]pyrimidin-4(3H)-one |
| 23 | 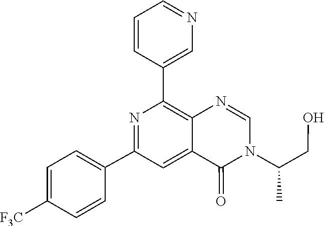 [SMILES: C[C@@H](CO)n1cnc2c(-c3cccnc3)nc(-c3ccc(C(F)(F)F)cc3)cc2c1=O] |
| | (S)-3-(1-hydroxypropan-2-yl)- |
| | 8-(pyridin-3-yl)-6-(4- |
| | (trifluoromethyl)phenyl)pyrido |
| | [3,4-d]pyrimidin-4(3H)-one |
| 24 | 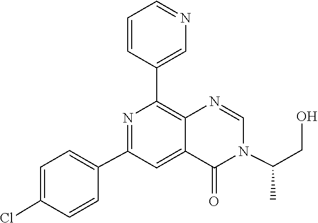 [SMILES: C[C@@H](CO)n1cnc2c(-c3cccnc3)nc(-c3ccc(Cl)cc3)cc2c1=O] |
| | (S)-6-(4-chlorophenyl)-3-(1- |
| | hydroxypropan-2-yl)-8- |
| | (pyridin-3-yl)pyrido[3,4- |
| | d]pyrimidin-4(3H)-one |
| 25 | 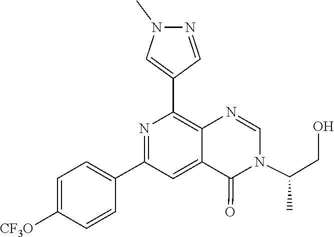 |
| | (S)-3-(1-hydroxypropan-2-yl)- |
| | 8-(1-methyl-1H-pyrazol-4-yl)- |
| | 6-(4- |
| | (trifluoromethoxy)phenyl)pyrido |
| | [3,4-d]pyrimidin-4(3H)-one |
| 26 | 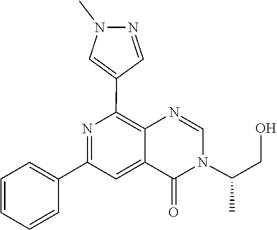 [SMILES: C[C@@H](CO)n1cnc2c(-c3cnn(C)c3)nc(-c3ccccc3)cc2c1=O] |
| | (S)-3-(1-hydroxypropan-2-yl)-8- |
| | (1-methyl-1H-pyrazol-4-yl)-6- |
| | phenylpyrido[3,4-d]pyrimidin- |
| | 4(3H)-one |
| 27 | 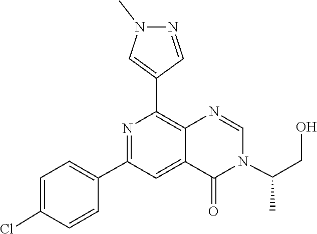 [SMILES: C[C@@H](CO)n1cnc2c(-c3cnn(C)c3)nc(-c3ccc(Cl)cc3)cc2c1=O] |
| | (S)-6-(4-chlorophenyl)-3-(1- |
| | hydroxypropan-2-yl)-8-(1- |
| | methyl-1H-pyrazol-4- |
| | yl)pyrido[3,4-d]pyrimidin- |
| | 4(3H)-one |
| 28 | 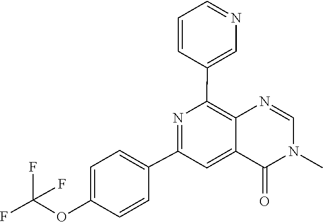 [SMILES: Cn1cnc2c(-c3cccnc3)nc(-c3ccc(OC(F)(F)F)cc3)cc2c1=O] |
| | 3-methyl-8-(pyridin-3-yl)-6-(4- |
| | (trifluoromethoxy)phenyl)pyrido |
| | [3,4-d]pyrimidin-4(3H)-one |
| 29 | 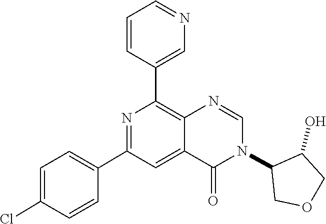 [SMILES: O=c1c2cc(-c3ccc(Cl)cc3)nc(-c3cccnc3)c2ncn1[C@@H]1COC[C@H]1O] |
| | rac-6-(4-chlorophenyl)-3- |
| | ((trans)-4- |
| | hydroxytetrahydrofuran-3-yl)- |
| | 8-(pyridin-3-yl)pyrido[3,4- |
| | d]pyrimidin-4(3H)-one |
| 30 | 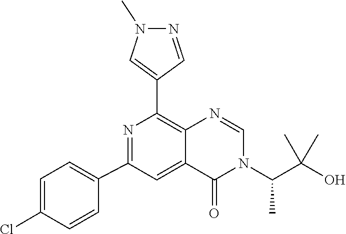 [SMILES: C[C@H](n1cnc2c(-c3cnn(C)c3)nc(-c3ccc(Cl)cc3)cc2c1=O)C(C)(C)O] |
| | (S)-6-(4-chlorophenyl)-3-(3- |
| | hydroxy-3-methylbutan-2-yl)-8- |
| | (1-methyl-1H-pyrazol-4- |
| | yl)pyrido[3,4-d]pyrimidin- |
| | 4(3H)-one |
| 31 | 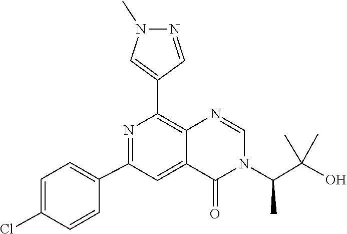 [SMILES: C[C@@H](n1cnc2c(-c3cnn(C)c3)nc(-c3ccc(Cl)cc3)cc2c1=O)C(C)(C)O] |
| | (R)-6-(4-chlorophenyl)-3-(3- |
| | hydroxy-3-methylbutan-2-yl)- |
| | 8-(1-methyl-1H-pyrazol-4- |
| | yl)pyrido[3,4-d]pyrimidin- |
| | 4(3H)-one |
| 32 | 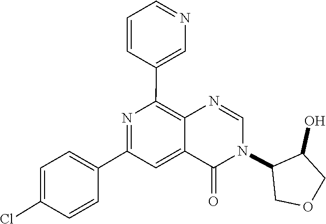 [SMILES: O=c1c2cc(-c3ccc(Cl)cc3)nc(-c3cccnc3)c2ncn1[C@@H]1COC[C@@H]1O] |
| | rac-6-(4-chlorophenyl)-3-((cis)- |
| | 4-hydroxytetrahydrofuran-3-yl)- |
| | 8-(pyridin-3-yl)pyrido[3,4- |
| | d]pyrimidin-4(3H)-one |
| 33 | 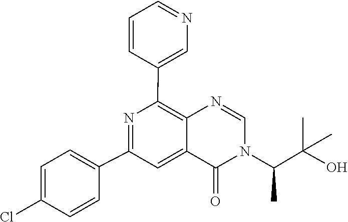 [SMILES: C[C@@H](n1cnc2c(-c3cccnc3)nc(-c3ccc(Cl)cc3)cc2c1=O)C(C)(C)O] |
| | (R)-6-(4-chlorophenyl)-3-(3- |
| | hydroxy-3-methylbutan-2-yl)- |
| | 8-(pyridin-3-yl)pyrido[3,4- |
| | d]pyrimidin-4(3H)-one |
| 34 | 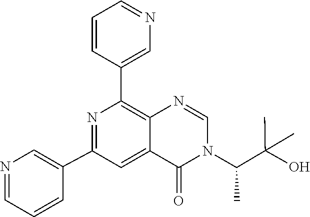 [SMILES: C[C@H](n1cnc2c(-c3cccnc3)nc(-c3cccnc3)cc2c1=O)C(C)(C)O] |
| | (S)-3-(3-hydroxy-3- |
| | methylbutan-2-yl)-6,8- |
| | di(pyridin-3-yl)pyrido[3,4- |
| | d]pyrimidin-4(3H)-one |
| 35 | 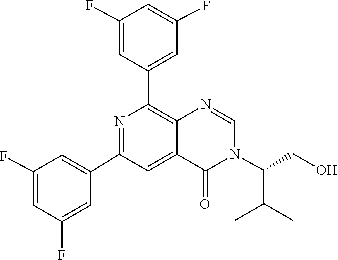 [SMILES: CC(C)[C@@H](CO)n1cnc2c(-c3cc(F)cc(F)c3)nc(-c3cc(F)cc(F)c3)cc2c1=O] |
| | (S)-6,8-bis(3,5- |
| | difluorophenyl)-3-(1-hydroxy- |
| | 3-methylbutan-2-yl)pyrido[3,4- |
| | d]pyrimidin-4(3H)-one |
| 36 | 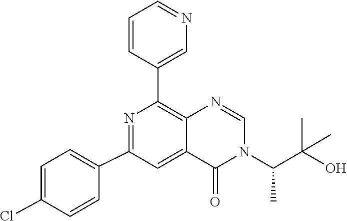 [SMILES: C[C@H](n1cnc2c(-c3cccnc3)nc(-c3ccc(Cl)cc3)cc2c1=O)C(C)(C)O] |
| | (S)-6-(4-chlorophenyl)-3-(3- |
| | hydroxy-3-methylbutan-2-yl)-8- |
| | (pyridin-3-yl)pyrido[3,4- |
| | d]pyrimidin-4(3H)-one |
| 37 | 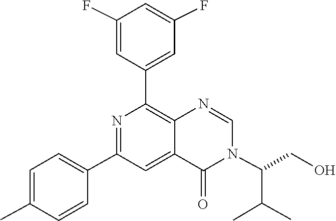 [SMILES: Cc1ccc(-c2cc3c(=O)n([C@H](CO)C(C)C)cnc3c(-c3cc(F)cc(F)c3)n2)cc1] |
| | (S)-8-(3,5-difluorophenyl)-3- |
| | (1-hydroxy-3-methylbutan-2- |
| | yl)-6-(p-tolyl)pyrido[3,4- |
| | d]pyrimidin-4(3H)-one |
| 38 | 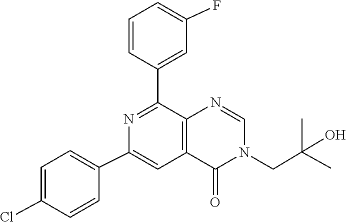 [SMILES: CC(C)(O)Cn1cnc2c(-c3cccc(F)c3)nc(-c3ccc(Cl)cc3)cc2c1=O] |
| | 6-(4-chlorophenyl)-8-(3- |
| | fluorophenyl)-3-(2-hydroxy-2- |
| | methylpropyl)pyrido[3,4- |
| | d]pyrimidin-4(3H)-one |
| 39 | 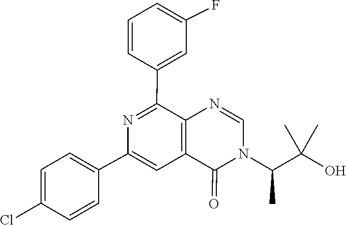 [SMILES: C[C@@H](n1cnc2c(-c3cccc(F)c3)nc(-c3ccc(Cl)cc3)cc2c1=O)C(C)(C)O] |
| | (R)-6-(4-chlorophenyl)-8-(3- |
| | fluorophenyl)-3-(3-hydroxy-3- |
| | methylbutan-2-yl)pyrido[3,4- |
| | d]pyrimidin-4(3H)-one |
| 40 | 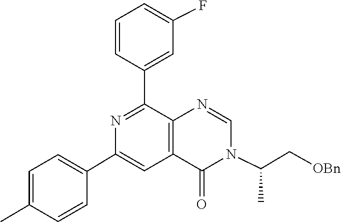 [SMILES: Cc1ccc(-c2cc3c(=O)n([C@@H](C)COCc4ccccc4)cnc3c(-c3cccc(F)c3)n2)cc1] |
| | (S)-3-(1-(benzyloxy)propan-2- |
| | yl)-8-(3-fluorophenyl)-6-(p- |
| | tolyl)pyrido[3,4-d]pyrimidin- |
| | 4(3H)-one |
| 41 | 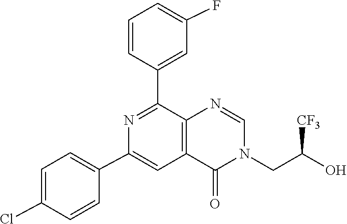 [SMILES: O=c1c2cc(-c3ccc(Cl)cc3)nc(-c3cccc(F)c3)c2ncn1C[C@H](O)C(F)(F)F] |
| | 6-(4-chlorophenyl)-8-(3- |
| | fluorophenyl)-3-(3,3,3- |
| | trifluoro-2- |
| | hydroxypropyl)pyrido[3,4- |
| | d]pyrimidin-4(3H)-one, |
| | enantiomer 1 |
| 42 | 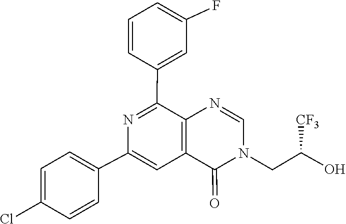 [SMILES: O=c1c2cc(-c3ccc(Cl)cc3)nc(-c3cccc(F)c3)c2ncn1C[C@@H](O)C(F)(F)F] |
| | 6-(4-chlorophenyl)-8-(3- |
| | fluorophenyl)-3-(3,3,3-trifluoro- |
| | 2-hydroxypropyl)pyrido[3,4- |
| | d]pyrimidin-4(3H)-one, |
| | enantiomer 2 |
| 43 | 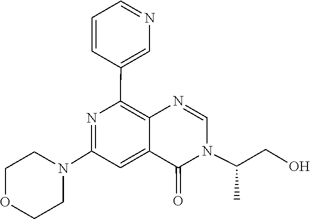 [SMILES: C[C@@H](CO)n1cnc2c(-c3cccnc3)nc(N3CCOCC3)cc2c1=O] |
| | (S)-3-(1-hydroxypropan-2-yl)- |
| | 6-morpholino-8-(pyridin-3- |
| | yl)pyrido[3,4-d]pyrimidin- |
| | 4(3H)-one |
| 44 | 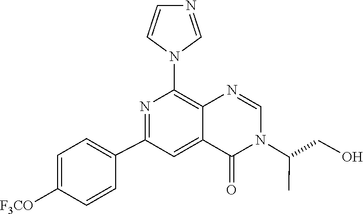 [SMILES: C[C@@H](CO)n1cnc2c(-n3ccnc3)nc(-c3ccc(OC(F)(F)F)cc3)cc2c1=O] |
| | (S)-3-(1-hydroxypropan-2-yl)-8- |
| | (1H-imidazol-1-yl)-6-(4- |
| | (trifluoromethoxy)phenyl)pyrido |
| | [3,4-d]pyrimidin-4(3H)-one |
| 45 | 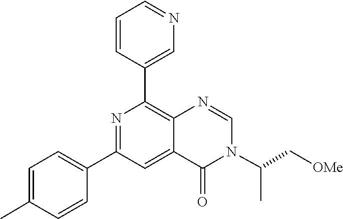 [SMILES: COC[C@H](C)n1cnc2c(-c3cccnc3)nc(-c3ccc(C)cc3)cc2c1=O] |
| | (S)-3-(1-methoxypropan-2-yl)- |
| | 8-(pyridin-3-yl)-6-(p- |
| | tolyl)pyrido[3,4-d]pyrimidin- |
| | 4(3H)-one |
| 46 | 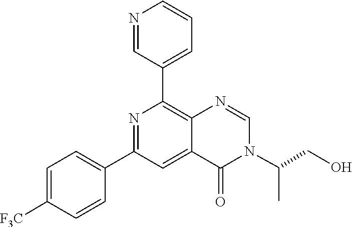 [SMILES: C[C@@H](CO)n1cnc2c(-c3cccnc3)nc(-c3ccc(C(F)(F)F)cc3)cc2c1=O] |
| | (S)-3-(1-hydroxypropan-2-yl)-8- |
| | (pyridin-3-yl)-6-(5- |
| | (trifluoromethyl)pyridin-2- |
| | yl)pyrido[3,4-d]pyrimidin- |
| | 4(3H)-one |
| 47 | 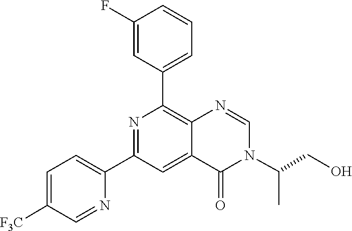 [SMILES: C[C@@H](CO)n1cnc2c(-c3cccc(F)c3)nc(-c3ccc(C(F)(F)F)cn3)cc2c1=O] |
| | (S)-8-(3-fluorophenyl)-3-(1- |
| | hydroxypropan-2-yl)-6-(5- |
| | (trifluoromethyl)pyridin-2- |
| | yl)pyrido[3,4-d]pyrimidin- |
| | 4(3H)-one |
| 48 | 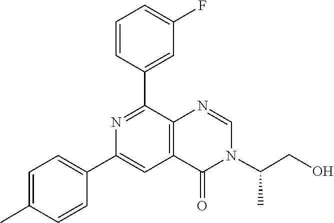 [SMILES: Cc1ccc(-c2cc3c(=O)n([C@@H](C)CO)cnc3c(-c3cccc(F)c3)n2)cc1] |
| | (S)-8-(3-fluorophenyl)-3-(1- |
| | hydroxypropan-2-yl)-6-(p- |
| | tolyl)pyrido[3,4-d]pyrimidin- |
| | 4(3H)-one |
| 49 | 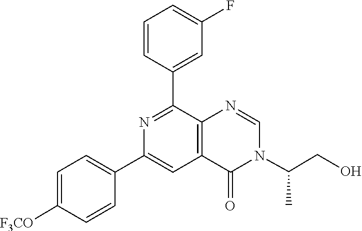 [SMILES: C[C@@H](CO)n1cnc2c(-c3cccc(F)c3)nc(-c3ccc(OC(F)(F)F)cc3)cc2c1=O] |
| | (S)-8-(3-fluorophenyl)-3-(1- |
| | hydroxypropan-2-yl)-6-(4- |
| | (trifluoromethoxy)phenyl)pyrido |
| | [3,4-d]pyrimidin-4(3H)-one |
| 50 | 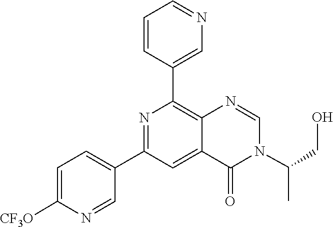 |
| | (S)-3-(1-hydroxypropan-2-yl)-8- |
| | (pyridin-3-yl)-6-(6- |
| | (trifluoromethoxy)pyridin-3- |
| | yl)pyrido[3,4-d]pyrimidin- |
| | 4(3H)-one |
| 51 | 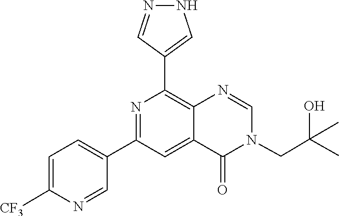 [SMILES: CC(C)(O)Cn1cnc2c(-c3cn[nH]c3)nc(-c3ccc(C(F)(F)F)nc3)cc2c1=O] |
| | (S)-3-(2-hydroxy-2- |
| | methylpropyl)-8-(1H-pyrazol- |
| | 4-yl)-6-(6- |
| | (trifluoromethyl)pyridin-3- |
| | yl)pyrido[3,4-d]pyrimidin- |
| | 4(3H)-one |
| 52 | 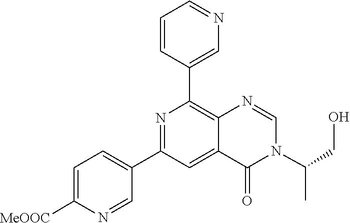 [SMILES: COC(=O)c1ccc(-c2cc3c(=O)n([C@@H](C)CO)cnc3c(-c3cccnc3)n2)cn1] |
| | methyl (S)-5-(3-(1- |
| | hydroxypropan-2-yl)-4-oxo-8- |
| | (pyridin-3-yl)-3,4- |
| | dihydropyrido[3,4-d]pyrimidin- |
| | 6-yl)picolinate |
| 53 | 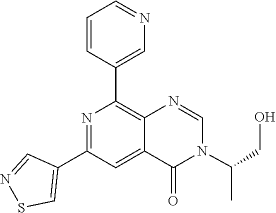 [SMILES: C[C@@H](CO)n1cnc2c(-c3cccnc3)nc(-c3cnsc3)cc2c1=O] |
| | (S)-3-(1-hydroxypropan-2-yl)- |
| | 6-(isothiazol-4-yl)-8-(pyridin- |
| | 3-yl)pyrido[3,4-d]pyrimidin- |
| | 4(3H)-one |
| 54 | 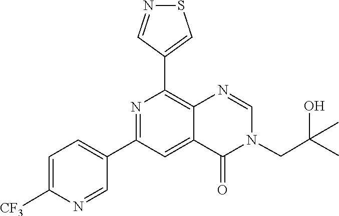 [SMILES: CC(C)(O)Cn1cnc2c(-c3cnsc3)nc(-c3ccc(C(F)(F)F)nc3)cc2c1=O] |
| | 3-(2-hydroxy-2-methylpropyl)- |
| | 8-(isothiazol-4-yl)-6-(6- |
| | (trifluoromethyl)pyridin-3- |
| | yl)pyrido[3,4-d]pyrimidin- |
| | 4(3H)-one |
| 55 | 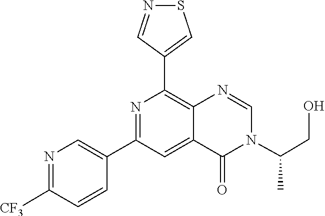 [SMILES: C[C@@H](CO)n1cnc2c(-c3cnsc3)nc(-c3ccc(C(F)(F)F)nc3)cc2c1=O] |
| | (S)-3-(1-hydroxypropan-2-yl)- |
| | 8-(isothiazol-4-yl)-6-(6- |
| | (trifluoromethyl)pyridin-3- |
| | yl)pyrido[3,4-d]pyrimidin- |
| | 4(3H)-one |
| 56 | 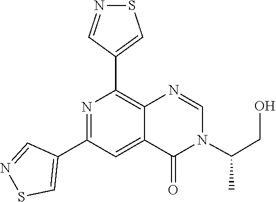 [SMILES: C[C@@H](CO)n1cnc2c(-c3cnsc3)nc(-c3cnsc3)cc2c1=O] |
| | (S)-3-(1-hydroxypropan-2-yl)- |
| | 6,8-di(isothiazol-4- |
| | yl)pyrido[3,4-d]pyrimidin- |
| | 4(3H)-one |
| 57 | 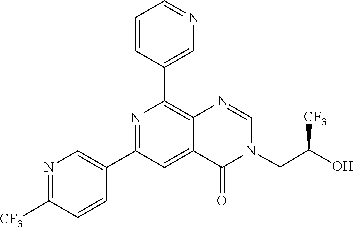 [SMILES: O=c1c2cc(-c3ccc(C(F)(F)F)nc3)nc(-c3cccnc3)c2ncn1C[C@H](O)C(F)(F)F] |
| | (S)-8-(pyridin-3-yl)-3-(3,3,3- |
| | trifluoro-2-hydroxypropyl)-6- |
| | (6-(trifluoromethyl)pyridin-3- |
| | yl)pyrido[3,4-d]pyrimidin- |
| | 4(3H)-one |
| 58 | 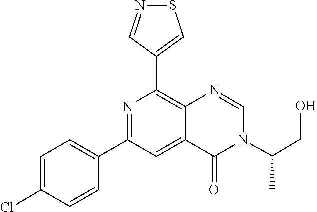 [SMILES: C[C@@H](CO)n1cnc2c(-c3cnsc3)nc(-c3ccc(Cl)cc3)cc2c1=O] |
| | (S)-6-(4-chlorophenyl)-3-(1- |
| | hydroxypropan-2-yl)-8- |
| | (isothiazol-4-yl)pyrido[3,4- |
| | d]pyrimidin-4(3H)-one |
| 59 | 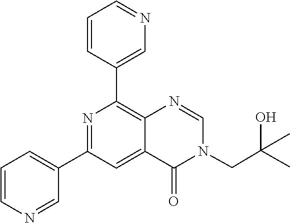 [SMILES: CC(C)(O)Cn1cnc2c(-c3cccnc3)nc(-c3cccnc3)cc2c1=O] |
| | 3-(2-hydroxy-2-methylpropyl)- |
| | 6,8-di(pyridin-3-yl)pyrido[3,4- |
| | d]pyrimidin-4(3H)-one |
| 60 | 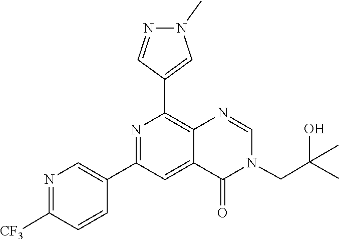 [SMILES: Cn1cc(-c2nc(-c3ccc(C(F)(F)F)nc3)cc3c(=O)n(CC(C)(C)O)cnc23)cn1] |
| | 3-(2-hydroxy-2-methylpropyl)- |
| | 8-(1-methyl-1H-pyrazol-4-yl)-6- |
| | (6-(trifluoromethyl)pyridin-3- |
| | yl)pyrido[3,4-d]pyrimidin- |
| | 4(3H)-one |
| 61 | 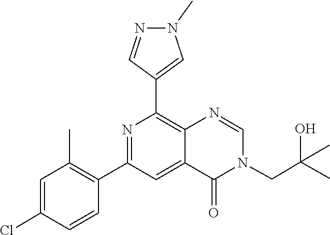 [SMILES: Cc1cc(Cl)ccc1-c1cc2c(=O)n(CC(C)(C)O)cnc2c(-c2cnn(C)c2)n1] |
| | 6-(4-chloro-2-methylphenyl)-3- |
| | (2-hydroxy-2-methylpropyl)-8- |
| | (1-methyl-1H-pyrazol-4- |
| | yl)pyrido[3,4-d]pyrimidin- |
| | 4(3H)-one |
| 62 | 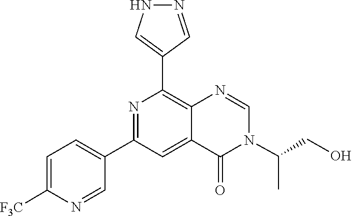 [SMILES: C[C@@H](CO)n1cnc2c(-c3cn[nH]c3)nc(-c3ccc(C(F)(F)F)nc3)cc2c1=O] |
| | (S)-3-(1-hydroxypropan-2-yl)-8- |
| | (1H-pyrazol-4-yl)-6-(6- |
| | (trifluoromethyl)pyridin-3- |
| | yl)pyrido[3,4-d]pyrimidin- |
| | 4(3H)-one |
| 63 | 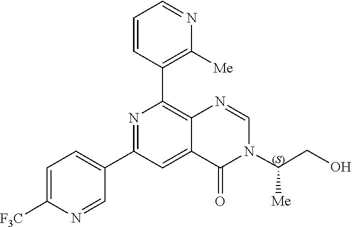 |
| | (S)-3-(1-hydroxypropan-2-yl)-8- |
| | (2-methylpyridin-3-yl)-6-(6- |
| | (trifluoromethyl)pyridin-3- |
| | yl)pyrido[3,4-d]pyrimidin- |
| | 4(3H)-one |
| 64 | 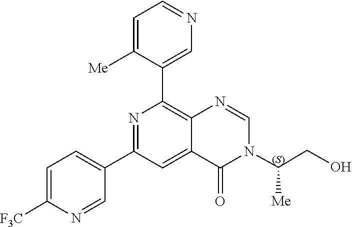 |
| | (S)-3-(1-hydroxypropan-2-yl)-8- |
| | (4-methylpyridin-3-yl)-6-(6- |
| | (trifluoromethyl)pyridin-3- |
| | yl)pyrido[3,4-d]pyrimidin- |
| | 4(3H)-one |
| 65 | 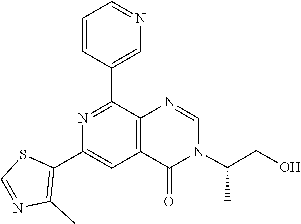 [SMILES: Cc1ncsc1-c1cc2c(=O)n([C@@H](C)CO)cnc2c(-c2cccnc2)n1] |
| | (S)-3-(1-hydroxypropan-2-yl)- |
| | 6-(4-methylthiazol-5-yl)-8- |
| | (pyridin-3-yl)pyrido[3,4- |
| | d]pyrimidin-4(3H)-one |
| 66 | 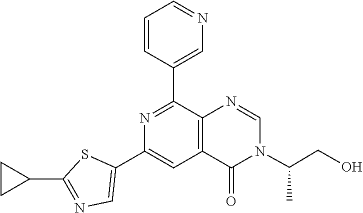 [SMILES: C[C@@H](CO)n1cnc2c(-c3cccnc3)nc(-c3cnc(C4CC4)s3)cc2c1=O] |
| | (S)-6-(2-cyclopropylthiazol-5- |
| | yl)-3-(1-hydroxypropan-2-yl)-8- |
| | (pyridin-3-yl)pyrido[3,4- |
| | d]pyrimidin-4(3H)-one |
| 67 | 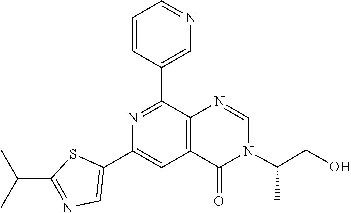 [SMILES: CC(C)c1ncc(-c2cc3c(=O)n([C@@H](C)CO)cnc3c(-c3cccnc3)n2)s1] |
| | (S)-3-(1-hydroxypropan-2-yl)- |
| | 6-(2-isopropylthiazol-5-yl)-8- |
| | (pyridin-3-yl)pyrido[3,4- |
| | d]pyrimidin-4(3H)-one |
| 68 | 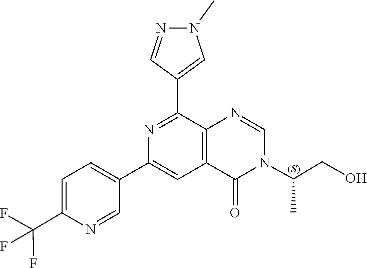 |
| | (S)-3-(1-hydroxypropan-2-yl)-8- |
| | (1-methyl-1H-pyrazol-4-yl)-6- |
| | (6-(trifluoromethyl)pyridin-3- |
| | yl)pyrido[3,4-d]pyrimidin- |
| | 4(3H)-one |
| 69 | 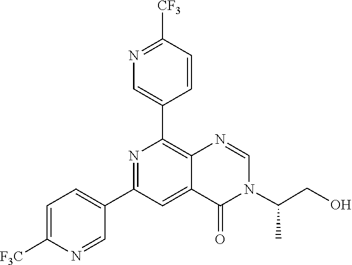 [SMILES: C[C@@H](CO)n1cnc2c(-c3ccc(C(F)(F)F)nc3)nc(-c3ccc(C(F)(F)F)nc3)cc2c1=O] |
| | (S)-3-(1-hydroxypropan-2-yl)- |
| | 6,8-bis(6- |
| | (trifluoromethyl)pyridin-3- |
| | yl)pyrido[3,4-d]pyrimidin- |
| | 4(3H)-one |
| 70 | 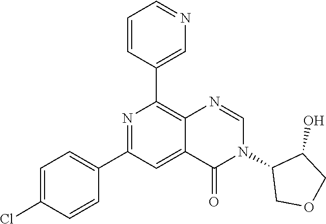 [SMILES: O=c1c2cc(-c3ccc(Cl)cc3)nc(-c3cccnc3)c2ncn1[C@H]1COC[C@H]1O] |
| | 6-(4-chlorophenyl)-3-((3S,4S)-4- |
| | hydroxytetrahydrofuran-3-yl)-8- |
| | (pyridin-3-yl)pyrido[3,4- |
| | d]pyrimidin-4(3H)-one. |
| 71 | 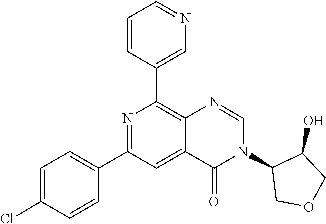 [SMILES: O=c1c2cc(-c3ccc(Cl)cc3)nc(-c3cccnc3)c2ncn1[C@@H]1COC[C@@H]1O] |
| | 6-(4-chlorophenyl)-3-((3R,4R)- |
| | 4-hydroxytetrahydrofuran-3-yl)- |
| | 8-(pyridin-3-yl)pyrido[3,4- |
| | d]pyrimidin-4(3H)-one |
| 72 | 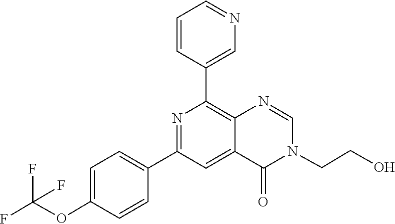 [SMILES: O=c1c2cc(-c3ccc(OC(F)(F)F)cc3)nc(-c3cccnc3)c2ncn1CCO] |
| | 3-(2-hydroxyethyl)-8-(pyridin-3- |
| | yl)-6-(4- |
| | (trifluoromethoxy)phenyl)pyrido |
| | [3,4-d]pyrimidin-4(3H)-one |
| 73 | 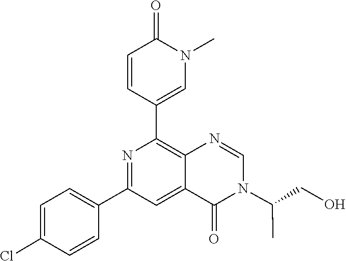 [SMILES: C[C@@H](CO)n1cnc2c(-c3ccc(=O)n(C)c3)nc(-c3ccc(Cl)cc3)cc2c1=O] |
| | (S)-6-(4-chlorophenyl)-3-(1- |
| | hydroxypropan-2-yl)-8-(1- |
| | methyl-6-oxo-1,6- |
| | dihydropyridin-3-yl)pyrido[3,4- |
| | d]pyrimidin-4(3H)-one |
| 74 | 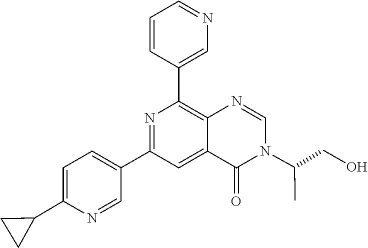 [SMILES: C[C@@H](CO)n1cnc2c(-c3cccnc3)nc(-c3ccc(C4CC4)nc3)cc2c1=O] |
| | (S)-6-(6-cyclopropylpyridin-3- |
| | yl)-3-(1-hydroxypropan-2-yl)-8- |
| | (pyridin-3-yl)pyrido[3,4- |
| | d]pyrimidin-4(3H)-one |
| 75 | 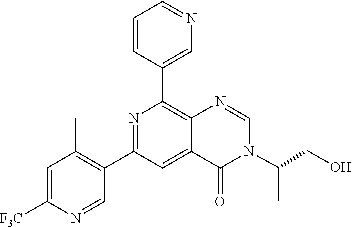 [SMILES: Cc1cc(C(F)(F)F)ncc1-c1cc2c(=O)n([C@@H](C)CO)cnc2c(-c2cccnc2)n1] |
| | (S)-3-(1-hydroxypropan-2-yl)-6- |
| | (4-methyl-6- |
| | (trifluoromethyl)pyridin-3-yl)- |
| | 8-(pyridin-3-yl)pyrido[3,4- |
| | d]pyrimidin-4(3H)-one |
| 76 | 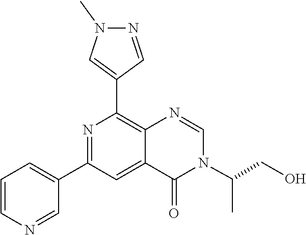 [SMILES: C[C@@H](CO)n1cnc2c(-c3cnn(C)c3)nc(-c3cccnc3)cc2c1=O] |
| | (S)-3-(1-hydroxypropan-2-yl)-8- |
| | (1-methyl-1H-pyrazol-4-yl)-6- |
| | (pyridin-3-yl)pyrido[3,4- |
| | d]pyrimidin-4(3H)-one |
| 77 | 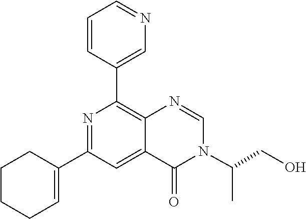 [SMILES: C[C@@H](CO)n1cnc2c(-c3cccnc3)nc(C3=CCCCC3)cc2c1=O] |
| | (S)-6-(cyclohex-1-en-1-yl)-3-(1- |
| | hydroxypropan-2-yl)-8- |
| | (pyridin-3-yl)pyrido[3,4- |
| | d]pyrimidin-4(3H)-one |
| 78 | 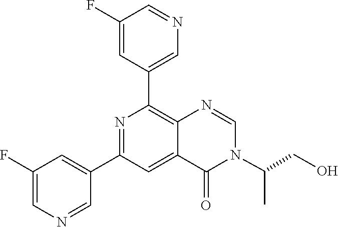 [SMILES: C[C@@H](CO)n1cnc2c(-c3cncc(F)c3)nc(-c3cncc(F)c3)cc2c1=O] |
| | (S)-6,8-bis(5-fluoropyridin-3-yl)- |
| | 3-(1-hydroxypropan-2- |
| | yl)pyrido[3,4-d]pyrimidin- |
| | 4(3H)-one |
| 79 | 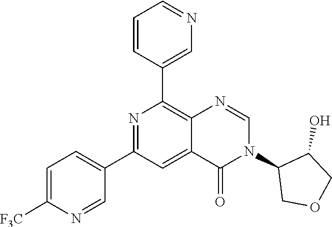 [SMILES: O=c1c2cc(-c3ccc(C(F)(F)F)nc3)nc(-c3cccnc3)c2ncn1[C@@H]1COC[C@H]1O] |
| | 3-((3R,4S)-4- |
| | hydroxytetrahydrofuran-3-yl)-8- |
| | (pyridin-3-yl)-6-(6- |
| | (trifluoromethyl)pyridin-3- |
| | yl)pyrido[3,4-d]pyrimidin- |
| | 4(3H)-one |
| 80 | 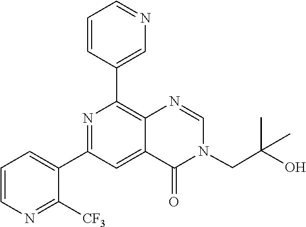 [SMILES: CC(C)(O)Cn1cnc2c(-c3cccnc3)nc(-c3cccnc3C(F)(F)F)cc2c1=O] |
| | 3-(2-hydroxy-2-methylpropyl)- |
| | 8-(pyridin-3-yl)-6-(2- |
| | (trifluoromethyl)pyridin-3- |
| | yl)pyrido[3,4-d]pyrimidin- |
| | 4(3H)-one |
| 81 | 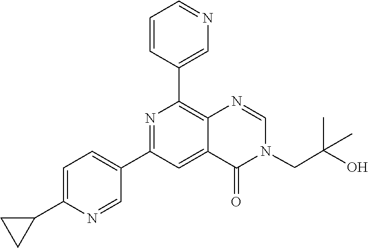 [SMILES: CC(C)(O)Cn1cnc2c(-c3cccnc3)nc(-c3ccc(C4CC4)nc3)cc2c1=O] |
| | 6-(6-cyclopropylpyridin-3-yl)- |
| | 3-(2-hydroxy-2-methylpropyl)- |
| | 8-(pyridin-3-yl)pyrido[3,4- |
| | d]pyrimidin-4(3H)-one |
| 82 | 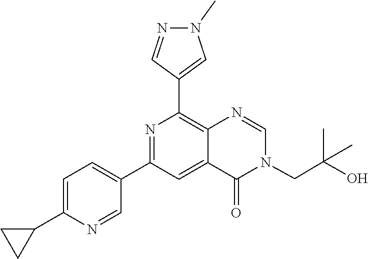 [SMILES: Cn1cc(-c2nc(-c3ccc(C4CC4)nc3)cc3c(=O)n(CC(C)(C)O)cnc23)cn1] |
| | 6-(6-cyclopropylpyridin-3-yl)-3- |
| | (2-hydroxy-2-methylpropyl)-8- |
| | (1-methyl-1H-pyrazol-4- |
| | yl)pyrido[3,4-d]pyrimidin- |
| | 4(3H)-one |
| 83 | 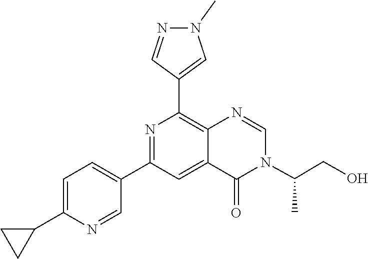 [SMILES: C[C@@H](CO)n1cnc2c(-c3cnn(C)c3)nc(-c3ccc(C4CC4)nc3)cc2c1=O] |
| | (S)-6-(6-cyclopropylpyridin-3- |
| | yl)-3-(1-hydroxypropan-2-yl)-8- |
| | (1-methyl-1H-pyrazol-4- |
| | yl)pyrido[3,4-d]pyrimidin- |
| | 4(3H)-one |
| 84 | 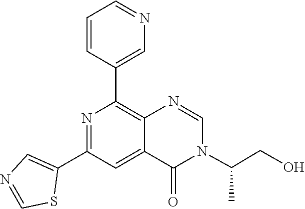 [SMILES: C[C@@H](CO)n1cnc2c(-c3cccnc3)nc(-c3cncs3)cc2c1=O] |
| | (S)-3-(1-hydroxypropan-2-yl)-8- |
| | (pyridin-3-yl)-6-(thiazol-5- |
| | yl)pyrido[3,4-d]pyrimidin- |
| | 4(3H)-one |
| 85 | 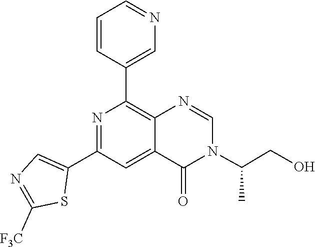 [SMILES: C[C@@H](CO)n1cnc2c(-c3cccnc3)nc(-c3cnc(C(F)(F)F)s3)cc2c1=O] |
| | (S)-3-(1-hydroxypropan-2-yl)-8- |
| | (pyridin-3-yl)-6-(2- |
| | (trifluoromethyl)thiazol-5- |
| | yl)pyrido[3,4-d]pyrimidin- |
| | 4(3H)-one |
| 86 | 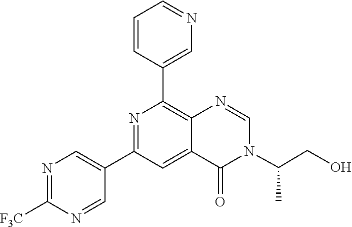 [SMILES: C[C@@H](CO)n1cnc2c(-c3cccnc3)nc(-c3cnc(C(F)(F)F)nc3)cc2c1=O] |
| | (S)-3-(1-hydroxypropan-2-yl)-8- |
| | (pyridin-3-yl)-6-(2- |
| | (trifluoromethyl)pyrimidin-5- |
| | yl)pyrido[3,4-d]pyrimidin- |
| | 4(3H)-one |
| 87 | 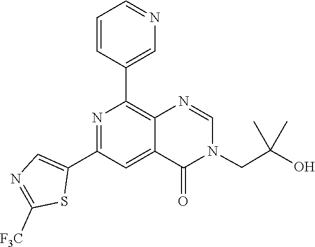 [SMILES: CC(C)(O)Cn1cnc2c(-c3cccnc3)nc(-c3cnc(C(F)(F)F)s3)cc2c1=O] |
| | 3-(2-hydroxy-2-methylpropyl)- |
| | 8-(pyridin-3-yl)-6-(2- |
| | (trifluoromethyl)thiazol-5- |
| | yl)pyrido[3,4-d]pyrimidin- |
| | 4(3H)-one |
| 88 | 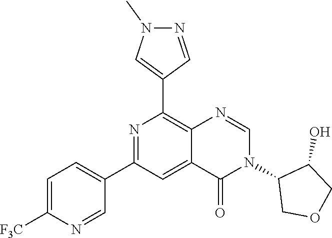 [SMILES: Cn1cc(-c2nc(-c3ccc(C(F)(F)F)nc3)cc3c(=O)n([C@H]4COC[C@H]4O)cnc23)cn1] |
| | 3-((3S,4S)-4- |
| | hydroxytetrahydrofuran-3-yl)-8- |
| | (1-methyl-1H-pyrazol-4-yl)-6- |
| | (6-(trifluoromethyl)pyridin-3- |
| | yl)pyrido[3,4-d]pyrimidin- |
| | 4(3H)-one |
| 89 | 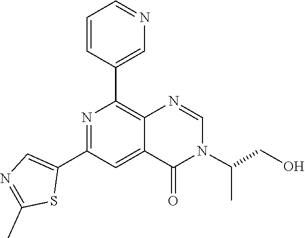 [SMILES: Cc1ncc(-c2cc3c(=O)n([C@@H](C)CO)cnc3c(-c3cccnc3)n2)s1] |
| | (S)-3-(1-hydroxypropan-2-yl)-6- |
| | (2-methylthiazol-5-yl)-8- |
| | (pyridin-3-yl)pyrido[3,4- |
| | d]pyrimidin-4(3H)-one |
| 90 | 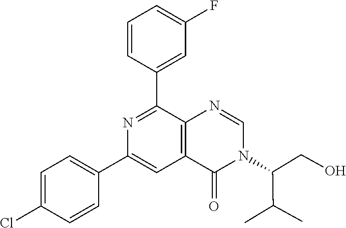 [SMILES: CC(C)[C@@H](CO)n1cnc2c(-c3cccc(F)c3)nc(-c3ccc(Cl)cc3)cc2c1=O] |
| | (S)-6-(4-chlorophenyl)-8-(3- |
| | fluorophenyl)-3-(1-hydroxy-3- |
| | methylbutan-2-yl)pyrido[3,4- |
| | d]pyrimidin-4(3H)-one |
| 91 | 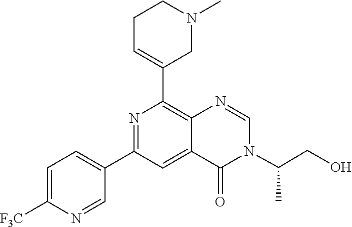 [SMILES: C[C@@H](CO)n1cnc2c(C3=CCCN(C)C3)nc(-c3ccc(C(F)(F)F)nc3)cc2c1=O] |
| | (S)-3-(1-hydroxypropan-2-yl)- |
| | 8-(1-methyl-1,2,5,6- |
| | tetrahydropyridin-3-yl)-6-(6- |
| | (trifluoromethyl)pyridin-3- |
| | yl)pyrido[3,4-d]pyrimidin- |
| | 4(3H)-one |
| 92 | 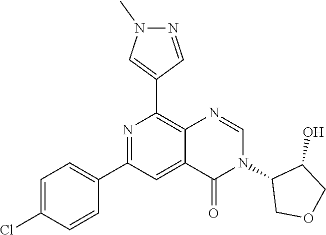 [SMILES: Cn1cc(-c2nc(-c3ccc(Cl)cc3)cc3c(=O)n([C@H]4COC[C@H]4O)cnc23)cn1] |
| | 6-(4-chlorophenyl)-3-((3S,4S)-4- |
| | hydroxytetrahydrofuran-3-yl)-8- |
| | (1-methyl-1H-pyrazol-4- |
| | yl)pyrido[3,4-d]pyrimidin- |
| | 4(3H)-one |
| 93 | 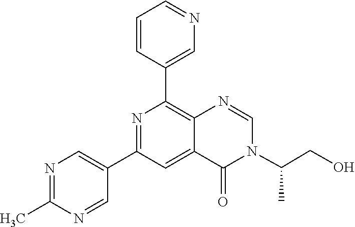 [SMILES: Cc1ncc(-c2cc3c(=O)n([C@@H](C)CO)cnc3c(-c3cccnc3)n2)cn1] |
| | (S)-3-(1-hydroxypropan-2-yl)- |
| | 6-(2-methylpyrimidin-5-yl)-8- |
| | (pyridin-3-yl)pyrido[3,4- |
| | d]pyrimidin-4(3H)-one |
| 94 | 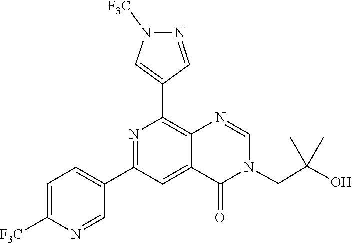 [SMILES: CC(C)(O)Cn1cnc2c(-c3cnn(C(F)(F)F)c3)nc(-c3ccc(C(F)(F)F)nc3)cc2c1=O] |
| | 3-(2-hydroxy-2-methylpropyl)- |
| | 8-(1-(trifluoromethyl)-1H- |
| | pyrazol-4-yl)-6-(6- |
| | (trifluoromethyl)pyridin-3- |
| | yl)pyrido[3,4-d]pyrimidin- |
| | 4(3H)-one |
| 95 | 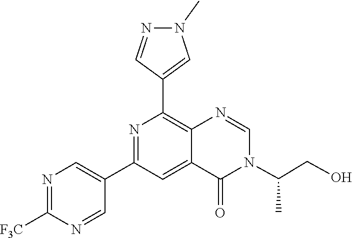 [SMILES: C[C@@H](CO)n1cnc2c(-c3cnn(C)c3)nc(-c3cnc(C(F)(F)F)nc3)cc2c1=O] |
| | (S)-3-(1-hydroxypropan-2-yl)-8- |
| | (1-methyl-1H-pyrazol-4-yl)-6- |
| | (2-(trifluoromethyl)pyrimidin-5- |
| | yl)pyrido[3,4-d]pyrimidin- |
| | 4(3H)-one |
| 96 | 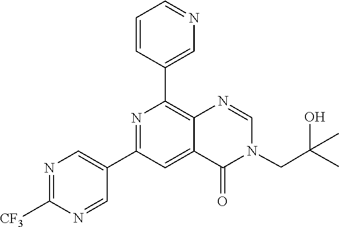 [SMILES: CC(C)(O)Cn1cnc2c(-c3cccnc3)nc(-c3cnc(C(F)(F)F)nc3)cc2c1=O] |
| | 3-(2-hydroxy-2-methylpropyl)- |
| | 8-(pyridin-3-yl)-6-(2- |
| | (trifluoromethyl)pyrimidin-5- |
| | yl)pyrido[3,4-d]pyrimidin- |
| | 4(3H)-one |
| 97 | 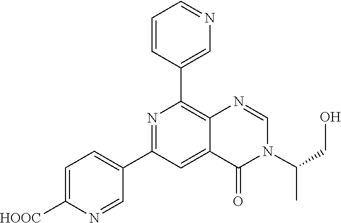 [SMILES: C[C@@H](CO)n1cnc2c(-c3cccnc3)nc(-c3ccc(C(=O)O)nc3)cc2c1=O] |
| | (S)-5-(3-(1-hydroxypropan-2- |
| | yl)-4-oxo-8-(pyridin-3-yl)-3,4- |
| | dihydropyrido[3,4-d]pyrimidin- |
| | 6-yl)picolinic acid |
| 98 | 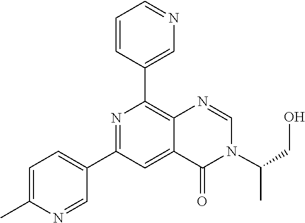 [SMILES: Cc1ccc(-c2cc3c(=O)n([C@@H](C)CO)cnc3c(-c3cccnc3)n2)cn1] |
| | (S)-3-(1-hydroxypropan-2-yl)-6- |
| | (6-methylpyridin-3-yl)-8- |
| | (pyridin-3-yl)pyrido[3,4- |
| | d]pyrimidin-4(3H)-one |
| 99 | 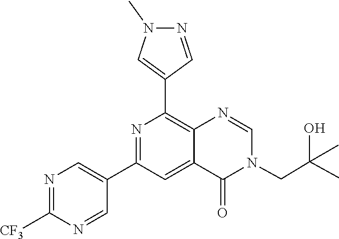 [SMILES: Cn1cc(-c2nc(-c3cnc(C(F)(F)F)nc3)cc3c(=O)n(CC(C)(C)O)cnc23)cn1] |
| | 3-(2-hydroxy-2-methylpropyl)-8- |
| | (1-methyl-1H-pyrazol-4-yl)-6-(2- |
| | (trifluoromethyl)pyrimidin-5-yl) |
| | pyrido[3,4-d]pyrimidin-4(3H)-one |
| 100 | 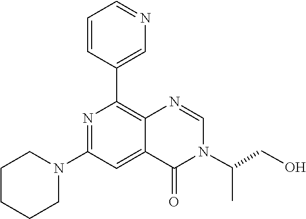 [SMILES: C[C@@H](CO)n1cnc2c(-c3cccnc3)nc(N3CCCCC3)cc2c1=O] |
| | (S)-3-(1-hydroxypropan-2-yl)-6- |
| | (piperidin-1-yl)-8-(pyridin-3- |
| | yl)pyrido[3,4-d]pyrimidin- |
| | 4(3H)-one |
| 101 | 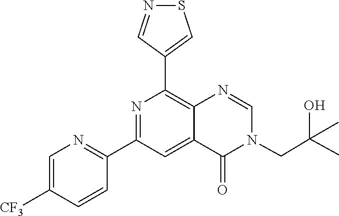 [SMILES: CC(C)(O)Cn1cnc2c(-c3cnsc3)nc(-c3ccc(C(F)(F)F)cn3)cc2c1=O] |
| | 3-(2-hydroxy-2-methylpropyl)- |
| | 8-( isothiazol-4-yl)-6-(5- |
| | (trifluoromethyl)pyridin-2- |
| | yl)pyrido[3,4-d]pyrimidin- |
| | 4(3H)-one |
| 102 | 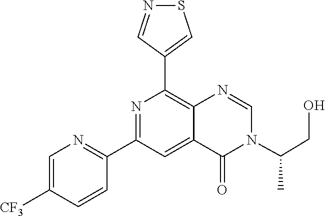 [SMILES: C[C@@H](CO)n1cnc2c(-c3cnsc3)nc(-c3ccc(C(F)(F)F)cn3)cc2c1=O] |
| | (S)-3-(1-hydroxypropan-2-yl)- |
| | 8-( isothiazol-4-yl)-6-(5- |
| | (trifluoromethyl)pyridin-2- |
| | yl)pyrido[3,4-d]pyrimidin- |
| | 4(3H)-one |
| 103 | 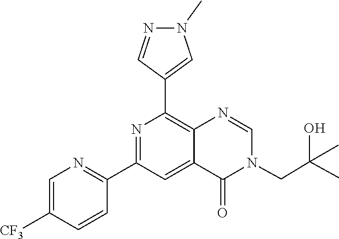 [SMILES: Cn1cc(-c2nc(-c3ccc(C(F)(F)F)cn3)cc3c(=O)n(CC(C)(C)O)cnc23)cn1] |
| | 3-(2-hydroxy-2-methylpropyl)- |
| | 8-(1-methyl-1H-pyrazol-4-yl)-6- |
| | (5-(trifluoromethyl)pyridin-2- |
| | yl)pyrido[3,4-d]pyrimidin- |
| | 4(3H)-one |
| 104 | 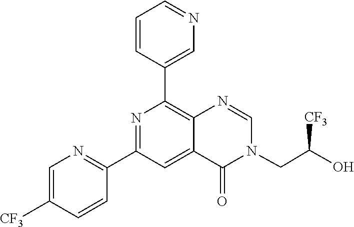 [SMILES: O=c1c2cc(-c3ccc(C(F)(F)F)cn3)nc(-c3cccnc3)c2ncn1C[C@H](O)C(F)(F)F] |
| | (S)-8-(pyridin-3-yl)-3-(3,3,3- |
| | trifluoro-2-hydroxypropyl)-6- |
| | (5-(trifluoromethyl)pyridin-2- |
| | yl)pyrido[3,4-d]pyrimidin- |
| | 4(3H)-one |
| 105 | 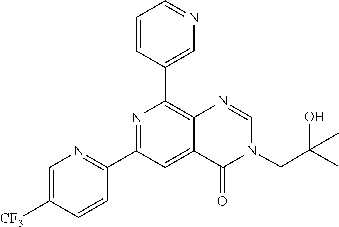 [SMILES: CC(C)(O)Cn1cnc2c(-c3cccnc3)nc(-c3ccc(C(F)(F)F)cn3)cc2c1=O] |
| | (3-(2-hydroxy-2-methylpropyl)- |
| | 8-(pyridin-3-yl)-6-(5- |
| | (trifluoromethyl)pyridin-2- |
| | yl)pyrido[3,4-d]pyrimidin- |
| | 4(3H)-one |
| 106 | 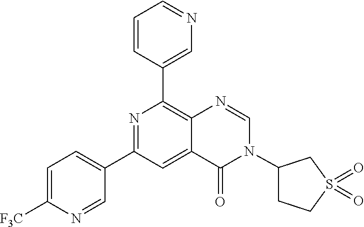 [SMILES: O=c1c2cc(-c3ccc(C(F)(F)F)nc3)nc(-c3cccnc3)c2ncn1C1CCS(=O)(=O)C1] |
| | 3-(1,1- |
| | dioxidotetrahydrothiophen-3- |
| | yl)-8-(pyridin-3-yl)-6-(6- |
| | (trifluoromethyl)pyridin-3- |
| | yl)pyrido[3,4-d]pyrimidin- |
| | 4(3H)-one |
| 107 | 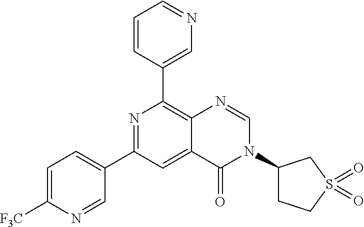 [SMILES: O=c1c2cc(-c3ccc(C(F)(F)F)nc3)nc(-c3cccnc3)c2ncn1[C@@H]1CCS(=O)(=O)C1] |
| | (R)-3-(1,1- |
| | dioxidotetrahydrothiophen-3-yl)- |
| | 8-(pyridin-3-yl)-6-(6- |
| | (trifluoromethyl)pyridin-3- |
| | yl)pyrido[3,4-d]pyrimidin- |
| | 4(3H)-one |
| 108 | 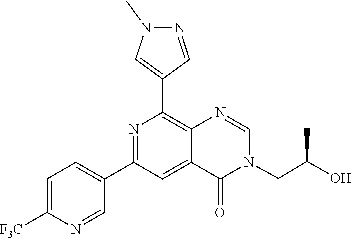 [SMILES: C[C@@H](O)Cn1cnc2c(-c3cnn(C)c3)nc(-c3ccc(C(F)(F)F)nc3)cc2c1=O] |
| | (R)-3-(2-hydroxypropyl)-8-(1- |
| | methyl-1H-pyrazol-4-yl)-6-(6- |
| | (trifluoromethyl)pyridin-3- |
| | yl)pyrido[3,4-d]pyrimidin- |
| | 4(3H)-one |
| 1090 | 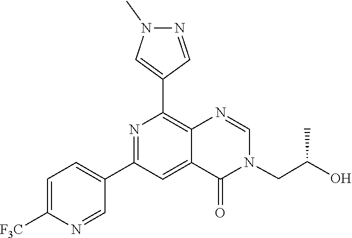 [SMILES: C[C@H](O)Cn1cnc2c(-c3cnn(C)c3)nc(-c3ccc(C(F)(F)F)nc3)cc2c1=O] |
| | (S)-3-(2-hydroxypropyl)-8-(1- |
| | methyl-1H-pyrazol-4-yl)-6-(6- |
| | (trifluoromethyl)pyridin-3- |
| | yl)pyrido[3,4-d]pyrimidin- |
| | 4(3H)-one |
| 110 | 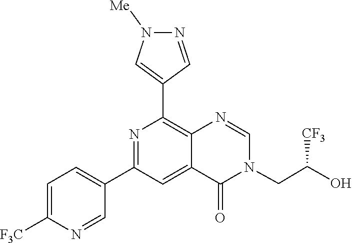 [SMILES: Cn1cc(-c2nc(-c3ccc(C(F)(F)F)nc3)cc3c(=O)n(C[C@@H](O)C(F)(F)F)cnc23)cn1] |
| | (R)-8-(1-methyl-1H-pyrazol-4- |
| | yl)-3-(3,3,3-trifluoro-2- |
| | hydroxypropyl)-6-(6- |
| | (trifluoromethyl)pyridin-3- |
| | yl)pyrido[3,4-d]pyrimidin- |
| | 4(3H)-one |
| 111 | 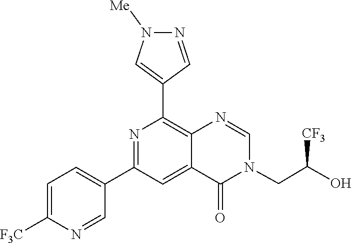 [SMILES: Cn1cc(-c2nc(-c3ccc(C(F)(F)F)nc3)cc3c(=O)n(C[C@H](O)C(F)(F)F)cnc23)cn1] |
| | (S)-8-(1-methyl-1H-pyrazol-4- |
| | yl)-3-(3,3,3-trifluoro-2- |
| | hydroxypropyl)-6-(6- |
| | (trifluoromethyl)pyridin-3- |
| | yl)pyrido[3,4-d]pyrimidin- |
| | 4(3H)-one |
| 112 | 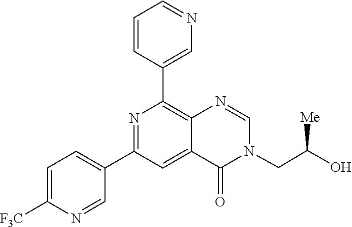 [SMILES: C[C@@H](O)Cn1cnc2c(-c3cccnc3)nc(-c3ccc(C(F)(F)F)nc3)cc2c1=O] |
| | (R)-3-(2-hydroxypropyl)-8- |
| | (pyridin-3-yl)-6-(6- |
| | (trifluoromethyl)pyridin-3- |
| | yl)pyrido[3,4-d]pyrimidin- |
| | 4(3H)-one |
| 113 | 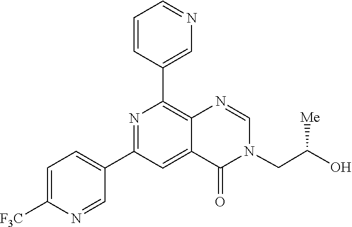 [SMILES: C[C@H](O)Cn1cnc2c(-c3cccnc3)nc(-c3ccc(C(F)(F)F)nc3)cc2c1=O] |
| | (R)-8-(1-methyl-1H-pyrazol-4- |
| | yl)-3-(3,3,3-trifluoro-2- |
| | hydroxypropyl)-6-(5- |
| | (trifluoromethyl)pyridin-2- |
| | yl)pyrido[3,4-d]pyrimidin- |
| | 4(3H)-one |
| 114 | 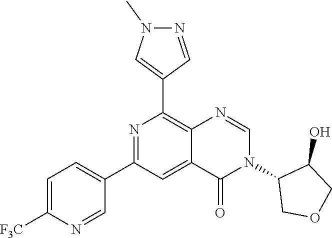 [SMILES: Cn1cc(-c2nc(-c3ccc(C(F)(F)F)nc3)cc3c(=O)n([C@H]4COC[C@@H]4O)cnc23)cn1] |
| | 3-((3S,4R)-4- |
| | hydroxytetrahydrofuran-3-yl)-8- |
| | (1-methyl-1H-pyrazol-4-yl)-6- |
| | (6-(trifluoromethyl)pyridin-3- |
| | yl)pyrido[3,4-d]pyrimidin- |
| | 4(3H)-one |
| 115 | 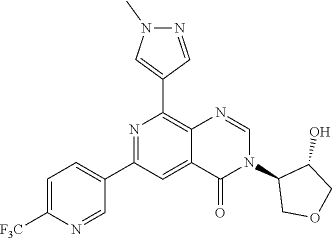 [SMILES: Cn1cc(-c2nc(-c3ccc(C(F)(F)F)nc3)cc3c(=O)n([C@@H]4COC[C@H]4O)cnc23)cn1] |
| | 3-((3R,4S)-4- |
| | hydroxytetrahydrofuran-3-yl)-8- |
| | (1-methyl-1H-pyrazol-4-yl)-6- |
| | (6-(trifluoromethyl)pyridin-3- |
| | yl)pyrido[3,4-d]pyrimidin- |
| | 4(3H)-one |
| 116 | 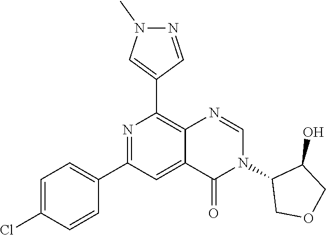 [SMILES: Cn1cc(-c2nc(-c3ccc(Cl)cc3)cc3c(=O)n([C@H]4COC[C@@H]4O)cnc23)cn1] |
| | 6-(4-chlorophenyl)-3-((3S,4R)- |
| | 4-hydroxytetrahydrofuran-3- |
| | yl)-8-(1-methyl-1H-pyrazol-4- |
| | yl)pyrido[3,4-d]pyrimidin- |
| | 4(3H)-one |
| 117 | 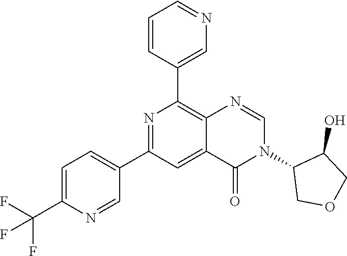 [SMILES: O=c1c2cc(-c3ccc(C(F)(F)F)nc3)nc(-c3cccnc3)c2ncn1[C@H]1COC[C@@H]1O] |
| | 3-((3S,4R)-4- |
| | hydroxytetrahydrofuran-3-yl)-8- |
| | (pyridin-3-yl)-6-(6- |
| | (trifluoromethyl)pyridin-3- |
| | yl)pyrido[3,4-d]pyrimidin- |
| | 4(3H)-one |
| 118 | 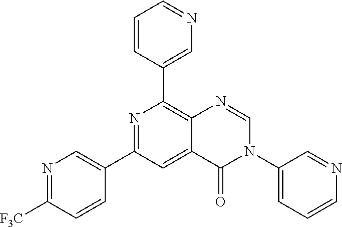 [SMILES: O=c1c2cc(-c3ccc(C(F)(F)F)nc3)nc(-c3cccnc3)c2ncn1-c1cccnc1] |
| | 3,8-di(pyridin-3-yl)-6-(6- |
| | (trifluoromethyl)pyridin-3- |
| | yl)pyrido[3,4-d]pyrimidin- |
| | 4(3H)-one |
| 119 | 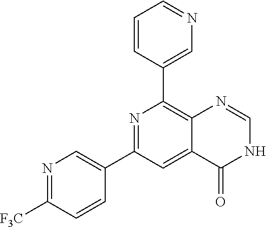 [SMILES: O=c1[nH]cnc2c(-c3cccnc3)nc(-c3ccc(C(F)(F)F)nc3)cc12] |
| | 8-(pyridin-3-yl)-6-(6- |
| | (trifluoromethyl)pyridin-3- |
| | yl)pyrido[3,4-d]pyrimidin- |
| | 4(3H)-one |
| 120 | 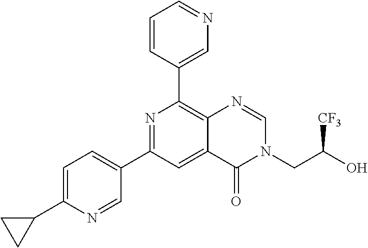 [SMILES: O=c1c2cc(-c3ccc(C4CC4)nc3)nc(-c3cccnc3)c2ncn1C[C@H](O)C(F)(F)F] |
| | (S)-6-(6-cyclopropylpyridin-3- |
| | yl)-8-(pyridin-3-yl)-3-(3,3,3- |
| | trifluoro-2- |
| | hydroxypropyl)pyrido[3,4- |
| | d]pyrimidin-4(3H)-one |
| 121 | 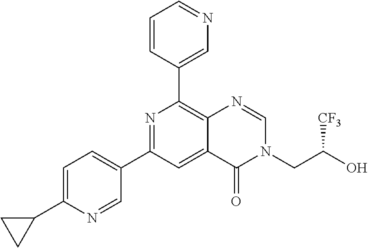 [SMILES: O=c1c2cc(-c3ccc(C4CC4)nc3)nc(-c3cccnc3)c2ncn1C[C@@H](O)C(F)(F)F] |
| | (R)-6-(6-cyclopropylpyridin-3- |
| | yl)-8-(pyridin-3-yl)-3-(3,3,3- |
| | trifluoro-2- |
| | hydroxypropyl)pyrido[3,4- |
| | d]pyrimidin-4(3H)-one |
| 122 | 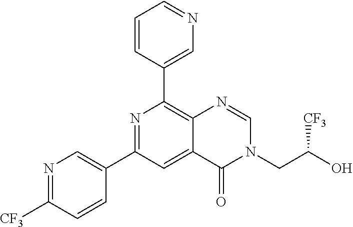 [SMILES: O=c1c2cc(-c3ccc(C(F)(F)F)nc3)nc(-c3cccnc3)c2ncn1C[C@@H](O)C(F)(F)F] |
| | trifluoro-2-hydroxypropyl)-6-(6- |
| | (trifluoromethyl)pyridin-3- |
| | yl)pyrido[3,4-d]pyrimidin- |
| | 4(3H)-one |
| 123 | 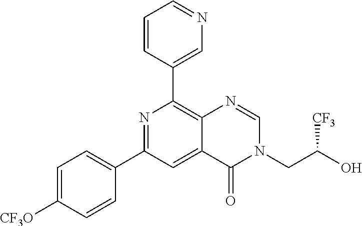 |
| | (R)-8-(pyridin-3-yl)-3-(3,3,3- |
| | trifluoro-2-hydroxypropyl)-6-(4- |
| | (trifluoromethoxy)phenyl)pyrido |
| | [3,4-d]pyrimidin-4(3H)-one |
| 124 | 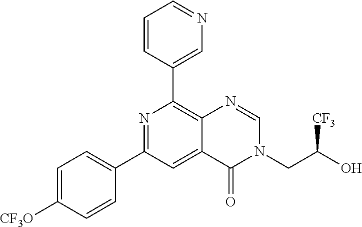 |
| | (S)-8-(pyridin-3-yl)-3-(3,3,3- |
| | trifluoro-2-hydroxypropyl)-6-(4- |
| | (trifluoromethoxy)phenyl)pyrido |
| | [3,4-d]pyrimidin-4(3H)-one |
| 125 | 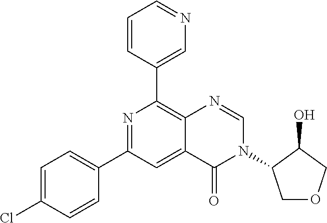 [SMILES: O=c1c2cc(-c3ccc(Cl)cc3)nc(-c3cccnc3)c2ncn1[C@H]1COC[C@@H]1O] |
| | 6-(4-chlorophenyl)-3-((3S,4R)-4- |
| | hydroxytetrahydrofuran-3-yl)-8- |
| | (pyridin-3-yl)pyrido[3,4- |
| | d]pyrimidin-4(3H)-one |
| 126 | 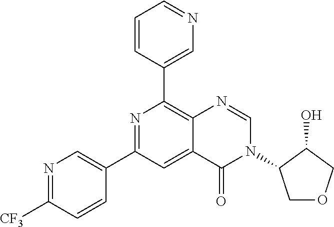 [SMILES: O=c1c2cc(-c3ccc(C(F)(F)F)nc3)nc(-c3cccnc3)c2ncn1[C@H]1COC[C@H]1O] |
| | 3-((3S,4S)-4- |
| | hydroxytetrahydrofuran-3-yl)-8- |
| | (pyridin-3-yl)-6-(6- |
| | (trifluoromethyl)pyridin-3- |
| | yl)pyrido[3,4-d]pyrimidin- |
| | 4(3H)-one |
| 127 | 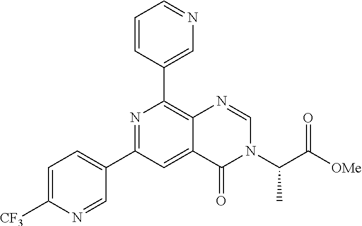 [SMILES: COC(=O)[C@H](C)n1cnc2c(-c3cccnc3)nc(-c3ccc(C(F)(F)F)nc3)cc2c1=O] |
| | methyl (S)-2-(4-oxo-8-(pyridin- |
| | 3-yl)-6-(6- |
| | (trifluoromethyl)pyridin-3- |
| | yl)pyrido[3,4-d]pyrimidin- |
| | 3(4H)-yl)propanoate |
| 128 | 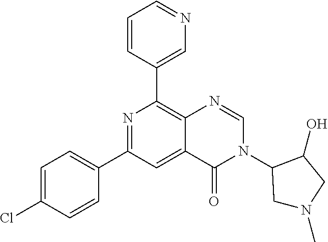 [SMILES: CN1CC(O)C(n2cnc3c(-c4cccnc4)nc(-c4ccc(Cl)cc4)cc3c2=O)C1] |
| | 6-(4-chlorophenyl)-3-(4- |
| | hydroxy-1-methylpyrrolidin-3- |
| | yl)-8-(pyridin-3-yl)pyrido[3,4- |
| | d]pyrimidin-4(3H)-one |
| 129 | 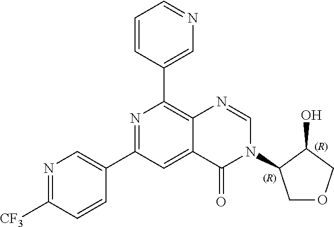 |
| | 3-((3R,4R)-4- |
| | hydroxytetrahydrofuran-3-yl)-8- |
| | (pyridin-3-yl)-6-(6- |
| | (trifluoromethyl)pyridin-3- |
| | yl)pyrido[3,4-d]pyrimidin- |
| | 4(3H)-one |
| 130 | 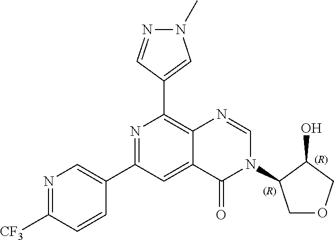 |
| | 3-((3R,4R)-4- |
| | hydroxytetrahydrofuran-3-yl)-8- |
| | (1-methyl-1H-pyrazol-4-yl)-6- |
| | (6-(trifluoromethyl)pyridin-3- |
| | yl)pyrido[3,4-d]pyrimidin- |
| | 4(3H)-one |
| 131 | 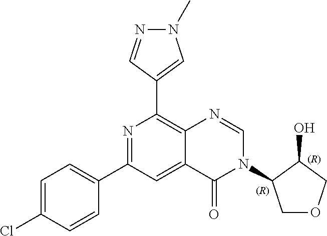 |
| | 6-(4-chlorophenyl)-3-((3R,4R)- |
| | 4-hydroxytetrahydrofuran-3-yl)- |
| | 8-(1-methyl-1H-pyrazol-4- |
| | yl)pyrido[3,4-d]pyrimidin- |
| | 4(3H)-one |
| 132 | 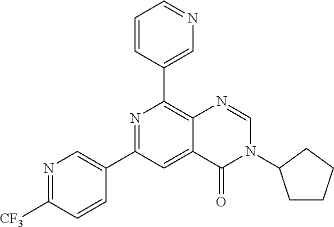 [SMILES: O=c1c2cc(-c3ccc(C(F)(F)F)nc3)nc(-c3cccnc3)c2ncn1C1CCCC1] |
| | 3-cyclopentyl-8-(pyridin-3-yl)- |
| | 6-(6-(trifluoromethyl)pyridin-3- |
| | yl)pyrido[3,4-d]pyrimidin- |
| | 4(3H)-one |
| 133 | 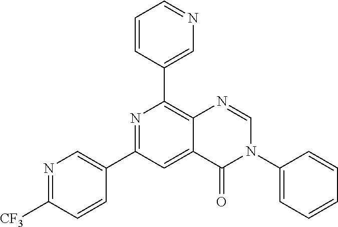 [SMILES: O=c1c2cc(-c3ccc(C(F)(F)F)nc3)nc(-c3cccnc3)c2ncn1-c1ccccc1] |
| | 3-phenyl-8-(pyridin-3-yl)-6-(6- |
| | (trifluoromethyl)pyridin-3- |
| | yl)pyrido[3,4-d]pyrimidin- |
| | 4(3H)-one |
| 134 | 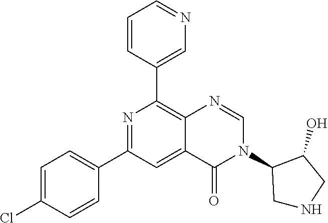 [SMILES: O=c1c2cc(-c3ccc(Cl)cc3)nc(-c3cccnc3)c2ncn1[C@@H]1CNC[C@H]1O] |
| | 6-(4-chlorophenyl)-3-((3R,4R)- |
| | 4-hydroxypyrrolidin-3-yl)-8- |
| | (pyridin-3-yl)pyrido[3,4-d] |
| | pyrimidin-4(3H)-one |
| 135 | 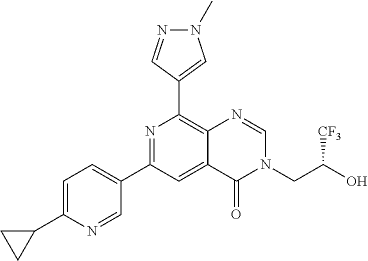 [SMILES: Cn1cc(-c2nc(-c3ccc(C4CC4)nc3)cc3c(=O)n(C[C@@H](O)C(F)(F)F)cnc23)cn1] |
| | (R)-6-(6-cyclopropylpyridin-3- |
| | yl)-8-(1-methyl-1H-pyrazol-4- |
| | yl)-3-(3,3,3-trifluoro-2- |
| | hydroxypropyl)pyrido[3,4- |
| | d]pyrimidin-4(3H)-one |
| 136 | 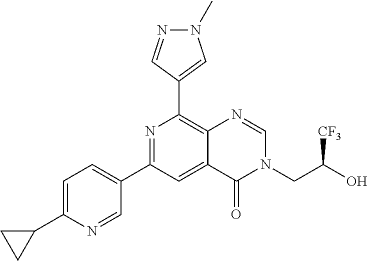 [SMILES: Cn1cc(-c2nc(-c3ccc(C4CC4)nc3)cc3c(=O)n(C[C@H](O)C(F)(F)F)cnc23)cn1] |
| | (S)-6-(6-cyclopropylpyridin-3- |
| | yl)-8-(1-methyl-1/-pyrazol-4- |
| | yl)-3-(3,3,3-trifluoro-2- |
| | hydroxypropyl)pyrido[3,4- |
| | d]pyrimidin-4(3H)-one |
| 137 | 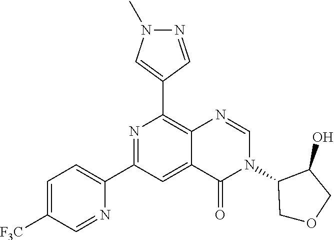 [SMILES: Cn1cc(-c2nc(-c3ccc(C(F)(F)F)cn3)cc3c(=O)n([C@H]4COC[C@@H]4O)cnc23)cn1] |
| | 3-((3S,4R)-4- |
| | hydroxytetrahydrofuran-3-yl)- |
| | 8-(1-methyl-1H-pyrazol-4-yl)- |
| | 6-(5-(trifluoromethyl)pyridin- |
| | 2-yl)pyrido[3,4-d]pyrimidin- |
| | 4(3H)-one |
| 138 | 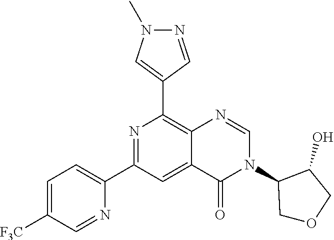 [SMILES: Cn1cc(-c2nc(-c3ccc(C(F)(F)F)cn3)cc3c(=O)n([C@@H]4COC[C@H]4O)cnc23)cn1] |
| | 3-((3R,4S)-4- |
| | hydroxytetrahydrofuran-3-yl)-8- |
| | (1-methyl-1H-pyrazol-4-yl)-6- |
| | (5-(trifluoromethyl)pyridin-2- |
| | yl)pyrido[3,4-d]pyrimidin- |
| | 4(3H)-one |
| 139 | 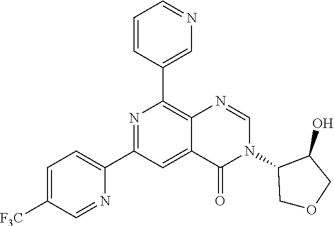 [SMILES: O=c1c2cc(-c3ccc(C(F)(F)F)cn3)nc(-c3cccnc3)c2ncn1[C@H]1COC[C@@H]1O] |
| | 3-((3S,4R)-4- |
| | hydroxytetrahydrofuran-3-yl)-8- |
| | (pyridin-3-yl)-6-(5- |
| | (trifluoromethyl)pyridin-2- |
| | yl)pyrido[3,4-d]pyrimidin-4(3H)- |
| | one |
| 140 | 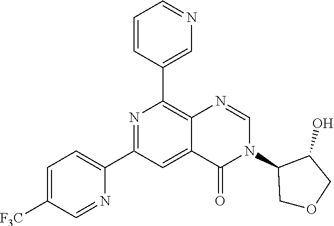 [SMILES: O=c1c2cc(-c3ccc(C(F)(F)F)cn3)nc(-c3cccnc3)c2ncn1[C@@H]1COC[C@H]1O] |
| | 3-((3R,4S)-4- |
| | hydroxytetrahydrofuran-3-yl)-8- |
| | (pyridin-3-yl)-6-(5- |
| | (trifluoromethyl)pyridin-2- |
| | yl)pyrido[3,4-d]pyrimidin- |
| | 4(3H)-one |
| 141 | 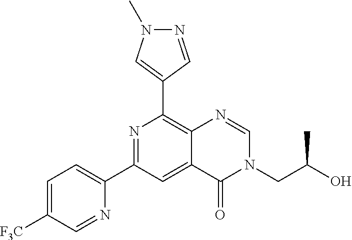 [SMILES: C[C@@H](O)Cn1cnc2c(-c3cnn(C)c3)nc(-c3ccc(C(F)(F)F)cn3)cc2c1=O] |
| | (R)-3-(2-hydroxypropyl)-8-(1- |
| | methyl-1H-pyrazol-4-yl)-6-(5- |
| | (trifluoromethyl)pyridin-2- |
| | yl)pyrido[3,4-d]pyrimidin-4(3H)- |
| | one |
| 142 | 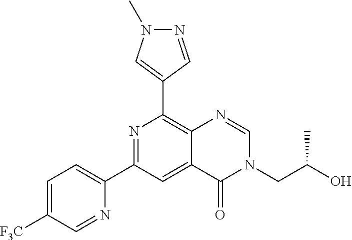 [SMILES: C[C@H](O)Cn1cnc2c(-c3cnn(C)c3)nc(-c3ccc(C(F)(F)F)cn3)cc2c1=O] |
| | (S)-3-(2-hydroxypropyl)-8-(1- |
| | methyl-1H-pyrazol-4-yl)-6-(5- |
| | (trifluoromethyl)pyridin-2- |
| | yl)pyrido[3,4-d]pyrimidin- |
| | 4(3H)-one |
| 143 | 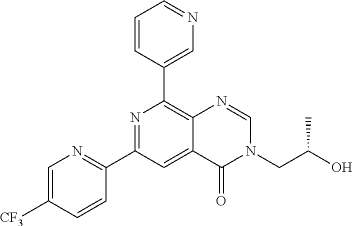 [SMILES: C[C@H](O)Cn1cnc2c(-c3cccnc3)nc(-c3ccc(C(F)(F)F)cn3)cc2c1=O] |
| | (S)-3-(2-hydroxypropyl)-8- |
| | (pyridin-3-yl)-6-(5- |
| | (trifluoromethyl)pyridin-2- |
| | yl)pyrido[3,4-d]pyrimidin-4(3H)- |
| | one |
| 144 | 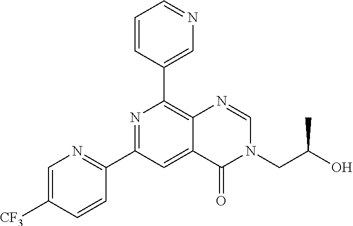 [SMILES: C[C@@H](O)Cn1cnc2c(-c3cccnc3)nc(-c3ccc(C(F)(F)F)cn3)cc2c1=O] |
| | (R)-3-(2-hydroxypropyl)-8- |
| | (pyridin-3-yl)-6-(5- |
| | (trifluoromethyl)pyridin-2- |
| | yl)pyrido[3,4-d]pyrimidin- |
| | 4(3H)-one |
| 145 | 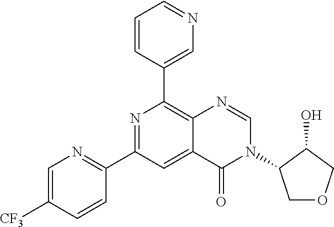 [SMILES: O=c1c2cc(-c3ccc(C(F)(F)F)cn3)nc(-c3cccnc3)c2ncn1[C@H]1COC[C@H]1O] |
| | 3-((3S,4S)-4- |
| | hydroxytetrahydrofuran-3-yl)-8- |
| | (pyridin-3-yl)-6-(5- |
| | (trifluoromethyl)pyridin-2- |
| | yl)pyrido[3,4-d]pyrimidin-4(3H)- |
| | one |
| 146 | 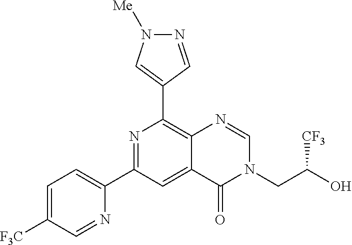 [SMILES: Cn1cc(-c2nc(-c3ccc(C(F)(F)F)cn3)cc3c(=O)n(C[C@@H](O)C(F)(F)F)cnc23)cn1] |
| | (S)-3-(2-hydroxypropyl)-8- |
| | (pyridin-3-yl)-6-(6- |
| | (trifluoromethyl)pyridin-3- |
| | yl)pyrido[3,4-d]pyrimidin- |
| | 4(3H)-one |
| 147 | 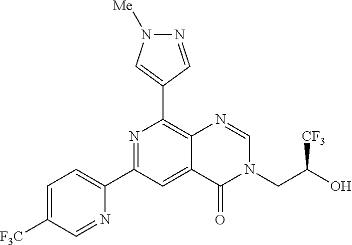 [SMILES: Cn1cc(-c2nc(-c3ccc(C(F)(F)F)cn3)cc3c(=O)n(C[C@H](O)C(F)(F)F)cnc23)cn1] |
| | (S)-8-(1-methyl-1H-pyrazol-4- |
| | yl)-3-(3,3,3-trifluoro-2- |
| | hydroxypropyl)-6-(5- |
| | (trifluoromethyl)pyridin-2- |
| | yl)pyrido[3,4-d]pyrimidin-4(3H)- |
| | one |
| 148 | 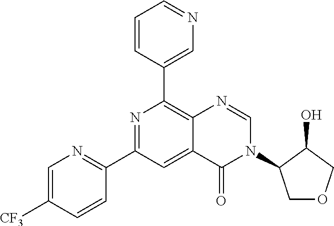 [SMILES: O=c1c2cc(-c3ccc(C(F)(F)F)cn3)nc(-c3cccnc3)c2ncn1[C@@H]1COC[C@@H]1O] |
| | 3-((3R,4R)-4- |
| | hydroxytetrahydrofuran-3-yl)-8- |
| | (pyridin-3-yl)-6-(5- |
| | (trifluoromethyl)pyridin-2- |
| | yl)pyrido[3,4-d]pyrimidin- |
| | 4(3H)-one |
| 149 | 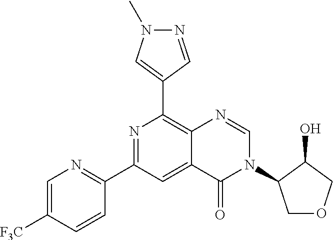 [SMILES: Cn1cc(-c2nc(-c3ccc(C(F)(F)F)cn3)cc3c(=O)n([C@@H]4COC[C@@H]4O)cnc23)cn1] |
| | 3-((3R,4R)-4- |
| | hydroxytetrahydrofuran-3-yl)-8- |
| | (1-methyl-1H-pyrazol-4-yl)-6-(5- |
| | (trifluoromethyl)pyridin-2- |
| | yl)pyrido[3,4-d]pyrimidin-4(3H)- |
| | one |
| 150 | 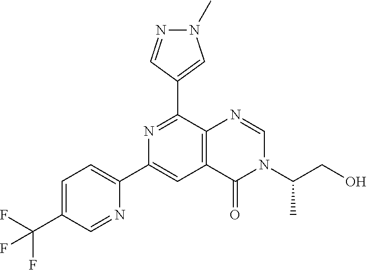 [SMILES: C[C@@H](CO)n1cnc2c(-c3cnn(C)c3)nc(-c3ccc(C(F)(F)F)cn3)cc2c1=O] |
| | (S)-3-(1-hydroxypropan-2-yl)-8- |
| | (1-methyl-1H-pyrazol-4-yl)-6- |
| | (5-(trifluoromethyl) |
| | pyridin-2-yl)pyrido[3,4- |
| | d]pyrimidin-4(3H)-one |
| 151 | 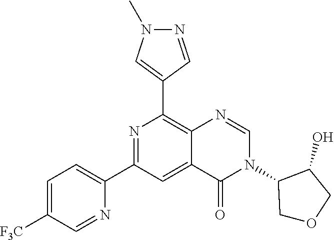 [SMILES: Cn1cc(-c2nc(-c3ccc(C(F)(F)F)cn3)cc3c(=O)n([C@H]4COC[C@H]4O)cnc23)cn1] |
| | 3-((3S,4S)-4- |
| | hydroxytetrahydrofuran-3-yl)-8- |
| | (1-methyl-1H-pyrazol-4-yl)-6- |
| | (5-(trifluoromethyl)pyridin-2- |
| | yl)pyrido[3,4-d]pyrimidin- |
| | 4(3H)-one |
| 152 | 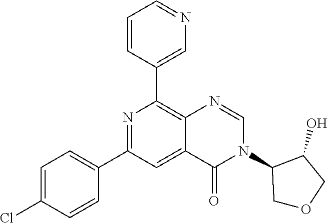 [SMILES: O=c1c2cc(-c3ccc(Cl)cc3)nc(-c3cccnc3)c2ncn1[C@@H]1COC[C@H]1O] |
| | 6-(4-chlorophenyl)-3-((3R,4S)-4- |
| | hydroxytetrahydrofuran-3-yl)-8- |
| | (pyridin-3-yl)pyrido[3,4- |
| | d]pyrimidin-4(3H)-one |
| 153 | 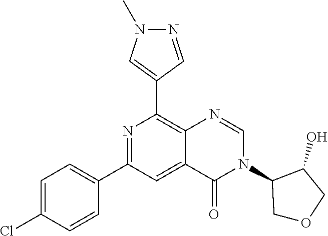 [SMILES: Cn1cc(-c2nc(-c3ccc(Cl)cc3)cc3c(=O)n([C@@H]4COC[C@H]4O)cnc23)cn1] |
| | 6-(4-Chlorophenyl)-3-((3R,4S)- |
| | 4-hydroxytetrahydrofuran-3-yl)- |
| | 8-(1-methyl-1H-pyrazol-4- |
| | yl)pyrido[3,4-d]pyrimidin- |
| | 4(3H)-one |
| 154 | 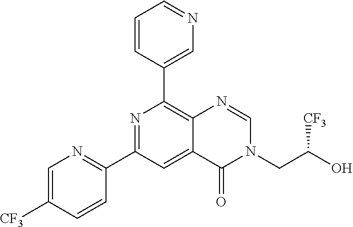 [SMILES: O=c1c2cc(-c3ccc(C(F)(F)F)cn3)nc(-c3cccnc3)c2ncn1C[C@@H](O)C(F)(F)F] |
| | (R)-8-(pyridin-3-yl)-3-(3,3,3- |
| | trifluoro-2-hydroxypropyl)-6-(5- |
| | (trifluoromethyl)pyridin-2- |
| | yl)pyrido[3,4-d]pyrimidin- |
| | 4(3H)-one |
| 155 | 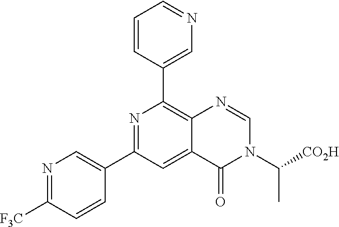 [SMILES: C[C@@H](C(=O)O)n1cnc2c(-c3cccnc3)nc(-c3ccc(C(F)(F)F)nc3)cc2c1=O] |
| | (S)-2-(4-oxo-8-(pyridin-3-yl)- |
| | 6-(6-(trifluoromethyl)pyridin- |
| | 3-yl)pyrido[3,4-d]pyrimidin- |
| | 3(4H)-yl)propanoic acid |
| 156 | 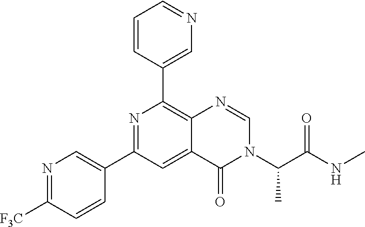 [SMILES: CNC(=O)[C@H](C)n1cnc2c(-c3cccnc3)nc(-c3ccc(C(F)(F)F)nc3)cc2c1=O] |
| | (S)-N-methyl-2-(4-oxo-8- |
| | (pyridin-3-yl)-6-(6- |
| | (trifluoromethyl)pyridin-3- |
| | yl)pyrido[3,4-d]pyrimidin- |
| | 3(4H)-yl)propanamide |
| 157 | 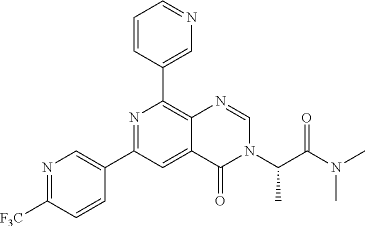 [SMILES: C[C@@H](C(=O)N(C)C)n1cnc2c(-c3cccnc3)nc(-c3ccc(C(F)(F)F)nc3)cc2c1=O] |
| | (S)-N,N-dimethyl-2-(4-oxo-8- |
| | (pyridin-3-yl)-6-(6- |
| | (trifluoromethyl)pyridin-3- |
| | yl)pyrido[3,4-d]pyrimidin- |
| | 3(4H)-yl)propenamide |
| 158 | 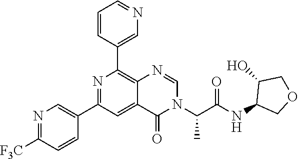 [SMILES: C[C@@H](C(=O)N[C@@H]1COC[C@H]1O)n1cnc2c(-c3cccnc3)nc(-c3ccc(C(F)(F)F)nc3)cc2c1=O] |
| | (S)-N-((3R,4S)-4- |
| | hydroxytetrahydrofuran-3-yl)-2- |
| | (4-oxo-8-(pyridin-3-yl)-6-(6- |
| | (trifluoromethyl)pyridin-3- |
| | yl)pyrido[3,4-d]pyrimidin- |
| | 3(4H)-yl)propanamide |
| 159 | 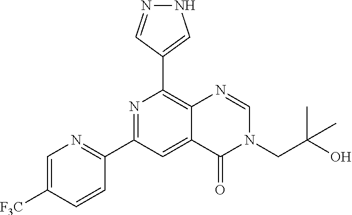 [SMILES: CC(C)(O)Cn1cnc2c(-c3cn[nH]c3)nc(-c3ccc(C(F)(F)F)cn3)cc2c1=O] |
| | 3-(2-hydroxy-2-methylpropyl)-8- |
| | (1H-pyrazol-4-yl)-6-(5- |
| | (trifluoromethyl)pyridin-2-yl)pyrido |
| | [3,4-d]pyrimidin-4(3H)-one |
| 160 | 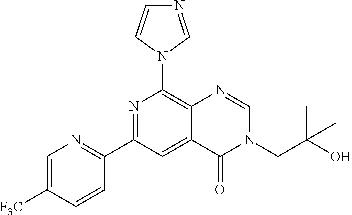 [SMILES: CC(C)(O)Cn1cnc2c(-n3ccnc3)nc(-c3ccc(C(F)(F)F)cn3)cc2c1=O] |
| | 3-(2-hydroxy-2-methylpropyl)- |
| | 8-(1H-imidazol-1-yl)-6-(5- |
| | (trifluoromethyl)pyridin-2- |
| | yl)pyrido[3,4-d]pyrimidin- |
| | 4(3H)-one |
| 161 | 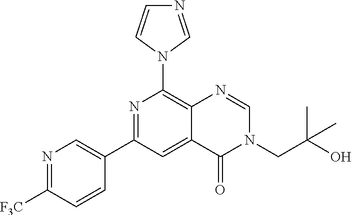 [SMILES: CC(C)(O)Cn1cnc2c(-n3ccnc3)nc(-c3ccc(C(F)(F)F)nc3)cc2c1=O] |
| | 3-(2-hydroxy-2-methylpropyl)- |
| | 8-(1H-imidazol-1-yl)-6-(6- |
| | (trifluoromethyl)pyridin-2- |
| | yl)pyrido[3,4-d]pyrimidin- |
| | 4(3H)-one |
| 162 | 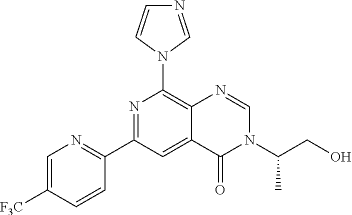 [SMILES: C[C@@H](CO)n1cnc2c(-n3ccnc3)nc(-c3ccc(C(F)(F)F)cn3)cc2c1=O] |
| | (S)-3-(1-hydroxypropan-2-yl)- |
| | 8-(1H-imidazol-1-yl)-6-(5- |
| | (trifluoromethyl)pyridin-2- |
| | yl)pyrido[3,4-d]pyrimidin- |
| | 4(3H)-one |
| 163 | 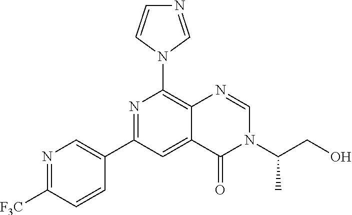 [SMILES: C[C@@H](CO)n1cnc2c(-n3ccnc3)nc(-c3ccc(C(F)(F)F)nc3)cc2c1=O] |
| | (S)-3-(1-hydroxypropan-2-yl)-8- |
| | (1H-imidazol-1-yl)-6-(6- |
| | (trifluoromethyl)pyridin-3- |
| | yl)pyrido[3,4-d]pyrimidin- |
| | 4(3H)-one |
| 164 | 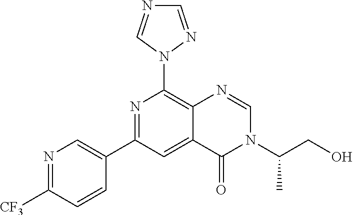 [SMILES: C[C@@H](CO)n1cnc2c(-n3cncn3)nc(-c3ccc(C(F)(F)F)nc3)cc2c1=O] |
| | (S)-3-(1-hydroxypropan-2-yl)-8- |
| | (1H-1,2,4-triazol-1-yl)-6-(6- |
| | (trifluoromethyl)pyridin-3- |
| | yl)pyrido[3,4-d]pyrimidin- |
| | 4(3H)-one |
| 165 | 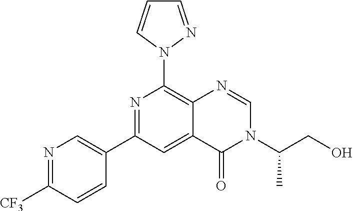 [SMILES: C[C@@H](CO)n1cnc2c(-n3cccn3)nc(-c3ccc(C(F)(F)F)nc3)cc2c1=O] |
| | (S)-3-(1-hydroxypropan-2-yl)-8- |
| | (1H-pyrazol-1-yl)-6-(6- |
| | (trifluoromethyl)pyridin-3- |
| | yl)pyrido[3,4-d]pyrimidin- |
| | 4(3H)-one |
| 166 | 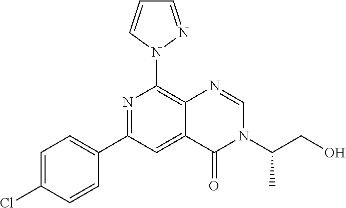 [SMILES: C[C@@H](CO)n1cnc2c(-n3cccn3)nc(-c3ccc(Cl)cc3)cc2c1=O] |
| | (S)-6-(4-chlorophenyl)-3-(1- |
| | hydroxypropan-2-yl)-8-(1H- |
| | pyrazol-1-yl)pyrido[3,4- |
| | d]pyrimidin-4(3H)-one |
| 167 | 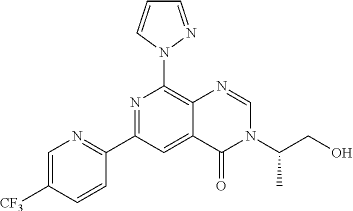 [SMILES: C[C@@H](CO)n1cnc2c(-n3cccn3)nc(-c3ccc(C(F)(F)F)cn3)cc2c1=O] |
| | (S)-3-(1-hydroxypropan-2-yl)-8- |
| | (1H-pyrazol-1-yl)-6-(5- |
| | (trifluoromethyl)pyridin-2- |
| | yl)pyrido[3,4-d]pyrimidin- |
| | 4(3H)-one |
| 168 | 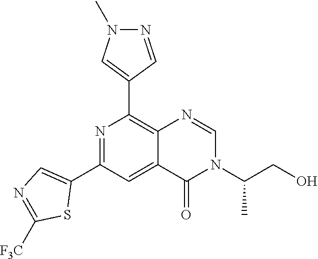 [SMILES: C[C@@H](CO)n1cnc2c(-c3cnn(C)c3)nc(-c3cnc(C(F)(F)F)s3)cc2c1=O] |
| | S)-3-(1-hydroxypropan-2-yl)- |
| | 8-(1-methyl-1H-pyrazol-4-yl)- |
| | 6-(2-(trifluoromethyl)thiazol-5- |
| | yl)pyrido[3,4-d]pyrimidin- |
| | 4(3H)-one |
| 169 | 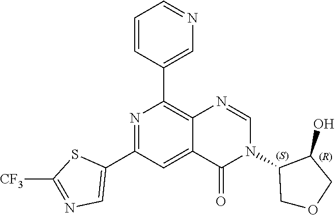 |
| | 3-((3S,4R)-4- |
| | hydroxytetrahydrofuran-3-yl)-8- |
| | (pyridin-3-yl)-6-(2- |
| | (trifluoromethyl)thiazol-5- |
| | yl)pyrido[3,4-d]pyrimidin- |
| | 4(3H)-one |
| 170 | 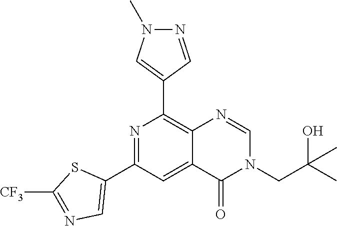 [SMILES: Cn1cc(-c2nc(-c3cnc(C(F)(F)F)s3)cc3c(=O)n(CC(C)(C)O)cnc23)cn1] |
| | 3-(2-hydroxy-2- |
| | methylpropyl)-8-(1-methyl- |
| | 1H-pyrazol-4-yl)-6-(2- |
| | (trifluoromethyl)thiazol-5- |
| | yl)pyrido[3,4-d]pyrimidin- |
| | 4(3H)-one |
| 171 | 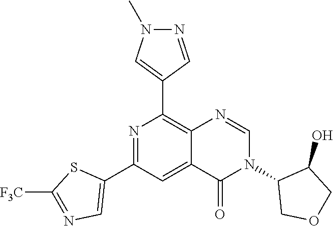 [SMILES: Cn1cc(-c2nc(-c3cnc(C(F)(F)F)s3)cc3c(=O)n([C@H]4COC[C@@H]4O)cnc23)cn1] |
| | 3-((3S,4R)-4- |
| | hydroxytetrahydrofuran-3-yl)-8- |
| | (1-methyl-1H-pyrazol-4-yl)-6- |
| | (2-(trifluoromethyl)thiazol-5- |
| | yl)pyrido[3,4-d]pyrimidin- |
| | 4(3H)-one |
| 172 | 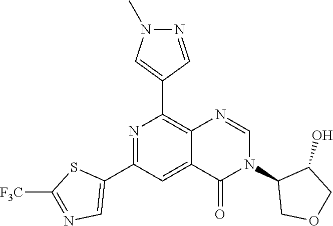 [SMILES: Cn1cc(-c2nc(-c3cnc(C(F)(F)F)s3)cc3c(=O)n([C@@H]4COC[C@H]4O)cnc23)cn1] |
| | 3-((3R,4S)-4- |
| | hydroxytetrahydrofuran-3-yl)- |
| | 8-(1-methyl-1H-pyrazol-4-yl)- |
| | 6-(2-(trifluoromethyl)thiazol-5- |
| | yl)pyrido[3,4-d]pyrimidin- |
| | 4(3H)-one |
| 173 | 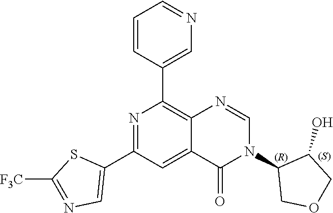 |
| | 3-((3R,4S)-4- |
| | hydroxytetrahydrofuran-3-yl)-8- |
| | (pyridin-3-yl)-6-(2- |
| | (trifluoromethyl)thiazol-5- |
| | yl)pyrido[3,4-d]pyrimidin- |
| | 4(3H)-one |
| 174 | 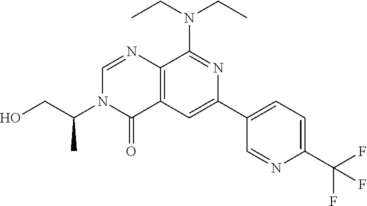 [SMILES: CCN(CC)c1nc(-c2ccc(C(F)(F)F)nc2)cc2c(=O)n([C@@H](C)CO)cnc12] |
| | (S)-8-(diethylamino)-3-(1- |
| | hydroxypropan-2-yl)-6-(6- |
| | (trifluoromethyl)pyridin- |
| | 3-yl)pyrido[3,4-d]pyrimidin- |
| | 4(3H)-one |
| 175 | 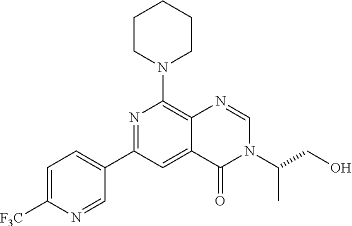 [SMILES: C[C@@H](CO)n1cnc2c(N3CCCCC3)nc(-c3ccc(C(F)(F)F)nc3)cc2c1=O] |
| | (S)-3-(1-hydroxypropan-2-yl)-8- |
| | (piperidin-1-yl)-6-(6- |
| | (trifluoromethyl)pyridin-3- |
| | yl)pyrido[3,4-d]pyrimidin- |
| | 4(3H)-one |
| 176 | 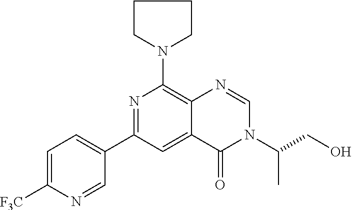 [SMILES: C[C@@H](CO)n1cnc2c(N3CCCC3)nc(-c3ccc(C(F)(F)F)nc3)cc2c1=O] |
| | (S)-3-(1-hydroxypropan-2-yl)-8- |
| | (pyrrolidin-1-yl)-6-(6- |
| | (trifluoromethyl)pyridin-3- |
| | yl)pyrido[3,4-d]pyrimidin- |
| | 4(3H)-one |
| 177 | 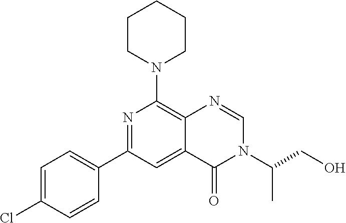 [SMILES: C[C@@H](CO)n1cnc2c(N3CCCCC3)nc(-c3ccc(Cl)cc3)cc2c1=O] |
| | (S)-6-(4-chlorophenyl)-3-(1- |
| | hydroxypropan-2-yl)-8- |
| | (piperidin-1-yl)pyrido[3,4- |
| | d]pyrimidin-4(3H)-one |
| 178 | 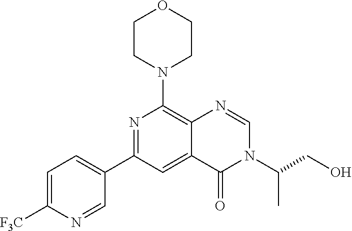 [SMILES: C[C@@H](CO)n1cnc2c(N3CCOCC3)nc(-c3ccc(C(F)(F)F)nc3)cc2c1=O] |
| | (S)-3-(1-hydroxypropan-2-yl)-8- |
| | morpholino-6-(6- |
| | (trifluoromethyl)pyridin-3- |
| | yl)pyrido[3,4-d]pyrimidin- |
| | 4(3H)-one |
| 179 | 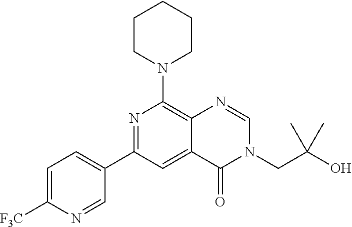 [SMILES: CC(C)(O)Cn1cnc2c(N3CCCCC3)nc(-c3ccc(C(F)(F)F)nc3)cc2c1=O] |
| | 3-(2-hydroxy-2-methylpropyl)- |
| | 8-(piperidin-1-yl)-6-(6- |
| | (trifluoromethyl)pyridin-3- |
| | yl)pyrido[3,4-d]pyrimidin- |
| | 4(3H)-one |
| 180 | 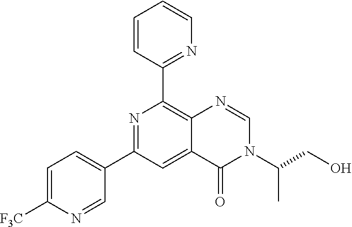 [SMILES: C[C@@H](CO)n1cnc2c(-c3ccccn3)nc(-c3ccc(C(F)(F)F)nc3)cc2c1=O] |
| | (S)-3-(1-hydroxypropan-2-yl)-8- |
| | (pyridin-2-yl)-6-(6- |
| | (trifluoromethyl)pyridin-3- |
| | yl)pyrido[3,4-d]pyrimidin- |
| | 4(3H)-one |
| 181 | 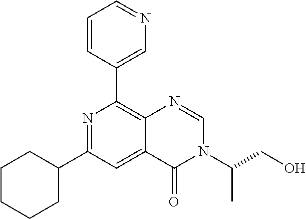 [SMILES: C[C@@H](CO)n1cnc2c(-c3cccnc3)nc(C3CCCCC3)cc2c1=O] |
| | (S)-6-cyclohexyl-3-(1- |
| | hydroxypropan-2-yl)-8- |
| | (pyridin-3-yl)pyrido[3,4- |
| | d]pyrimidin-4(3H)-one |
| 182 | 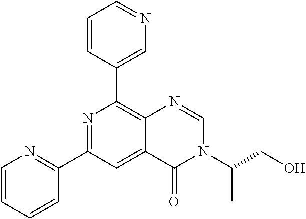 [SMILES: C[C@@H](CO)n1cnc2c(-c3cccnc3)nc(-c3ccccn3)cc2c1=O] |
| | (S)-3-(1-hydroxypropan-2-yl)-6- |
| | (pyridin-2-yl)-8-(pyridin-3- |
| | yl)pyrido[3,4-d]pyrimidin- |
| | 4(3H)-one |
| 183 | 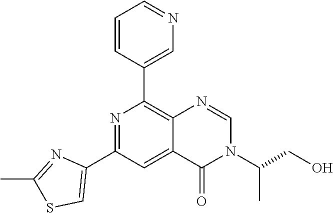 [SMILES: Cc1nc(-c2cc3c(=O)n([C@@H](C)CO)cnc3c(-c3cccnc3)n2)cs1] |
| | (S)-3-(1-hydroxypropan-2-yl)-6- |
| | (2-methylthiazol-4-yl)-8- |
| | (pyridin-3-yl)pyrido[3,4- |
| | d]pyrimidin-4(3H)-one |
| 184 | 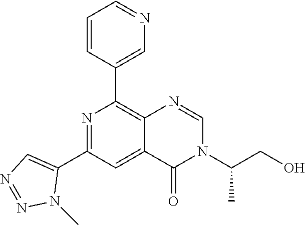 [SMILES: C[C@@H](CO)n1cnc2c(-c3cccnc3)nc(-c3cnnn3C)cc2c1=O] |
| | (S)-3-(1-hydroxypropan-2-yl)-6- |
| | (1-methyl-1H-1,2,3-triazol-5-yl)- |
| | 8-(pyridin-3-yl)pyrido[3,4- |
| | d]pyrimidin-4(3H)-one |
| 185 enan- tiomer 1 and 185 enan- tiomer 2 | 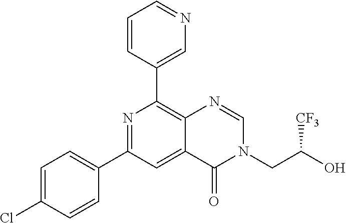 [SMILES: O=c1c2cc(-c3ccc(Cl)cc3)nc(-c3cccnc3)c2ncn1C[C@@H](O)C(F)(F)F] |
| | 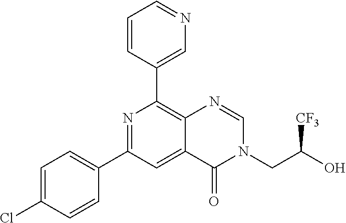 [SMILES: O=c1c2cc(-c3ccc(Cl)cc3)nc(-c3cccnc3)c2ncn1C[C@H](O)C(F)(F)F] |
| | (R)-6-(4-chlorophenyl)-8-(pyridin-3- |
| | yl)-3-(3,3,3-trifluoro-2-hydroxypropyl) |
| | pyrido[3,4-d]pyrimidin-4(3H)-one |
| | and |
| | (S)-6-(4-chlorophenyl)-8-(pyridin-3- |
| | yl)-3-(3,3,3-trifluoro-2-hydroxypropyl) |
| | pyrido[3,4-d]pyrimidin-4(3H)-one |
| 186 | 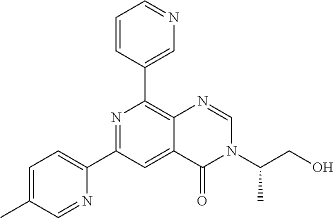 [SMILES: Cc1ccc(-c2cc3c(=O)n([C@@H](C)CO)cnc3c(-c3cccnc3)n2)nc1] |
| | (S)-3-(1-hydroxypropan-2-yl)- |
| | 6-(5-methylpyridin-2-yl)-8- |
| | (pyridin-3-yl)pyrido[3,4- |
| | d]pyrimidin-4(3H)-one |
| 187 | 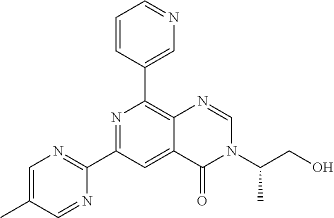 [SMILES: Cc1cnc(-c2cc3c(=O)n([C@@H](C)CO)cnc3c(-c3cccnc3)n2)nc1] |
| | (S)-3-(1-hydroxypropan-2-yl)-6- |
| | (5-methylpyrimidin-2-yl)-8- |
| | (pyridin-3-yl)pyrido[3,4- |
| | d]pyrimidin-4(3H)-one |
| 188 | 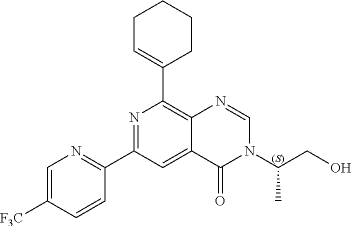 |
| | (S)-8-(cyclohex-1-en-1-yl)-3- |
| | (1-hydroxypropan-2-yl)-6-(5- |
| | (trifluoromethyl)pyridin-2- |
| | yl)pyrido[3,4-d]pyrimidin- |
| | 4(3H)-one |
| 189 | 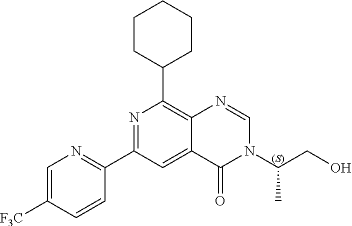 |
| | (S)-8-cyclohexyl-3-(1- |
| | hydroxypropan-2-yl)-6-(5- |
| | (trifluoromethyl)pyridin-2- |
| | yl)pyrido[3,4-d]pyrimidin- |
| | 4(3H)-one |
| 190 | 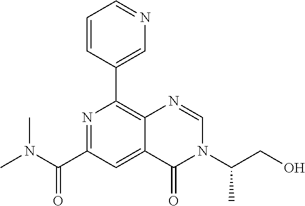 [SMILES: C[C@@H](CO)n1cnc2c(-c3cccnc3)nc(C(=O)N(C)C)cc2c1=O] |
| | (S)-3-(1-hydroxypropan-2-yl)- |
| | N,N-dimethyl-4-oxo-8- |
| | (pyridin-3-yl)-3,4- |
| | dihydropyrido[3,4- |
| | d]pyrimidine-6-carboxamide |
| 191 | 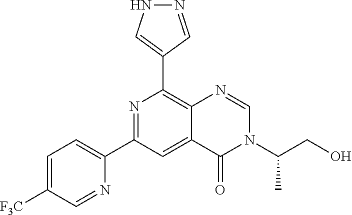 [SMILES: C[C@@H](CO)n1cnc2c(-c3cn[nH]c3)nc(-c3ccc(C(F)(F)F)cn3)cc2c1=O] |
| | (S)-3-(1-hydroxypropan-2-yl)-8- |
| | (1H-pyrazol-4-yl)-6-(5- |
| | (trifluoromethyl)pyridin-2- |
| | yl)pyrido[3,4-d]pyrimidin- |
| | 4(3H)-one |
| 192 | 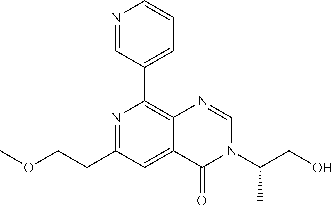 [SMILES: COCCc1cc2c(=O)n([C@@H](C)CO)cnc2c(-c2cccnc2)n1] |
| | (S)-3-(1-hydroxypropan-2-yl)- |
| | 6-(2-methoxyethyl)-8-(pyridin- |
| | 3-yl)pyrido[3,4-d]pyrimidin- |
| | 4(3H)-one |
| 193 | 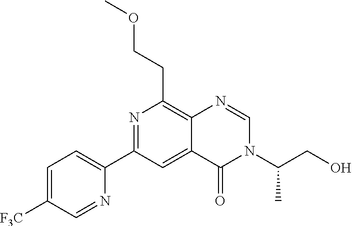 [SMILES: COCCc1nc(-c2ccc(C(F)(F)F)cn2)cc2c(=O)n([C@@H](C)CO)cnc12] |
| | (S)-3-(1-hydroxypropan-2-yl)-8- |
| | (2-methoxyethyl)-6-(5- |
| | (trifluoromethyl)pyridin-2- |
| | yl)pyrido[3,4-d]pyrimidin- |
| | 4(3H)-one |

In some embodiments, an AhR antagonist is chosen from the compounds below and pharmaceutically acceptable salts thereof:
(i) (S)-8-(5-fluoropyridin-3-yl)-3-(1-hydroxypropan-2-yl)-6-(4-(trifluoromethoxy)phenyl)pyrido[3,4-d]pyrimidin-4(3H)-one;
(ii) (S)-6-(4-chlorophenyl)-3-(1-hydroxypropan-2-yl)-8-(6-oxo-1,6-dihydropyridin-3-yl)pyrido[3,4-d]pyrimidin-4(3H)-one;
(iii)(S)-8-(benzo[d][1,3]dioxol-4-yl)-6-(4-chlorophenyl)-3-(1-hydroxypropan-2-yl)pyrido[3,4-d]pyrimidin-4(3H)-one;
(iv)(S)-3-(1-hydroxypropan-2-yl)-8-(pyridin-4-yl)-6-(6-(trifluoromethyl)pyridin-3-yl)pyrido[3,4-d]pyrimidin-4(3H)-one;
(v) (S)-8-(5-fluoropyridin-3-yl)-3-(1-hydroxypropan-2-yl)-6-(4-(trifluoromethyl)phenyl)pyrido[3,4-d]pyrimidin-4(3H)-one;
(vi)(S)-3-(1-hydroxypropan-2-yl)-6,8-di(pyridin-4-yl)pyrido[3,4-d]pyrimidin-4(3H)-one;
(vii) (S)-6-(4-chlorophenyl)-8-(3-fluorophenyl)-3-(1-hydroxypropan-2-yl)pyrido[3,4-d]pyrimidin-4(3H)-one;
(viii) (S)-8-(3-fluorophenyl)-3-(1-hydroxypropan-2-yl)-6-(6-(trifluoromethyl)pyridin-3-yl)pyrido[3,4-d]pyrimidin-4(3H)-one;
(ix) 3-(2-hydroxy-2-methylpropyl)-8-(pyridin-3-yl)-6-(6-(trifluoromethyl)pyridin-3-yl)pyrido[3,4-d]pyrimidin-4(3H)-one;
(x) 6,8-di(pyridin-3-yl)-3-(3,3,3-trifluoro-2-hydroxypropyl)pyrido[3,4-d]pyrimidin-4(3H)-one;
(xi)(S)-6-chloro-3-(1-hydroxypropan-2-yl)-8-(pyridin-3-yl)pyrido[3,4-d]pyrimidin-4(3H)-one;
(xii) (S)-3-(1-hydroxypropan-2-yl)-8-(pyridin-3-yl)-6-(6-(trifluoromethyl)pyridin-3-yl)pyrido[3,4-d]pyrimidin-4(3H)-one;
(xiii) 6-(4-chlorophenyl)-8-(pyridin-3-yl)-3-(3,3,3-trifluoro-2-hydroxypropyl)pyrido[3,4-d]pyrimidin-4(3H)-one;
(xiv) 8-(pyridin-3-yl)-3-(3,3,3-trifluoro-2-hydroxypropyl)-6-(4-(trifluoromethoxy)phenyl)pyrido[3,4-d]pyrimidin-4(3H)-one;
(xv) 6-(4-chlorophenyl)-8-(1-methyl-1H-pyrazol-4-yl)-3-(3,3,3-trifluoro-2-hydroxypropyl)pyrido[3,4-d]pyrimidin-4(3H)-one;
(xvi) 3-(2-hydroxy-2-methylpropyl)-8-(pyridin-3-yl)-6-(4-(trifluoromethyl)phenyl)pyrido[3,4-d]pyrimidin-4(3H)-one;
(xvii) (S)-3-(1-hydroxypropan-2-yl)-8-(pyridin-3-yl)-6-(p-tolyl)pyrido[3,4-d]pyrimidin-4(3H)-one;
(xviii) 6-(4-chlorophenyl)-3-(2-hydroxy-2-methylpropyl)-8-(pyridin-3-yl)pyrido[3,4-d]pyrimidin-4(3H)-one;
(xix) 3-(2-hydroxy-2-methylpropyl)-6,8-bis(1-methyl-1H-pyrazol-4-yl)pyrido[3,4-d]pyrimidin-4(3H)-one;
(xx) (S)-3-(1-hydroxypropan-2-yl)-6,8-di(pyridin-3-yl)pyrido[3,4-d]pyrimidin-4(3H)-one;
(xxi) 6-(4-chlorophenyl)-3-(2-hydroxy-2-methylpropyl)-8-(1-methyl-1H-pyrazol-4-yl)pyrido[3,4-d]pyrimidin-4(3H)-one;
(xxii) (S)-3-(1-hydroxypropan-2-yl)-8-(pyridin-3-yl)-6-(4-(trifluoromethoxy)phenyl)pyrido[3,4-d]pyrimidin-4(3H)-one;
(xxiii) (S)-3-(1-hydroxypropan-2-yl)-8-(pyridin-3-yl)-6-(4-(trifluoromethyl)phenyl)pyrido[3,4-d]pyrimidin-4(3H)-one;
(xxiv) (S)-6-(4-chlorophenyl)-3-(1-hydroxypropan-2-yl)-8-(pyridin-3-yl)pyrido[3,4-d]pyrimidin-4(3H)-one;
(xxv) (S)-3-(1-hydroxypropan-2-yl)-8-(1-methyl-1H-pyrazol-4-yl)-6-(4-(trifluoromethoxy)phenyl)pyrido[3,4-d]pyrimidin-4(3H)-one;
(xxvi) (S)-3-(1-hydroxypropan-2-yl)-8-(1-methyl-1H-pyrazol-4-yl)-6-phenylpyrido[3,4-d]pyrimidin-4(3H)-one;
(xxvii) (S)-6-(4-chlorophenyl)-3-(1-hydroxypropan-2-yl)-8-(1-methyl-1H-pyrazol-4-yl)pyrido[3,4-d]pyrimidin-4(3H)-one;
(xxviii) 3-methyl-8-(pyridin-3-yl)-6-(4-(trifluoromethoxy)phenyl)pyrido[3,4-d]pyrimidin-4(3H)-one;
(xxix) Rac-6-(4-chlorophenyl)-3-((trans)-4-hydroxytetrahydrofuran-3-yl)-8-(pyridin-3-yl)pyrido[3,4-d]pyrimidin-4(3H)-one;
(xxx) (S)-6-(4-chlorophenyl)-3-(3-hydroxy-3-methylbutan-2-yl)-8-(1-methyl-1H-pyrazol-4-yl)pyrido[3,4-d]pyrimidin-4(3H)-one;
(xxxi) (R)-6-(4-chlorophenyl)-3-(3-hydroxy-3-methylbutan-2-yl)-8-(1-methyl-1H-pyrazol-4-yl)pyrido[3,4-d]pyrimidin-4(3H)-one;
(xxxii) rac-6-(4-chlorophenyl)-3-((cis)-4-hydroxytetrahydrofuran-3-yl)-8-(pyridin-3-yl)pyrido[3,4-d]pyrimidin-4(3H)-one;
(xxxiii) (R)-6-(4-chlorophenyl)-3-(3-hydroxy-3-methylbutan-2-yl)-8-(pyridin-3-yl)pyrido[3,4-d]pyrimidin-4(3H)-one;
(xxxiv) (S)-3-(3-hydroxy-3-methylbutan-2-yl)-6,8-di(pyridin-3-yl)pyrido[3,4-d]pyrimidin-4(3H)-one;
(xxxv) (S)-6,8-bis(3,5-difluorophenyl)-3-(1-hydroxy-3-methylbutan-2-yl)pyrido[3,4-d]pyrimidin-4(3H)-one;
(xxxvi) (S)-6-(4-chlorophenyl)-3-(3-hydroxy-3-methylbutan-2-yl)-8-(pyridin-3-yl)pyrido[3,4-d]pyrimidin-4(3H)-one;
(xxxvii) (S)-8-(3,5-difluorophenyl)-3-(1-hydroxy-3-methylbutan-2-yl)-6-(p-tolyl)pyrido[3,4-d]pyrimidin-4(3H)-one;
(xxxviii) 6-(4-chlorophenyl)-8-(3-fluorophenyl)-3-(2-hydroxy-2-methylpropyl)pyrido[3,4-d]pyrimidin-4(3H)-one;
(xxxix) (R)-6-(4-chlorophenyl)-8-(3-fluorophenyl)-3-(3-hydroxy-3-methylbutan-2-yl)pyrido[3,4-d]pyrimidin-4(3H)-one;
(xl)(S)-3-(1-(benzyloxy)propan-2-yl)-8-(3-fluorophenyl)-6-(p-tolyl)pyrido[3,4-d]pyrimidin-4(3H)-one;
(xli) (R)-6-(4-chlorophenyl)-8-(3-fluorophenyl)-3-(3,3,3-trifluoro-2-hydroxypropyl)pyrido[3,4-d]pyrimidin-4(3H)-one;
(xlii) (S)-6-(4-chlorophenyl)-8-(3-fluorophenyl)-3-(3,3,3-trifluoro-2-hydroxypropyl)pyrido[3,4-d]pyrimidin-4(3H)-one;
(xliii) (S)-3-(1-hydroxypropan-2-yl)-6-morpholino-8-(pyridin-3-yl)pyrido[3,4-d]pyrimidin-4(3H)-one;
(xliv) (S)-3-(1-hydroxypropan-2-yl)-8-(1H-imidazol-1-yl)-6-(4-(trifluoromethoxy)phenyl)pyrido[3,4-d]pyrimidin-4(3H)-one;
(xlv) (S)-3-(1-methoxypropan-2-yl)-8-(pyridin-3-yl)-6-(p-tolyl)pyrido[3,4-d]pyrimidin-4(3H)-one;
(xlvi) (S)-3-(1-hydroxypropan-2-yl)-8-(pyridin-3-yl)-6-(5-(trifluoromethyl)pyridin-2-yl)pyrido[3,4-d]pyrimidin-4(3H)-one;
(xlvii) (S)-8-(3-fluorophenyl)-3-(1-hydroxypropan-2-yl)-6-(5-(trifluoromethyl)pyridin-2-yl)pyrido[3,4-d]pyrimidin-4(3H)-one;
(xlviii) (S)-8-(3-fluorophenyl)-3-(1-hydroxypropan-2-yl)-6-(p-tolyl)pyrido[3,4-d]pyrimidin-4(3H)-one;
(xlix) (S)-8-(3-fluorophenyl)-3-(1-hydroxypropan-2-yl)-6-(4-(trifluoromethoxy)phenyl)pyrido[3,4-d]pyrimidin-4(3H)-one;
(1) (S)-3-(1-hydroxypropan-2-yl)-8-(pyridin-3-yl)-6-(6-(trifluoromethoxy)pyridin-3-yl)pyrido[3,4-d]pyrimidin-4(3H)-one
(li) (S)-3-(2-hydroxy-2-methylpropyl)-8-(1H-pyrazol-4-yl)-6-(6-(trifluoromethyl)pyridin-3-yl)pyrido[3,4-d]pyrimidin-4(3H)-one
(lii)methyl (S)-5-(3-(1-hydroxypropan-2-yl)-4-oxo-8-(pyridin-3-yl)-3,4-dihydropyrido[3,4-d]pyrimidin-6-yl)picolinate
(liii) (S)-3-(1-hydroxypropan-2-yl)-6-(isothiazol-4-yl)-8-(pyridin-3-yl)pyrido[3,4-d]pyrimidin-4(3H)-one
(liv) 3-(2-hydroxy-2-methylpropyl)-8-(isothiazol-4-yl)-6-(6-(trifluoromethyl)pyridin-3-yl)pyrido[3,4-d]pyrimidin-4(3H)-one
(lv)(S)-3-(1-hydroxypropan-2-yl)-8-(isothiazol-4-yl)-6-(6-(trifluoromethyl)pyridin-3-yl)pyrido[3,4-d]pyrimidin-4(3H)-one
(lvi) (S)-3-(1-hydroxypropan-2-yl)-6,8-di(isothiazol-4-yl)pyrido[3,4-d]pyrimidin-4(3H)-one
(lvii) (S)-8-(pyridin-3-yl)-3-(3,3,3-trifluoro-2-hydroxypropyl)-6-(6-(trifluoromethyl)pyridin-3-yl)pyrido[3,4-d]pyrimidin-4(3H)-one
(lviii) (S)-6-(4-chlorophenyl)-3-(1-hydroxypropan-2-yl)-8-(isothiazol-4-yl)pyrido[3,4-d]pyrimidin-4(3H)-one
(lix) 3-(2-hydroxy-2-methylpropyl)-6,8-di(pyridin-3-yl)pyrido[3,4-d]pyrimidin-4(3H)-one
(lx) 3-(2-hydroxy-2-methylpropyl)-8-(1-methyl-1H-pyrazol-4-yl)-6-(6-(trifluoromethyl)pyridin-3-yl)pyrido[3,4-d]pyrimidin-4(3H)-one
(lxi) 6-(4-chloro-2-methylphenyl)-3-(2-hydroxy-2-methylpropyl)-8-(1-methyl-1H-pyrazol-4-yl)pyrido[3,4-d]pyrimidin-4(3H)-one
(lxii) (S)-3-(1-hydroxypropan-2-yl)-8-(1H-pyrazol-4-yl)-6-(6-(trifluoromethyl)pyridin-3-yl)pyrido[3,4-d]pyrimidin-4(3H)-one
(lxiii) (S)-3-(1-hydroxypropan-2-yl)-8-(2-methylpyridin-3-yl)-6-(6-(trifluoromethyl)pyridin-3-yl)pyrido[3,4-d]pyrimidin-4(3H)-one
(lxiv) (S)-3-(1-hydroxypropan-2-yl)-8-(4-methylpyridin-3-yl)-6-(6-(trifluoromethyl)pyridin-3-yl)pyrido[3,4-d]pyrimidin-4(3H)-one
(lxv) (S)-3-(1-hydroxypropan-2-yl)-6-(4-methylthiazol-5-yl)-8-(pyridin-3-yl)pyrido[3,4-d]pyrimidin-4(3H)-one
(lxvi) (S)-6-(2-cyclopropylthiazol-5-yl)-3-(1-hydroxypropan-2-yl)-8-(pyridin-3-yl)pyrido[3,4-d]pyrimidin-4(3H)-one
(lxvii) (S)-3-(1-hydroxypropan-2-yl)-6-(2-isopropylthiazol-5-yl)-8-(pyridin-3-yl)pyrido[3,4-d]pyrimidin-4(3H)-one
(lxviii) (S)-3-(1-hydroxypropan-2-yl)-8-(1-methyl-1H-pyrazol-4-yl)-6-(6-(trifluoromethyl)pyridin-3-yl)pyrido[3,4-d]pyrimidin-4(3H)-one
(lxix) (S)-3-(1-hydroxypropan-2-yl)-6,8-bis(6-(trifluoromethyl)pyridin-3-yl)pyrido[3,4-d]pyrimidin-4(3H)-one
(lxx) 6-(4-chlorophenyl)-3-((3S,4S)-4-hydroxytetrahydrofuran-3-yl)-8-(pyridin-3-yl)pyrido[3,4-d]pyrimidin-4(3H)-one.
(lxxi) 6-(4-chlorophenyl)-3-((3R,4R)-4-hydroxytetrahydrofuran-3-yl)-8-(pyridin-3-yl)pyrido[3,4-d]pyrimidin-4(3H)-one
(lxxii) 3-(2-hydroxyethyl)-8-(pyridin-3-yl)-6-(4-(trifluoromethoxy)phenyl)pyrido[3,4-d]pyrimidin-4(3H)-one
(lxxiii) (S)-6-(4-chlorophenyl)-3-(1-hydroxypropan-2-yl)-8-(1-methyl-6-oxo-1,6-dihydropyridin-3-yl)pyrido[3,4-d]pyrimidin-4(3H)-one
(lxxiv) (S)-6-(6-cyclopropylpyridin-3-yl)-3-(1-hydroxypropan-2-yl)-8-(pyridin-3-yl)pyrido[3,4-d]pyrimidin-4(3H)-one
(lxxv) (S)-3-(1-hydroxypropan-2-yl)-6-(4-methyl-6-(trifluoromethyl)pyridin-3-yl)-8-(pyridin-3-yl)pyrido[3,4-d]pyrimidin-4(3H)-one
(lxxvi) (S)-3-(1-hydroxypropan-2-yl)-8-(1-methyl-1H-pyrazol-4-yl)-6-(pyridin-3-yl)pyrido[3,4-d]pyrimidin-4(3H)-one
(lxxvii) (S)-6-(cyclohex-1-en-1-yl)-3-(1-hydroxypropan-2-yl)-8-(pyridin-3-yl)pyrido[3,4-d]pyrimidin-4(3H)-one
(lxxviii) (S)-6,8-bis(5-fluoropyridin-3-yl)-3-(1-hydroxypropan-2-yl)pyrido[3,4-d]pyrimidin-4(3H)-one
(lxxix) 3-((3R,4S)-4-hydroxytetrahydrofuran-3-yl)-8-(pyridin-3-yl)-6-(6-(trifluoromethyl)pyridin-3-yl)pyrido[3,4-d]pyrimidin-4(3H)-one
(lxxx) 3-(2-hydroxy-2-methylpropyl)-8-(pyridin-3-yl)-6-(2-(trifluoromethyl)pyridin-3-yl)pyrido[3,4-d]pyrimidin-4(3H)-one
(lxxxi) 6-(6-cyclopropylpyridin-3-yl)-3-(2-hydroxy-2-methylpropyl)-8-(pyridin-3-yl)pyrido[3,4-d]pyrimidin-4(3H)-one
(lxxxii) 6-(6-cyclopropylpyridin-3-yl)-3-(2-hydroxy-2-methylpropyl)-8-(1-methyl-1H-pyrazol-4-yl)pyrido[3,4-d]pyrimidin-4(3H)-one
(lxxxiii) (S)-6-(6-cyclopropylpyridin-3-yl)-3-(1-hydroxypropan-2-yl)-8-(1-methyl-1H-pyrazol-4-yl)pyrido[3,4-d]pyrimidin-4(3H)-one
(lxxxiv) (S)-3-(1-hydroxypropan-2-yl)-8-(pyridin-3-yl)-6-(thiazol-5-yl)pyrido[3,4-d]pyrimidin-4(3H)-one
(lxxxv) (S)-3-(1-hydroxypropan-2-yl)-8-(pyridin-3-yl)-6-(2-(trifluoromethyl)thiazol-5-yl)pyrido[3,4-d]pyrimidin-4(3H)-one
(lxxxvi) (S)-3-(1-hydroxypropan-2-yl)-8-(pyridin-3-yl)-6-(2-(trifluoromethyl)pyrimidin-5-yl)pyrido[3,4-d]pyrimidin-4(3H)-one
(lxxxvii) 3-(2-hydroxy-2-methylpropyl)-8-(pyridin-3-yl)-6-(2-(trifluoromethyl)thiazol-5-yl)pyrido[3,4-d]pyrimidin-4(3H)-one
(lxxxviii) 3-((3S,4S)-4-hydroxytetrahydrofuran-3-yl)-8-(1-methyl-1H-pyrazol-4-yl)-6-(6-(trifluoromethyl)pyridin-3-yl)pyrido[3,4-d]pyrimidin-4(3H)-one
(lxxxix) (S)-3-(1-hydroxypropan-2-yl)-6-(2-methylthiazol-5-yl)-8-(pyridin-3-yl)pyrido[3,4-d]pyrimidin-4(3H)-one
(xc) (S)-6-(4-chlorophenyl)-8-(3-fluorophenyl)-3-(1-hydroxy-3-methylbutan-2-yl)pyrido[3,4-d]pyrimidin-4(3H)-one
(xci) (S)-3-(1-hydroxypropan-2-yl)-6-(piperidin-1-yl)-8-(pyridin-3-yl)pyrido[3,4-d]pyrimidin-4(3H)-one
(xcii) 3-(2-hydroxy-2-methylpropyl)-8-(isothiazol-4-yl)-6-(5-(trifluoromethyl)pyridin-2-yl)pyrido[3,4-d]pyrimidin-4(3H)-one
(xciii) (S)-3-(1-hydroxypropan-2-yl)-8-(isothiazol-4-yl)-6-(5-(trifluoromethyl)pyridin-2-yl)pyrido[3,4-d]pyrimidin-4(3H)-one
(xciv) 3-(2-hydroxy-2-methylpropyl)-8-(1-methyl-1H-pyrazol-4-yl)-6-(5-(trifluoromethyl)pyridin-2-yl)pyrido[3,4-d]pyrimidin-4(3H)-one
(xcv) (S)-8-(pyridin-3-yl)-3-(3,3,3-trifluoro-2-hydroxypropyl)-6-(5-(trifluoromethyl)pyridin-2-yl)pyrido[3,4-d]pyrimidin-4(3H)-one
(xcvi) (3-(2-hydroxy-2-methylpropyl)-8-(pyridin-3-yl)-6-(5-(trifluoromethyl)pyridin-2-yl)pyrido[3,4-d]pyrimidin-4(3H)-one
(xcvii) 3-(1,1-dioxidotetrahydrothiophen-3-yl)-8-(pyridin-3-yl)-6-(6-(trifluoromethyl)pyridin-3-yl)pyrido[3,4-d]pyrimidin-4(3H)-one
(xcviii) (R)-3-(1,1-dioxidotetrahydrothiophen-3-yl)-8-(pyridin-3-yl)-6-(6-(trifluoromethyl)pyridin-3-yl)pyrido[3,4-d]pyrimidin-4(3H)-one
(xcix) (R)-3-(2-hydroxypropyl)-8-(1-methyl-1H-pyrazol-4-yl)-6-(6-(trifluoromethyl)pyridin-3-yl)pyrido[3,4-d]pyrimidin-4(3H)-one
(c) (S)-3-(2-hydroxypropyl)-8-(1-methyl-1H-pyrazol-4-yl)-6-(6-(trifluoromethyl)pyridin-3-yl)pyrido[3,4-d]pyrimidin-4(3H)-one
(ci)(R)-8-(1-methyl-1H-pyrazol-4-yl)-3-(3,3,3-trifluoro-2-hydroxypropyl)-6-(6-(trifluoromethyl)pyridin-3-yl)pyrido[3,4-d]pyrimidin-4(3H)-one
(cii) (S)-8-(1-methyl-1H-pyrazol-4-yl)-3-(3,3,3-trifluoro-2-hydroxypropyl)-6-(6-(trifluoromethyl)pyridin-3-yl)pyrido[3,4-d]pyrimidin-4(3H)-one
(ciii) (R)-3-(2-hydroxypropyl)-8-(pyridin-3-yl)-6-(6-(trifluoromethyl)pyridin-3-yl)pyrido[3,4-d]pyrimidin-4(3H)-one
(civ) (R)-8-(1-methyl-1H-pyrazol-4-yl)-3-(3,3,3-trifluoro-2-hydroxypropyl)-6-(5-(trifluoromethyl)pyridin-2-yl)pyrido[3,4-d]pyrimidin-4(3H)-one
(cv) 3-((3S,4R)-4-hydroxytetrahydrofuran-3-yl)-8-(1-methyl-1H-pyrazol-4-yl)-6-(6-(trifluoromethyl)pyridin-3-yl)pyrido[3,4-d]pyrimidin-4(3H)-one
(cvi) 3-((3R,4S)-4-hydroxytetrahydrofuran-3-yl)-8-(1-methyl-1H-pyrazol-4-yl)-6-(6-(trifluoromethyl)pyridin-3-yl)pyrido[3,4-d]pyrimidin-4(3H)-one
(cvii) 6-(4-chlorophenyl)-3-((3S,4R)-4-hydroxytetrahydrofuran-3-yl)-8-(1-methyl-1H-pyrazol-4-yl)pyrido[3,4-d]pyrimidin-4(3H)-one
(cviii) 3-((3S,4R)-4-hydroxytetrahydrofuran-3-yl)-8-(pyridin-3-yl)-6-(6-(trifluoromethyl)pyridin-3-yl)pyrido[3,4-d]pyrimidin-4(3H)-one
(cix) (S)-6-(6-cyclopropylpyridin-3-yl)-8-(pyridin-3-yl)-3-(3,3,3-trifluoro-2-hydroxypropyl)pyrido[3,4-d]pyrimidin-4(3H)-one
(cx) (R)-6-(6-cyclopropylpyridin-3-yl)-8-(pyridin-3-yl)-3-(3,3,3-trifluoro-2-hydroxypropyl)pyrido[3,4-d]pyrimidin-4(3H)-one
(cxi) (R)-8-(pyridin-3-yl)-3-(3,3,3-trifluoro-2-hydroxypropyl)-6-(6-(trifluoromethyl)pyridin-3-yl)pyrido[3,4-d]pyrimidin-4(3H)-one
(cxii) (R)-8-(pyridin-3-yl)-3-(3,3,3-trifluoro-2-hydroxypropyl)-6-(4-(trifluoromethoxy)phenyl)pyrido[3,4-d]pyrimidin-4(3H)-one
(cxiii) (S)-8-(pyridin-3-yl)-3-(3,3,3-trifluoro-2-hydroxypropyl)-6-(4-(trifluoromethoxy)phenyl)pyrido[3,4-d]pyrimidin-4(3H)-one
(cxiv) 6-(4-chlorophenyl)-3-((3S,4R)-4-hydroxytetrahydrofuran-3-yl)-8-(pyridin-3-yl)pyrido[3,4-d]pyrimidin-4(3H)-one
(cxv) 3-((3S,4S)-4-hydroxytetrahydrofuran-3-yl)-8-(pyridin-3-yl)-6-(6-(trifluoromethyl)pyridin-3-yl)pyrido[3,4-d]pyrimidin-4(3H)-one
(cxvi) methyl (S)-2-(4-oxo-8-(pyridin-3-yl)-6-(6-(trifluoromethyl)pyridin-3-yl)pyrido[3,4-d]pyrimidin-3(4H)-yl)propanoate
(cxvii) 6-(4-chlorophenyl)-3-(4-hydroxy-1-methylpyrrolidin-3-yl)-8-(pyridin-3-yl)pyrido[3,4-d]pyrimidin-4(3H)-one
(cxviii) 6-(4-chlorophenyl)-3-((3R,4R)-4-hydroxypyrrolidin-3-yl)-8-(pyridin-3-yl)pyrido[3,4-d]pyrimidin-4(3H)-one
(cxix) (R)-6-(6-cyclopropylpyridin-3-yl)-8-(l-methyl-1H-pyrazol-4-yl)-3-(3,3,3-trifluoro-2-hydroxypropyl)pyrido[3,4-d]pyrimidin-4(3H)-one
(cxx) (S)-6-(6-cyclopropylpyridin-3-yl)-8-(l-methyl-1H-pyrazol-4-yl)-3-(3,3,3-trifluoro-2-hydroxypropyl)pyrido[3,4-d]pyrimidin-4(3H)-one
(cxxi) 3-((3S,4R)-4-hydroxytetrahydrofuran-3-yl)-8-(l-methyl-1H-pyrazol-4-yl)-6-(5-(trifluoromethyl)pyridin-2-yl)pyrido[3,4-d]pyrimidin-4(3H)-one
(cxxii) 3-((3R,4S)-4-hydroxytetrahydrofuran-3-yl)-8-(l-methyl-1H-pyrazol-4-yl)-6-(5-(trifluoromethyl)pyridin-2-yl)pyrido[3,4-d]pyrimidin-4(3H)-one
(cxxiii) 3-((3S,4R)-4-hydroxytetrahydrofuran-3-yl)-8-(pyridin-3-yl)-6-(5-(trifluoromethyl)pyridin-2-yl)pyrido[3,4-d]pyrimidin-4(3H)-one
(cxxiv) 3-((3R,4S)-4-hydroxytetrahydrofuran-3-yl)-8-(pyridin-3-yl)-6-(5-(trifluoromethyl)pyridin-2-yl)pyrido[3,4-d]pyrimidin-4(3H)-one
(cxxv) (R)-3-(2-hydroxypropyl)-8-(1-methyl-1H-pyrazol-4-yl)-6-(5-(trifluoromethyl)pyridin-2-yl)pyrido[3,4-d]pyrimidin-4(3H)-one
(cxxvi) (S)-3-(2-hydroxypropyl)-8-(i-methyl-1H-pyrazol-4-yl)-6-(5-(trifluoromethyl)pyridin-2-yl)pyrido[3,4-d]pyrimidin-4(3H)-one
(cxxvii) (S)-3-(2-hydroxypropyl)-8-(pyridin-3-yl)-6-(5-(trifluoromethyl)pyridin-2-yl)pyrido[3,4-d]pyrimidin-4(3H)-one
(cxxviii) (R)-3-(2-hydroxypropyl)-8-(pyridin-3-yl)-6-(5-(trifluoromethyl)pyridin-2-yl)pyrido[3,4-d]pyrimidin-4(3H)-one
(cxxix) 3-((3S,4S)-4-hydroxytetrahydrofuran-3-yl)-8-(pyridin-3-yl)-6-(5-(trifluoromethyl)pyridin-2-yl)pyrido[3,4-d]pyrimidin-4(3H)-one
(cxxx) (S)-3-(2-hydroxypropyl)-8-(pyridin-3-yl)-6-(6-(trifluoromethyl)pyridin-3-yl)pyrido[3,4-d]pyrimidin-4(3H)-one
(cxxxi) (S)-8-(1-methyl-1H-pyrazol-4-yl)-3-(3,3,3-trifluoro-2-hydroxypropyl)-6-(5-(trifluoromethyl)pyridin-2-yl)pyrido[3,4-d]pyrimidin-4(3H)-one
(cxxxii) (S)-3-(1-hydroxypropan-2-yl)-8-(1-methyl-1H-pyrazol-4-yl)-6-(5-(trifluoromethyl)pyridin-2-yl)pyrido[3,4-d]pyrimidin-4(3H)-one
(cxxxiii) 3-((3S,4S)-4-hydroxytetrahydrofuran-3-yl)-8-(1-methyl-1H-pyrazol-4-yl)-6-(5-(trifluoromethyl)pyridin-2-yl)pyrido[3,4-d]pyrimidin-4(3H)-one
(cxxxiv) 6-(4-chlorophenyl)-3-((3R,4S)-4-hydroxytetrahydrofuran-3-yl)-8-(pyridin-3-yl)pyrido[3,4-d]pyrimidin-4(3H)-one
(cxxxv) 6-(4-Chlorophenyl)-3-((3R,4S)-4-hydroxytetrahydrofuran-3-yl)-8-(1-methyl-1H-pyrazol-4-yl)pyrido[3,4-d]pyrimidin-4(3H)-one
(cxxxvi) (R)-8-(pyridin-3-yl)-3-(3,3,3-trifluoro-2-hydroxypropyl)-6-(5-(trifluoromethyl)pyridin-2-yl)pyrido[3,4-d]pyrimidin-4(3H)-one
(cxxxvii) (S)-2-(4-oxo-8-(pyridin-3-yl)-6-(6-(trifluoromethyl)pyridin-3-yl)pyrido[3,4-d]pyrimidin-3(4H)-yl)propanoic acid
(cxxxviii) (S)-N-methyl-2-(4-oxo-8-(pyridin-3-yl)-6-(6-(trifluoromethyl)pyridin-3-yl)pyrido[3,4-d]pyrimidin-3(4H)-yl)propanamide
(cxxxix) (S)-N,N-dimethyl-2-(4-oxo-8-(pyridin-3-yl)-6-(6-(trifluoromethyl)pyridin-3-yl)pyrido[3,4-d]pyrimidin-3(4H)-yl)propenamide
(cxl) 3-(2-hydroxy-2-methylpropyl)-8-(1H-pyrazol-4-yl)-6-(5-(trifluoromethyl)pyridin-2-yl)pyrido[3,4-d]pyrimidin-4(3H)-one
(cxli) 3-(2-hydroxy-2-methylpropyl)-8-(1H-imidazol-1-yl)-6-(5-(trifluoromethyl)pyridin-2-yl)pyrido[3,4-d]pyrimidin-4(3H)-one
(cxlii) 3-(2-hydroxy-2-methylpropyl)-8-(1H-imidazol-1-yl)-6-(6-(trifluoromethyl)pyridin-2-yl)pyrido[3,4-d]pyrimidin-4(3H)-one
(cxliii) (S)-3-(1-hydroxypropan-2-yl)-8-(1H-imidazol-1-yl)-6-(5-(trifluoromethyl)pyridin-2-yl)pyrido[3,4-d]pyrimidin-4(3H)-one
(cxliv) (S)-3-(1-hydroxypropan-2-yl)-8-(1H-imidazol-1-yl)-6-(6-(trifluoromethyl)pyridin-3-yl)pyrido[3,4-d]pyrimidin-4(3H)-one
(cxlv) (S)-3-(1-hydroxypropan-2-yl)-8-(1H-1,2,4-triazol-1-yl)-6-(6-(trifluoromethyl)pyridin-3-yl)pyrido[3,4-d]pyrimidin-4(3H)-one
(cxlvi) (S)-3-(1-hydroxypropan-2-yl)-8-(1H-pyrazol-1-yl)-6-(6-(trifluoromethyl)pyridin-3-yl)pyrido[3,4-d]pyrimidin-4(3H)-one
(cxlvii) (S)-3-(1-hydroxypropan-2-yl)-8-(1-methyl-1H-pyrazol-4-yl)-6-(2-(trifluoromethyl)thiazol-5-yl)pyrido[3,4-d]pyrimidin-4(3H)-one
(cxlviii) (S)-8-(diethylamino)-3-(1-hydroxypropan-2-yl)-6-(6-(trifluoromethyl)pyridin-3-yl)pyrido[3,4-d]pyrimidin-4(3H)-one
(cxlix) (S)-3-(1-hydroxypropan-2-yl)-8-(piperidin-1-yl)-6-(6-(trifluoromethyl)pyridin-3-yl)pyrido[3,4-d]pyrimidin-4(3H)-one
(cl)(S)-3-(1-hydroxypropan-2-yl)-8-(pyrrolidin-1-yl)-6-(6-(trifluoromethyl)pyridin-3-yl)pyrido[3,4-d]pyrimidin-4(3H)-one
(cli) (S)-6-(4-chlorophenyl)-3-(1-hydroxypropan-2-yl)-8-(piperidin-1-yl)pyrido[3,4-d]pyrimidin-4(3H)-one
(clii) (S)-3-(1-hydroxypropan-2-yl)-8-(pyridin-2-yl)-6-(6-(trifluoromethyl)pyridin-3-yl)pyrido[3,4-d]pyrimidin-4(3H)-one
(cliii) (S)-6-cyclohexyl-3-(1-hydroxypropan-2-yl)-8-(pyridin-3-yl)pyrido[3,4-d]pyrimidin-4(3H)-one
(cliv) (S)-3-(1-hydroxypropan-2-yl)-6-(pyridin-2-yl)-8-(pyridin-3-yl)pyrido[3,4-d]pyrimidin-4(3H)-one
(clv) (S)-3-(1-hydroxypropan-2-yl)-6-(2-methylthiazol-4-yl)-8-(pyridin-3-yl)pyrido[3,4-d]pyrimidin-4(3H)-one
(clvi) (S)-3-(1-hydroxypropan-2-yl)-6-(1-methyl-1H-1,2,3-triazol-5-yl)-8-(pyridin-3-yl)pyrido[3,4-d]pyrimidin-4(3H)-one
(clvii) (R)-6-(4-chlorophenyl)-8-(pyridin-3-yl)-3-(3,3,3-trifluoro-2-hydroxypropyl)pyrido[3,4-d]pyrimidin-4(3H)-one
(clviii) (S)-6-(4-chlorophenyl)-8-(pyridin-3-yl)-3-(3,3,3-trifluoro-2-hydroxypropyl)pyrido[3,4-d]pyrimidin-4(3H)-one
(clix) (S)-3-(1-hydroxypropan-2-yl)-8-(1-methyl-1,2,5,6-tetrahydropyridin-3-yl)-6-(6-(trifluoromethyl)pyridin-3-yl)pyrido[3,4-d]pyrimidin-4(3H)-one
(clx) 6-(4-chlorophenyl)-3-((3S,4S)-4-hydroxytetrahydrofuran-3-yl)-8-(1-methyl-1H-pyrazol-4-yl)pyrido[3,4-d]pyrimidin-4(3H)-one
(clxi) (S)-3-(1-hydroxypropan-2-yl)-6-(2-methylpyrimidin-5-yl)-8-(pyridin-3-yl)pyrido[3,4-d]pyrimidin-4(3H)-one
(clxii) 3-(2-hydroxy-2-methylpropyl)-8-(1-(trifluoromethyl)-1H-pyrazol-4-yl)-6-(6-(trifluoromethyl)pyridin-3-yl)pyrido[3,4-d]pyrimidin-4(3H)-one
(clxiii) (S)-3-(1-hydroxypropan-2-yl)-8-(1-methyl-1H-pyrazol-4-yl)-6-(2-(trifluoromethyl)pyrimidin-5-yl)pyrido[3,4-d]pyrimidin-4(3H)-one
(clxiv) 3-(2-hydroxy-2-methylpropyl)-8-(pyridin-3-yl)-6-(2-(trifluoromethyl)pyrimidin-5-yl)pyrido[3,4-d]pyrimidin-4(3H)-one
(clxv) (S)-5-(3-(1-hydroxypropan-2-yl)-4-oxo-8-(pyridin-3-yl)-3,4-dihydropyrido[3,4-d]pyrimidin-6-yl)picolinic acid
(clxvi) (S)-3-(1-hydroxypropan-2-yl)-6-(6-methylpyridin-3-yl)-8-(pyridin-3-yl)pyrido[3,4-d]pyrimidin-4(3H)-one
(clxvii) 3-(2-hydroxy-2-methylpropyl)-8-(1-methyl-1H-pyrazol-4-yl)-6-(2-(trifluoromethyl)pyrimidin-5-yl)pyrido[3,4-d]pyrimidin-4(3H)-one
(clxviii) 3,8-di(pyridin-3-yl)-6-(6-(trifluoromethyl)pyridin-3-yl)pyrido[3,4-d]pyrimidin-4(3H)-one
(clxix) 8-(pyridin-3-yl)-6-(6-(trifluoromethyl)pyridin-3-yl)pyrido[3,4-d]pyrimidin-4(3H)-one
(clxx) 3-((3R,4R)-4-hydroxytetrahydrofuran-3-yl)-8-(pyridin-3-yl)-6-(6-(trifluoromethyl)pyridin-3-yl)pyrido[3,4-d]pyrimidin-4(3H)-one
(clxxi) 3-((3R,4R)-4-hydroxytetrahydrofuran-3-yl)-8-(l-methyl-1H-pyrazol-4-yl)-6-(6-(trifluoromethyl)pyridin-3-yl)pyrido[3,4-d]pyrimidin-4(3H)-one
(clxxii) 6-(4-chlorophenyl)-3-((3R,4R)-4-hydroxytetrahydrofuran-3-yl)-8-(l-methyl-1H-pyrazol-4-yl)pyrido[3,4-d]pyrimidin-4(3H)-one
(clxxiii) 3-cyclopentyl-8-(pyridin-3-yl)-6-(6-(trifluoromethyl)pyridin-3-yl)pyrido[3,4-d]pyrimidin-4(3H)-one
(clxxiv) 3-phenyl-8-(pyridin-3-yl)-6-(6-(trifluoromethyl)pyridin-3-yl)pyrido[3,4-d]pyrimidin-4(3H)-one
(clxxv) 3-((3R,4R)-4-hydroxytetrahydrofuran-3-yl)-8-(pyridin-3-yl)-6-(5-(trifluoromethyl)pyridin-2-yl)pyrido[3,4-d]pyrimidin-4(3H)-one
(clxxvi) 3-((3R,4R)-4-hydroxytetrahydrofuran-3-yl)-8-(l-methyl-1H-pyrazol-4-yl)-6-(5-(trifluoromethyl)pyridin-2-yl)pyrido[3,4-d]pyrimidin-4(3H)-one
(clxxvii) (S)-3-(1-hydroxypropan-2-yl)-8-(1-methyl-1H-pyrazol-4-yl)-6-(5-(trifluoromethyl)pyridin-2-yl)pyrido[3,4-d]pyrimidin-4(3H)-one
(clxxviii) (S)-N-((3R,4S)-4-hydroxytetrahydrofuran-3-yl)-2-(4-oxo-8-(pyridin-3-yl)-6-(6-(trifluoromethyl)pyridin-3-yl)pyrido[3,4-d]pyrimidin-3(4H)-yl)propanamide
(clxxix) 3-(2-hydroxy-2-methylpropyl)-8-(1H-pyrazol-4-yl)-6-(5-(trifluoromethyl)pyridin-2-yl)pyrido[3,4-d]pyrimidin-4(3H)-one
(clxxx) (S)-6-(4-chlorophenyl)-3-(1-hydroxypropan-2-yl)-8-(1H-pyrazol-1-yl)pyrido[3,4-d]pyrimidin-4(3H)-one
(clxxxi) (S)-3-(1-hydroxypropan-2-yl)-8-(1H-pyrazol-1-yl)-6-(5-(trifluoromethyl)pyridin-2-yl)pyrido[3,4-d]pyrimidin-4(3H)-one
(clxxxii) 3-((3S,4R)-4-hydroxytetrahydrofuran-3-yl)-8-(pyridin-3-yl)-6-(2-(trifluoromethyl)thiazol-5-yl)pyrido[3,4-d]pyrimidin-4(3H)-one
(clxxxiii) 3-(2-hydroxy-2-methylpropyl)-8-(1-methyl-1H-pyrazol-4-yl)-6-(2-(trifluoromethyl)thiazol-5-yl)pyrido[3,4-d]pyrimidin-4(3H)-one
(clxxxiv) 3-((3S,4R)-4-hydroxytetrahydrofuran-3-yl)-8-(1-methyl-1H-pyrazol-4-yl)-6-(2-(trifluoromethyl)thiazol-5-yl)pyrido[3,4-d]pyrimidin-4(3H)-one
(clxxxv) 3-((3R,4S)-4-hydroxytetrahydrofuran-3-yl)-8-(1-methyl-1H-pyrazol-4-yl)-6-(2-(trifluoromethyl)thiazol-5-yl)pyrido[3,4-d]pyrimidin-4(3H)-one
(clxxxvi) 3-((3R,4S)-4-hydroxytetrahydrofuran-3-yl)-8-(pyridin-3-yl)-6-(2-(trifluoromethyl)thiazol-5-yl)pyrido[3,4-d]pyrimidin-4(3H)-one
(clxxxvii) (S)-3-(1-hydroxypropan-2-yl)-8-morpholino-6-(6-(trifluoromethyl)pyridin-3-yl)pyrido[3,4-d]pyrimidin-4(3H)-one
(clxxxviii) 3-(2-hydroxy-2-methylpropyl)-8-(piperidin-1-yl)-6-(6-(trifluoromethyl)pyridin-3-yl)pyrido[3,4-d]pyrimidin-4(3H)-one
(clxxxix) (S)-3-(1-hydroxypropan-2-yl)-6-(5-methylpyridin-2-yl)-8-(pyridin-3-yl)pyrido[3,4-d]pyrimidin-4(3H)-one
(cxc) (S)-3-(1-hydroxypropan-2-yl)-6-(5-methylpyrimidin-2-yl)-8-(pyridin-3-yl)pyrido[3,4-d]pyrimidin-4(3H)-one
(cxci) (S)-8-(cyclohex-1-en-1-yl)-3-(1-hydroxypropan-2-yl)-6-(5-(trifluoromethyl)pyridin-2-yl)pyrido[3,4-d]pyrimidin-4(3H)-one
(cxcii) (S)-8-cyclohexyl-3-(1-hydroxypropan-2-yl)-6-(5-(trifluoromethyl)pyridin-2-yl)pyrido[3,4-d]pyrimidin-4(3H)-one
(cxciii) (S)-3-(1-hydroxypropan-2-yl)-N,N-dimethyl-4-oxo-8-(pyridin-3-yl)-3,4-dihydropyrido[3,4-d]pyrimidine-6-carboxamide
(cxciv) (S)-3-(1-hydroxypropan-2-yl)-8-(1H-pyrazol-4-yl)-6-(5-(trifluoromethyl)pyridin-2-yl)pyrido[3,4-d]pyrimidin-4(3H)-one
(cxcv) (S)-3-(1-hydroxypropan-2-yl)-6-(2-methoxyethyl)-8-(pyridin-3-yl)pyrido[3,4-d]pyrimidin-4(3H)-one
(cxcvi) (S)-3-(1-hydroxypropan-2-yl)-8-(2-methoxyethyl)-6-(5-(trifluoromethyl)pyridin-2-yl)pyrido[3,4-d]pyrimidin-4(3H)-one.
In some embodiments, an AhR antagonist is chosen from the compounds below and pharmaceutically acceptable salts thereof:
(i) (S)-8-(5-fluoropyridin-3-yl)-3-(1-hydroxypropan-2-yl)-6-(4-(trifluoromethoxy)phenyl)pyrido[3,4-d]pyrimidin-4(3H)-one;
(ii) (S)-6-(4-chlorophenyl)-3-(1-hydroxypropan-2-yl)-8-(6-oxo-1,6-dihydropyridin-3-yl)pyrido[3,4-d]pyrimidin-4(3H)-one;
(iii)(S)-8-(benzo[d][1,3]dioxol-4-yl)-6-(4-chlorophenyl)-3-(1-hydroxypropan-2-yl)pyrido[3,4-d]pyrimidin-4(3H)-one;
(iv)(S)-3-(1-hydroxypropan-2-yl)-8-(pyridin-4-yl)-6-(6-(trifluoromethyl)pyridin-3-yl)pyrido[3,4-d]pyrimidin-4(3H)-one;
(v) (S)-8-(5-fluoropyridin-3-yl)-3-(1-hydroxypropan-2-yl)-6-(4-(trifluoromethyl)phenyl)pyrido[3,4-d]pyrimidin-4(3H)-one;
(vi)(S)-3-(1-hydroxypropan-2-yl)-6,8-di(pyridin-4-yl)pyrido[3,4-d]pyrimidin-4(3H)-one;
(vii) (S)-6-(4-chlorophenyl)-8-(3-fluorophenyl)-3-(1-hydroxypropan-2-yl)pyrido[3,4-d]pyrimidin-4(3H)-one;
(viii) (S)-8-(3-fluorophenyl)-3-(1-hydroxypropan-2-yl)-6-(6-(trifluoromethyl)pyridin-3-yl)pyrido[3,4-d]pyrimidin-4(3H)-one;
(ix) 3-(2-hydroxy-2-methylpropyl)-8-(pyridin-3-yl)-6-(6-(trifluoromethyl)pyridin-3-yl)pyrido[3,4-d]pyrimidin-4(3H)-one;
(x) 6,8-di(pyridin-3-yl)-3-(3,3,3-trifluoro-2-hydroxypropyl)pyrido[3,4-d]pyrimidin-4(3H)-one;
(xi)(S)-6-chloro-3-(1-hydroxypropan-2-yl)-8-(pyridin-3-yl)pyrido[3,4-d]pyrimidin-4(3H)-one;
(xii) (S)-3-(1-hydroxypropan-2-yl)-8-(pyridin-3-yl)-6-(6-(trifluoromethyl)pyridin-3-yl)pyrido[3,4-d]pyrimidin-4(3H)-one;
(xiii) 6-(4-chlorophenyl)-8-(pyridin-3-yl)-3-(3,3,3-trifluoro-2-hydroxypropyl)pyrido[3,4-d]pyrimidin-4(3H)-one;
(xiv) 8-(pyridin-3-yl)-3-(3,3,3-trifluoro-2-hydroxypropyl)-6-(4-(trifluoromethoxy)phenyl)pyrido[3,4-d]pyrimidin-4(3H)-one;
(xv) 6-(4-chlorophenyl)-8-(1-methyl-1H-pyrazol-4-yl)-3-(3,3,3-trifluoro-2-hydroxypropyl)pyrido[3,4-d]pyrimidin-4(3H)-one;
(xvi) 3-(2-hydroxy-2-methylpropyl)-8-(pyridin-3-yl)-6-(4-(trifluoromethyl)phenyl)pyrido[3,4-d]pyrimidin-4(3H)-one;
(xvii) (S)-3-(1-hydroxypropan-2-yl)-8-(pyridin-3-yl)-6-(p-tolyl)pyrido[3,4-d]pyrimidin-4(3H)-one;
(xviii) 6-(4-chlorophenyl)-3-(2-hydroxy-2-methylpropyl)-8-(pyridin-3-yl)pyrido[3,4-d]pyrimidin-4(3H)-one;
(xix) 3-(2-hydroxy-2-methylpropyl)-6,8-bis(1-methyl-1H-pyrazol-4-yl)pyrido[3,4-d]pyrimidin-4(3H)-one;
(xx) (S)-3-(1-hydroxypropan-2-yl)-6,8-di(pyridin-3-yl)pyrido[3,4-d]pyrimidin-4(3H)-one;
(xxi) 6-(4-chlorophenyl)-3-(2-hydroxy-2-methylpropyl)-8-(1-methyl-1H-pyrazol-4-yl)pyrido[3,4-d]pyrimidin-4(3H)-one;
(xxii) (S)-3-(1-hydroxypropan-2-yl)-8-(pyridin-3-yl)-6-(4-(trifluoromethoxy)phenyl)pyrido[3,4-d]pyrimidin-4(3H)-one;
(xxiii) (S)-3-(1-hydroxypropan-2-yl)-8-(pyridin-3-yl)-6-(4-(trifluoromethyl)phenyl)pyrido[3,4-d]pyrimidin-4(3H)-one;
(xxiv) (S)-6-(4-chlorophenyl)-3-(1-hydroxypropan-2-yl)-8-(pyridin-3-yl)pyrido[3,4-d]pyrimidin-4(3H)-one;
(xxv) (S)-3-(1-hydroxypropan-2-yl)-8-(1-methyl-1H-pyrazol-4-yl)-6-(4-(trifluoromethoxy)phenyl)pyrido[3,4-d]pyrimidin-4(3H)-one;
(xxvi) (S)-3-(1-hydroxypropan-2-yl)-8-(1-methyl-1H-pyrazol-4-yl)-6-phenylpyrido[3,4-d]pyrimidin-4(3H)-one;
(xxvii) (S)-6-(4-chlorophenyl)-3-(1-hydroxypropan-2-yl)-8-(1-methyl-1H-pyrazol-4-yl)pyrido[3,4-d]pyrimidin-4(3H)-one;
(xxviii) 3-methyl-8-(pyridin-3-yl)-6-(4-(trifluoromethoxy)phenyl)pyrido[3,4-d]pyrimidin-4(3H)-one;
(xxix) Rac-6-(4-chlorophenyl)-3-((trans)-4-hydroxytetrahydrofuran-3-yl)-8-(pyridin-3-yl)pyrido[3,4-d]pyrimidin-4(3H)-one;
(xxx) (S)-6-(4-chlorophenyl)-3-(3-hydroxy-3-methylbutan-2-yl)-8-(1-methyl-1H-pyrazol-4-yl)pyrido[3,4-d]pyrimidin-4(3H)-one;
(xxxi) (R)-6-(4-chlorophenyl)-3-(3-hydroxy-3-methylbutan-2-yl)-8-(1-methyl-1H-pyrazol-4-yl)pyrido[3,4-d]pyrimidin-4(3H)-one;
(xxxii) rac-6-(4-chlorophenyl)-3-((cis)-4-hydroxytetrahydrofuran-3-yl)-8-(pyridin-3-yl)pyrido[3,4-d]pyrimidin-4(3H)-one;
(xxxiii) (R)-6-(4-chlorophenyl)-3-(3-hydroxy-3-methylbutan-2-yl)-8-(pyridin-3-yl)pyrido[3,4-d]pyrimidin-4(3H)-one;
(xxxiv) (S)-3-(3-hydroxy-3-methylbutan-2-yl)-6,8-di(pyridin-3-yl)pyrido[3,4-d]pyrimidin-4(3H)-one;
(xxxv) (S)-6,8-bis(3,5-difluorophenyl)-3-(1-hydroxy-3-methylbutan-2-yl)pyrido[3,4-d]pyrimidin-4(3H)-one;
(xxxvi) (S)-6-(4-chlorophenyl)-3-(3-hydroxy-3-methylbutan-2-yl)-8-(pyridin-3-yl)pyrido[3,4-d]pyrimidin-4(3H)-one;
(xxxvii) (S)-8-(3,5-difluorophenyl)-3-(1-hydroxy-3-methylbutan-2-yl)-6-(p-tolyl)pyrido[3,4-d]pyrimidin-4(3H)-one;
(xxxviii) 6-(4-chlorophenyl)-8-(3-fluorophenyl)-3-(2-hydroxy-2-methylpropyl)pyrido[3,4-d]pyrimidin-4(3H)-one;
(xxxix) (R)-6-(4-chlorophenyl)-8-(3-fluorophenyl)-3-(3-hydroxy-3-methylbutan-2-yl)pyrido[3,4-d]pyrimidin-4(3H)-one;
(xl)(S)-3-(1-(benzyloxy)propan-2-yl)-8-(3-fluorophenyl)-6-(p-tolyl)pyrido[3,4-d]pyrimidin-4(3H)-one;
(xli) (R)-6-(4-chlorophenyl)-8-(3-fluorophenyl)-3-(3,3,3-trifluoro-2-hydroxypropyl)pyrido[3,4-d]pyrimidin-4(3H)-one;
(xlii) (S)-6-(4-chlorophenyl)-8-(3-fluorophenyl)-3-(3,3,3-trifluoro-2-hydroxypropyl)pyrido[3,4-d]pyrimidin-4(3H)-one;
(xliii) (S)-3-(1-hydroxypropan-2-yl)-6-morpholino-8-(pyridin-3-yl)pyrido[3,4-d]pyrimidin-4(3H)-one;
(xliv) (S)-3-(1-hydroxypropan-2-yl)-8-(1H-imidazol-1-yl)-6-(4-(trifluoromethoxy)phenyl)pyrido[3,4-d]pyrimidin-4(3H)-one;
(xlv) (S)-3-(1-methoxypropan-2-yl)-8-(pyridin-3-yl)-6-(p-tolyl)pyrido[3,4-d]pyrimidin-4(3H)-one;
(xlvi) (S)-3-(1-hydroxypropan-2-yl)-8-(pyridin-3-yl)-6-(5-(trifluoromethyl)pyridin-2-yl)pyrido[3,4-d]pyrimidin-4(3H)-one;
(xlvii) (S)-8-(3-fluorophenyl)-3-(1-hydroxypropan-2-yl)-6-(5-(trifluoromethyl)pyridin-2-yl)pyrido[3,4-d]pyrimidin-4(3H)-one;
(xlviii) (S)-8-(3-fluorophenyl)-3-(1-hydroxypropan-2-yl)-6-(p-tolyl)pyrido[3,4-d]pyrimidin-4(3H)-one;
(xlix) (S)-8-(3-fluorophenyl)-3-(1-hydroxypropan-2-yl)-6-(4-(trifluoromethoxy)phenyl)pyrido[3,4-d]pyrimidin-4(3H)-one;
(1) (S)-3-(1-hydroxypropan-2-yl)-8-(pyridin-3-yl)-6-(6-(trifluoromethoxy)pyridin-3-yl)pyrido[3,4-d]pyrimidin-4(3H)-one
(li) (S)-3-(2-hydroxy-2-methylpropyl)-8-(1H-pyrazol-4-yl)-6-(6-(trifluoromethyl)pyridin-3-yl)pyrido[3,4-d]pyrimidin-4(3H)-one
(lii)methyl (S)-5-(3-(1-hydroxypropan-2-yl)-4-oxo-8-(pyridin-3-yl)-3,4-dihydropyrido[3,4-d]pyrimidin-6-yl)picolinate
(liii) (S)-3-(1-hydroxypropan-2-yl)-6-(isothiazol-4-yl)-8-(pyridin-3-yl)pyrido[3,4-d]pyrimidin-4(3H)-one
(liv) 3-(2-hydroxy-2-methylpropyl)-8-(isothiazol-4-yl)-6-(6-(trifluoromethyl)pyridin-3-yl)pyrido[3,4-d]pyrimidin-4(3H)-one
(lv)(S)-3-(1-hydroxypropan-2-yl)-8-(isothiazol-4-yl)-6-(6-(trifluoromethyl)pyridin-3-yl)pyrido[3,4-d]pyrimidin-4(3H)-one
(lvi) (S)-3-(1-hydroxypropan-2-yl)-6,8-di(isothiazol-4-yl)pyrido[3,4-d]pyrimidin-4(3H)-one
(lvii) (S)-8-(pyridin-3-yl)-3-(3,3,3-trifluoro-2-hydroxypropyl)-6-(6-(trifluoromethyl)pyridin-3-yl)pyrido[3,4-d]pyrimidin-4(3H)-one
(lviii) (S)-6-(4-chlorophenyl)-3-(1-hydroxypropan-2-yl)-8-(isothiazol-4-yl)pyrido[3,4-d]pyrimidin-4(3H)-one
(lix) 3-(2-hydroxy-2-methylpropyl)-6,8-di(pyridin-3-yl)pyrido[3,4-d]pyrimidin-4(3H)-one
(lx) 3-(2-hydroxy-2-methylpropyl)-8-(1-methyl-1H-pyrazol-4-yl)-6-(6-(trifluoromethyl)pyridin-3-yl)pyrido[3,4-d]pyrimidin-4(3H)-one
(lxi) 6-(4-chloro-2-methylphenyl)-3-(2-hydroxy-2-methylpropyl)-8-(1-methyl-1H-pyrazol-4-yl)pyrido[3,4-d]pyrimidin-4(3H)-one
(lxii) (S)-3-(1-hydroxypropan-2-yl)-8-(1H-pyrazol-4-yl)-6-(6-(trifluoromethyl)pyridin-3-yl)pyrido[3,4-d]pyrimidin-4(3H)-one
(lxiii) (S)-3-(1-hydroxypropan-2-yl)-8-(2-methylpyridin-3-yl)-6-(6-(trifluoromethyl)pyridin-3-yl)pyrido[3,4-d]pyrimidin-4(3H)-one
(lxiv) (S)-3-(1-hydroxypropan-2-yl)-8-(4-methylpyridin-3-yl)-6-(6-(trifluoromethyl)pyridin-3-yl)pyrido[3,4-d]pyrimidin-4(3H)-one
(lxv) (S)-3-(1-hydroxypropan-2-yl)-6-(4-methylthiazol-5-yl)-8-(pyridin-3-yl)pyrido[3,4-d]pyrimidin-4(3H)-one
(lxvi) (S)-6-(2-cyclopropylthiazol-5-yl)-3-(1-hydroxypropan-2-yl)-8-(pyridin-3-yl)pyrido[3,4-d]pyrimidin-4(3H)-one
(lxvii) (S)-3-(1-hydroxypropan-2-yl)-6-(2-isopropylthiazol-5-yl)-8-(pyridin-3-yl)pyrido[3,4-d]pyrimidin-4(3H)-one
(lxviii) (S)-3-(1-hydroxypropan-2-yl)-8-(1-methyl-1H-pyrazol-4-yl)-6-(6-(trifluoromethyl)pyridin-3-yl)pyrido[3,4-d]pyrimidin-4(3H)-one
(lxix) (S)-3-(1-hydroxypropan-2-yl)-6,8-bis(6-(trifluoromethyl)pyridin-3-yl)pyrido[3,4-d]pyrimidin-4(3H)-one
(lxx) 6-(4-chlorophenyl)-3-((3S,4S)-4-hydroxytetrahydrofuran-3-yl)-8-(pyridin-3-yl)pyrido[3,4-d]pyrimidin-4(3H)-one.
(lxxi) 6-(4-chlorophenyl)-3-((3R,4R)-4-hydroxytetrahydrofuran-3-yl)-8-(pyridin-3-yl)pyrido[3,4-d]pyrimidin-4(3H)-one
(lxxii) 3-(2-hydroxyethyl)-8-(pyridin-3-yl)-6-(4-(trifluoromethoxy)phenyl)pyrido[3,4-d]pyrimidin-4(3H)-one
(lxxiii) (S)-6-(4-chlorophenyl)-3-(1-hydroxypropan-2-yl)-8-(1-methyl-6-oxo-1,6-dihydropyridin-3-yl)pyrido[3,4-d]pyrimidin-4(3H)-one
(lxxiv) (S)-6-(6-cyclopropylpyridin-3-yl)-3-(1-hydroxypropan-2-yl)-8-(pyridin-3-yl)pyrido[3,4-d]pyrimidin-4(3H)-one
(lxxv) (S)-3-(1-hydroxypropan-2-yl)-6-(4-methyl-6-(trifluoromethyl)pyridin-3-yl)-8-(pyridin-3-yl)pyrido[3,4-d]pyrimidin-4(3H)-one
(lxxvi) (S)-3-(1-hydroxypropan-2-yl)-8-(1-methyl-1H-pyrazol-4-yl)-6-(pyridin-3-yl)pyrido[3,4-d]pyrimidin-4(3H)-one
(lxxvii) (S)-6-(cyclohex-1-en-1-yl)-3-(1-hydroxypropan-2-yl)-8-(pyridin-3-yl)pyrido[3,4-d]pyrimidin-4(3H)-one
(lxxviii) (S)-6,8-bis(5-fluoropyridin-3-yl)-3-(1-hydroxypropan-2-yl)pyrido[3,4-d]pyrimidin-4(3H)-one
(lxxix) 3-((3R,4S)-4-hydroxytetrahydrofuran-3-yl)-8-(pyridin-3-yl)-6-(6-(trifluoromethyl)pyridin-3-yl)pyrido[3,4-d]pyrimidin-4(3H)-one
(lxxx) 3-(2-hydroxy-2-methylpropyl)-8-(pyridin-3-yl)-6-(2-(trifluoromethyl)pyridin-3-yl)pyrido[3,4-d]pyrimidin-4(3H)-one
(lxxxi) 6-(6-cyclopropylpyridin-3-yl)-3-(2-hydroxy-2-methylpropyl)-8-(pyridin-3-yl)pyrido[3,4-d]pyrimidin-4(3H)-one
(lxxxii) 6-(6-cyclopropylpyridin-3-yl)-3-(2-hydroxy-2-methylpropyl)-8-(1-methyl-1H-pyrazol-4-yl)pyrido[3,4-d]pyrimidin-4(3H)-one
(lxxxiii) (S)-6-(6-cyclopropylpyridin-3-yl)-3-(1-hydroxypropan-2-yl)-8-(1-methyl-1H-pyrazol-4-yl)pyrido[3,4-d]pyrimidin-4(3H)-one
(lxxxiv) (S)-3-(1-hydroxypropan-2-yl)-8-(pyridin-3-yl)-6-(thiazol-5-yl)pyrido[3,4-d]pyrimidin-4(3H)-one
(lxxxv) (S)-3-(1-hydroxypropan-2-yl)-8-(pyridin-3-yl)-6-(2-(trifluoromethyl)thiazol-5-yl)pyrido[3,4-d]pyrimidin-4(3H)-one
(lxxxvi) (S)-3-(1-hydroxypropan-2-yl)-8-(pyridin-3-yl)-6-(2-(trifluoromethyl)pyrimidin-5-yl)pyrido[3,4-d]pyrimidin-4(3H)-one
(lxxxvii) 3-(2-hydroxy-2-methylpropyl)-8-(pyridin-3-yl)-6-(2-(trifluoromethyl)thiazol-5-yl)pyrido[3,4-d]pyrimidin-4(3H)-one
(lxxxviii) 3-((3S,4S)-4-hydroxytetrahydrofuran-3-yl)-8-(1-methyl-1H-pyrazol-4-yl)-6-(6-(trifluoromethyl)pyridin-3-yl)pyrido[3,4-d]pyrimidin-4(3H)-one
(lxxxix) (S)-3-(1-hydroxypropan-2-yl)-6-(2-methylthiazol-5-yl)-8-(pyridin-3-yl)pyrido[3,4-d]pyrimidin-4(3H)-one
(xc) (S)-6-(4-chlorophenyl)-8-(3-fluorophenyl)-3-(1-hydroxy-3-methylbutan-2-yl)pyrido[3,4-d]pyrimidin-4(3H)-one
(xci) (S)-3-(1-hydroxypropan-2-yl)-6-(piperidin-1-yl)-8-(pyridin-3-yl)pyrido[3,4-d]pyrimidin-4(3H)-one
(xcii) 3-(2-hydroxy-2-methylpropyl)-8-(isothiazol-4-yl)-6-(5-(trifluoromethyl)pyridin-2-yl)pyrido[3,4-d]pyrimidin-4(3H)-one
(xciii) (S)-3-(1-hydroxypropan-2-yl)-8-(isothiazol-4-yl)-6-(5-(trifluoromethyl)pyridin-2-yl)pyrido[3,4-d]pyrimidin-4(3H)-one
(xciv) 3-(2-hydroxy-2-methylpropyl)-8-(1-methyl-1H-pyrazol-4-yl)-6-(5-(trifluoromethyl)pyridin-2-yl)pyrido[3,4-d]pyrimidin-4(3H)-one
(xcv) (S)-8-(pyridin-3-yl)-3-(3,3,3-trifluoro-2-hydroxypropyl)-6-(5-(trifluoromethyl)pyridin-2-yl)pyrido[3,4-d]pyrimidin-4(3H)-one
(xcvi) (3-(2-hydroxy-2-methylpropyl)-8-(pyridin-3-yl)-6-(5-(trifluoromethyl)pyridin-2-yl)pyrido[3,4-d]pyrimidin-4(3H)-one
(xcvii) 3-(1,1-dioxidotetrahydrothiophen-3-yl)-8-(pyridin-3-yl)-6-(6-(trifluoromethyl)pyridin-3-yl)pyrido[3,4-d]pyrimidin-4(3H)-one
(xcviii) (R)-3-(1,1-dioxidotetrahydrothiophen-3-yl)-8-(pyridin-3-yl)-6-(6-(trifluoromethyl)pyridin-3-yl)pyrido[3,4-d]pyrimidin-4(3H)-one
(xcix) (R)-3-(2-hydroxypropyl)-8-(1-methyl-1H-pyrazol-4-yl)-6-(6-(trifluoromethyl)pyridin-3-yl)pyrido[3,4-d]pyrimidin-4(3H)-one
(c) (S)-3-(2-hydroxypropyl)-8-(1-methyl-1H-pyrazol-4-yl)-6-(6-(trifluoromethyl)pyridin-3-yl)pyrido[3,4-d]pyrimidin-4(3H)-one
(ci)(R)-8-(1-methyl-1H-pyrazol-4-yl)-3-(3,3,3-trifluoro-2-hydroxypropyl)-6-(6-(trifluoromethyl)pyridin-3-yl)pyrido[3,4-d]pyrimidin-4(3H)-one
(cii) (S)-8-(1-methyl-1H-pyrazol-4-yl)-3-(3,3,3-trifluoro-2-hydroxypropyl)-6-(6-(trifluoromethyl)pyridin-3-yl)pyrido[3,4-d]pyrimidin-4(3H)-one
(ciii) (R)-3-(2-hydroxypropyl)-8-(pyridin-3-yl)-6-(6-(trifluoromethyl)pyridin-3-yl)pyrido[3,4-d]pyrimidin-4(3H)-one
(civ) (R)-8-(1-methyl-1H-pyrazol-4-yl)-3-(3,3,3-trifluoro-2-hydroxypropyl)-6-(5-(trifluoromethyl)pyridin-2-yl)pyrido[3,4-d]pyrimidin-4(3H)-one
(cv) 3-((3S,4R)-4-hydroxytetrahydrofuran-3-yl)-8-(1-methyl-1H-pyrazol-4-yl)-6-(6-(trifluoromethyl)pyridin-3-yl)pyrido[3,4-d]pyrimidin-4(3H)-one
(cvi) 3-((3R,4S)-4-hydroxytetrahydrofuran-3-yl)-8-(1-methyl-1H-pyrazol-4-yl)-6-(6-(trifluoromethyl)pyridin-3-yl)pyrido[3,4-d]pyrimidin-4(3H)-one
(cvii) 6-(4-chlorophenyl)-3-((3S,4R)-4-hydroxytetrahydrofuran-3-yl)-8-(1-methyl-1H-pyrazol-4-yl)pyrido[3,4-d]pyrimidin-4(3H)-one
(cviii) 3-((3S,4R)-4-hydroxytetrahydrofuran-3-yl)-8-(pyridin-3-yl)-6-(6-(trifluoromethyl)pyridin-3-yl)pyrido[3,4-d]pyrimidin-4(3H)-one
(cix) (S)-6-(6-cyclopropylpyridin-3-yl)-8-(pyridin-3-yl)-3-(3,3,3-trifluoro-2-hydroxypropyl)pyrido[3,4-d]pyrimidin-4(3H)-one
(cx) (R)-6-(6-cyclopropylpyridin-3-yl)-8-(pyridin-3-yl)-3-(3,3,3-trifluoro-2-hydroxypropyl)pyrido[3,4-d]pyrimidin-4(3H)-one
(cxi) (R)-8-(pyridin-3-yl)-3-(3,3,3-trifluoro-2-hydroxypropyl)-6-(6-(trifluoromethyl)pyridin-3-yl)pyrido[3,4-d]pyrimidin-4(3H)-one
(cxii) (R)-8-(pyridin-3-yl)-3-(3,3,3-trifluoro-2-hydroxypropyl)-6-(4-(trifluoromethoxy)phenyl)pyrido[3,4-d]pyrimidin-4(3H)-one
(cxiii) (S)-8-(pyridin-3-yl)-3-(3,3,3-trifluoro-2-hydroxypropyl)-6-(4-(trifluoromethoxy)phenyl)pyrido[3,4-d]pyrimidin-4(3H)-one
(cxiv) 6-(4-chlorophenyl)-3-((3S,4R)-4-hydroxytetrahydrofuran-3-yl)-8-(pyridin-3-yl)pyrido[3,4-d]pyrimidin-4(3H)-one
(cxv) 3-((3S,4S)-4-hydroxytetrahydrofuran-3-yl)-8-(pyridin-3-yl)-6-(6-(trifluoromethyl)pyridin-3-yl)pyrido[3,4-d]pyrimidin-4(3H)-one
(cxvi) methyl (S)-2-(4-oxo-8-(pyridin-3-yl)-6-(6-(trifluoromethyl)pyridin-3-yl)pyrido[3,4-d]pyrimidin-3(4H)-yl)propanoate
(cxvii) 6-(4-chlorophenyl)-3-(4-hydroxy-1-methylpyrrolidin-3-yl)-8-(pyridin-3-yl)pyrido[3,4-d]pyrimidin-4(3H)-one
(cxviii) 6-(4-chlorophenyl)-3-((3R,4R)-4-hydroxypyrrolidin-3-yl)-8-(pyridin-3-yl)pyrido[3,4-d]pyrimidin-4(3H)-one
(cxix) (R)-6-(6-cyclopropylpyridin-3-yl)-8-(1-methyl-1H-pyrazol-4-yl)-3-(3,3,3-trifluoro-2-hydroxypropyl)pyrido[3,4-d]pyrimidin-4(3H)-one
(cxx) (S)-6-(6-cyclopropylpyridin-3-yl)-8-(1-methyl-1H-pyrazol-4-yl)-3-(3,3,3-trifluoro-2-hydroxypropyl)pyrido[3,4-d]pyrimidin-4(3H)-one
(cxxi) 3-((3S,4R)-4-hydroxytetrahydrofuran-3-yl)-8-(1-methyl-1H-pyrazol-4-yl)-6-(5-(trifluoromethyl)pyridin-2-yl)pyrido[3,4-d]pyrimidin-4(3H)-one
(cxxii) 3-((3R,4S)-4-hydroxytetrahydrofuran-3-yl)-8-(1-methyl-1H-pyrazol-4-yl)-6-(5-(trifluoromethyl)pyridin-2-yl)pyrido[3,4-d]pyrimidin-4(3H)-one
(cxxiii) 3-((3S,4R)-4-hydroxytetrahydrofuran-3-yl)-8-(pyridin-3-yl)-6-(5-(trifluoromethyl)pyridin-2-yl)pyrido[3,4-d]pyrimidin-4(3H)-one
(cxxiv) 3-((3R,4S)-4-hydroxytetrahydrofuran-3-yl)-8-(pyridin-3-yl)-6-(5-(trifluoromethyl)pyridin-2-yl)pyrido[3,4-d]pyrimidin-4(3H)-one
(cxxv) (R)-3-(2-hydroxypropyl)-8-(1-methyl-1H-pyrazol-4-yl)-6-(5-(trifluoromethyl)pyridin-2-yl)pyrido[3,4-d]pyrimidin-4(3H)-one
(cxxvi) (S)-3-(2-hydroxypropyl)-8-(1-methyl-1H-pyrazol-4-yl)-6-(5-(trifluoromethyl)pyridin-2-yl)pyrido[3,4-d]pyrimidin-4(3H)-one
(cxxvii) (S)-3-(2-hydroxypropyl)-8-(pyridin-3-yl)-6-(5-(trifluoromethyl)pyridin-2-yl)pyrido[3,4-d]pyrimidin-4(3H)-one
(cxxviii) (R)-3-(2-hydroxypropyl)-8-(pyridin-3-yl)-6-(5-(trifluoromethyl)pyridin-2-yl)pyrido[3,4-d]pyrimidin-4(3H)-one
(cxxix) 3-((3S,4S)-4-hydroxytetrahydrofuran-3-yl)-8-(pyridin-3-yl)-6-(5-(trifluoromethyl)pyridin-2-yl)pyrido[3,4-d]pyrimidin-4(3H)-one
(cxxx) (S)-3-(2-hydroxypropyl)-8-(pyridin-3-yl)-6-(6-(trifluoromethyl)pyridin-3-yl)pyrido[3,4-d]pyrimidin-4(3H)-one
(cxxxi) (S)-8-(1-methyl-1H-pyrazol-4-yl)-3-(3,3,3-trifluoro-2-hydroxypropyl)-6-(5-(trifluoromethyl)pyridin-2-yl)pyrido[3,4-d]pyrimidin-4(3H)-one
(cxxxii) (S)-3-(1-hydroxypropan-2-yl)-8-(1-methyl-1H-pyrazol-4-yl)-6-(5-(trifluoromethyl)pyridin-2-yl)pyrido[3,4-d]pyrimidin-4(3H)-one
(cxxxiii) 3-((3S,4S)-4-hydroxytetrahydrofuran-3-yl)-8-(1-methyl-1H-pyrazol-4-yl)-6-(5-(trifluoromethyl)pyridin-2-yl)pyrido[3,4-d]pyrimidin-4(3H)-one
(cxxxiv) 6-(4-chlorophenyl)-3-((3R,4S)-4-hydroxytetrahydrofuran-3-yl)-8-(pyridin-3-yl)pyrido[3,4-d]pyrimidin-4(3H)-one
(cxxxv) 6-(4-Chlorophenyl)-3-((3R,4S)-4-hydroxytetrahydrofuran-3-yl)-8-(1-methyl-1H-pyrazol-4-yl)pyrido[3,4-d]pyrimidin-4(3H)-one
(cxxxvi) (R)-8-(pyridin-3-yl)-3-(3,3,3-trifluoro-2-hydroxypropyl)-6-(5-(trifluoromethyl)pyridin-2-yl)pyrido[3,4-d]pyrimidin-4(3H)-one
(cxxxvii) (S)-2-(4-oxo-8-(pyridin-3-yl)-6-(6-(trifluoromethyl)pyridin-3-yl)pyrido[3,4-d]pyrimidin-3(4H)-yl)propanoic acid
(cxxxviii) (S)-N-methyl-2-(4-oxo-8-(pyridin-3-yl)-6-(6-(trifluoromethyl)pyridin-3-yl)pyrido[3,4-d]pyrimidin-3(4H)-yl)propanamide
(cxxxix) (S)-N,N-dimethyl-2-(4-oxo-8-(pyridin-3-yl)-6-(6-(trifluoromethyl)pyridin-3-yl)pyrido[3,4-d]pyrimidin-3(4H)-yl)propenamide
(cxl) 3-(2-hydroxy-2-methylpropyl)-8-(1H-pyrazol-4-yl)-6-(5-(trifluoromethyl)pyridin-2-yl)pyrido[3,4-d]pyrimidin-4(3H)-one
(cxli) 3-(2-hydroxy-2-methylpropyl)-8-(1H-imidazol-1-yl)-6-(5-(trifluoromethyl)pyridin-2-yl)pyrido[3,4-d]pyrimidin-4(3H)-one
(cxlii) 3-(2-hydroxy-2-methylpropyl)-8-(1H-imidazol-1-yl)-6-(6-(trifluoromethyl)pyridin-2-yl)pyrido[3,4-d]pyrimidin-4(3H)-one
(cxliii) (S)-3-(1-hydroxypropan-2-yl)-8-(1H-imidazol-1-yl)-6-(5-(trifluoromethyl)pyridin-2-yl)pyrido[3,4-d]pyrimidin-4(3H)-one
(cxliv) (S)-3-(1-hydroxypropan-2-yl)-8-(1H-imidazol-1-yl)-6-(6-(trifluoromethyl)pyridin-3-yl)pyrido[3,4-d]pyrimidin-4(3H)-one
(cxlv) (S)-3-(1-hydroxypropan-2-yl)-8-(1H-1,2,4-triazol-1-yl)-6-(6-(trifluoromethyl)pyridin-3-yl)pyrido[3,4-d]pyrimidin-4(3H)-one
(cxlvi) (S)-3-(1-hydroxypropan-2-yl)-8-(1H-pyrazol-1-yl)-6-(6-(trifluoromethyl)pyridin-3-yl)pyrido[3,4-d]pyrimidin-4(3H)-one
(cxlvii) (S)-3-(1-hydroxypropan-2-yl)-8-(1-methyl-1H-pyrazol-4-yl)-6-(2-(trifluoromethyl)thiazol-5-yl)pyrido[3,4-d]pyrimidin-4(3H)-one
(cxlviii) (S)-8-(diethylamino)-3-(1-hydroxypropan-2-yl)-6-(6-(trifluoromethyl)pyridin-3-yl)pyrido[3,4-d]pyrimidin-4(3H)-one
(cxlix) (S)-3-(1-hydroxypropan-2-yl)-8-(piperidin-1-yl)-6-(6-(trifluoromethyl)pyridin-3-yl)pyrido[3,4-d]pyrimidin-4(3H)-one
(cl)(S)-3-(1-hydroxypropan-2-yl)-8-(pyrrolidin-1-yl)-6-(6-(trifluoromethyl)pyridin-3-yl)pyrido[3,4-d]pyrimidin-4(3H)-one
(cli) (S)-6-(4-chlorophenyl)-3-(1-hydroxypropan-2-yl)-8-(piperidin-1-yl)pyrido[3,4-d]pyrimidin-4(3H)-one
(clii) (S)-3-(1-hydroxypropan-2-yl)-8-(pyridin-2-yl)-6-(6-(trifluoromethyl)pyridin-3-yl)pyrido[3,4-d]pyrimidin-4(3H)-one
(cliii) (S)-6-cyclohexyl-3-(1-hydroxypropan-2-yl)-8-(pyridin-3-yl)pyrido[3,4-d]pyrimidin-4(3H)-one
(cliv) (S)-3-(1-hydroxypropan-2-yl)-6-(pyridin-2-yl)-8-(pyridin-3-yl)pyrido[3,4-d]pyrimidin-4(3H)-one
(clv) (S)-3-(1-hydroxypropan-2-yl)-6-(2-methylthiazol-4-yl)-8-(pyridin-3-yl)pyrido[3,4-d]pyrimidin-4(3H)-one
(clvi) (S)-3-(1-hydroxypropan-2-yl)-6-(1-methyl-1H-1,2,3-triazol-5-yl)-8-(pyridin-3-yl)pyrido[3,4-d]pyrimidin-4(3H)-one
(clvii) (R)-6-(4-chlorophenyl)-8-(pyridin-3-yl)-3-(3,3,3-trifluoro-2-hydroxypropyl)pyrido[3,4-d]pyrimidin-4(3H)-one
(clviii) (S)-6-(4-chlorophenyl)-8-(pyridin-3-yl)-3-(3,3,3-trifluoro-2-hydroxypropyl)pyrido[3,4-d]pyrimidin-4(3H)-one.

### EXAMPLES

The synthesis of AhR antagonists of Table 2, along with biological data for these compounds, is described in PCT International Publication No. WO 2021/102288 A1, the contents of which are incorporated by reference herein in their entirety.

### Example 1: AhR Ligands Predictive of ICI Responsiveness

An experimental cohort of late stage (III-IV) non-small cell lung cancer patients (donors) were recruited to participate in a study evaluating the role of the microbiome on response to ICI therapies. Evaluated therapy types included any singular or combination use of therapies targeting PD-1, PD-L1 and CLTA-4. Samples collected to characterize the effect of the microbiome on patient response consisted of paired stool and plasma and were collected at two time points, (i) prior to the initiation of the therapy, and (ii) approximately six weeks following the initiation of the therapy. Patients were monitored for up to three years post-recruitment to establish response to the administered immune modulating therapies based on the adopted RECIST 1.1 criteria. A total of 73 patients qualified for this study and provided samples.

Fecal (intestinal luminal) and plasma samples collected from all donors at all timepoints were processed in parallel to characterize the signals from metabolites shared across the microbiome and host. Samples were stored according to best practices in the microbiome sciences.

Metabolite concentrations were measured by Metabolon, Inc., Durham, North Carolina, USA, using a targeted GC/MS, LC/MS and LC/MS/MS platform described in Zierer et al. (Nat Genet., 2018, 50(6):790-795) including Ultrahigh Performance Liquid Chromatography-Tandem Mass Spectroscopy (UPLC-MS/MS). The following instruments/conditions were used: Waters ACQUITY ultra-performance liquid chromatography (UPLC) and Thermo Scientific Q-Exactive high resolution/accurate mass spectrometer interfaced with a heated electrospray ionization (HESI-II) source and Orbitrap mass analyzer operated at 35,000 mass resolution. The sample extracts were dried and then reconstituted in solvents compatible to each of the following four assayed aliquots. Each reconstitution solvent contains a series of standards at fixed concentrations to ensure injection and chromatographic consistency.

The first aliquot was analyzed using acidic positive ion conditions, chromatographically optimized for more hydrophilic compounds. The extract was eluted from a C18 column (Waters UPLC BEH C18-2.1×100 mm, 1.7 m) using gradient elution with water and methanol containing 0.05% perfluoropentanoic acid (PFPA) and 0.1% formic acid (FA). The second aliquot was also analyzed using acidic positive ion conditions, however it was chromatographically optimized for more hydrophobic compounds. In this assay, the extract was eluted from the same C18 column using gradient elution with methanol, acetonitrile, water, 0.05% PFPA, and 0.01% FA, and was operated at an overall higher organic content. The third aliquot was analyzed using basic negative ion optimized conditions using a separate dedicated C18 column. The basic extract was eluted from the column using a gradient of methanol and water with 6.5 mM ammonium bicarbonate at pH 8. The fourth aliquot was analyzed using negative ionization following elution from a HILIC column (Waters UPLC BEH Amide 2.1×150 mm, 1.7 m) using a gradient of water and acetonitrile with 10 mM ammonium formate, pH 10.8. The MS analysis alternated between MS and data-dependent MSn scans using dynamic exclusion. The scan range varied slightly between assays but covered 70-1000 m/z. Raw data files were archived and extracted by Metabolon Inc.

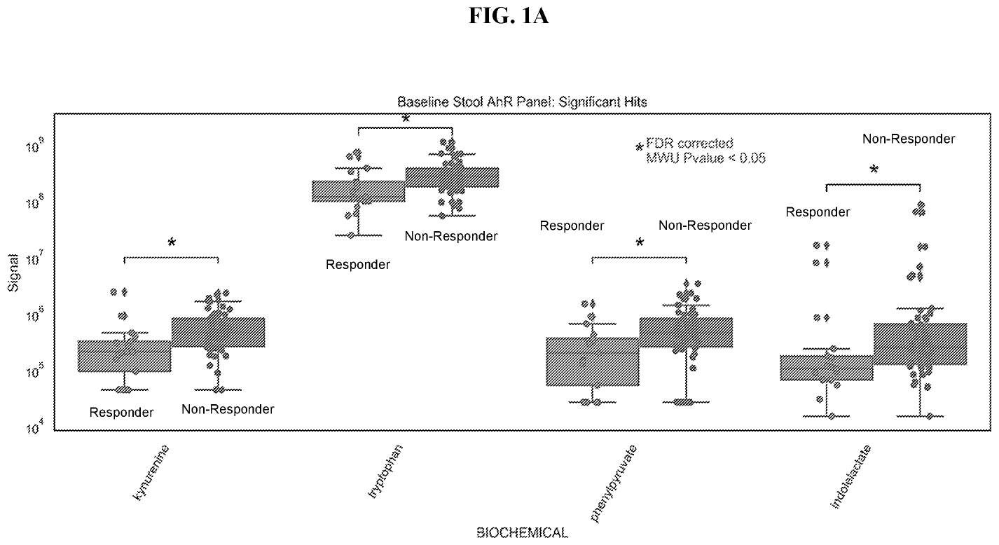

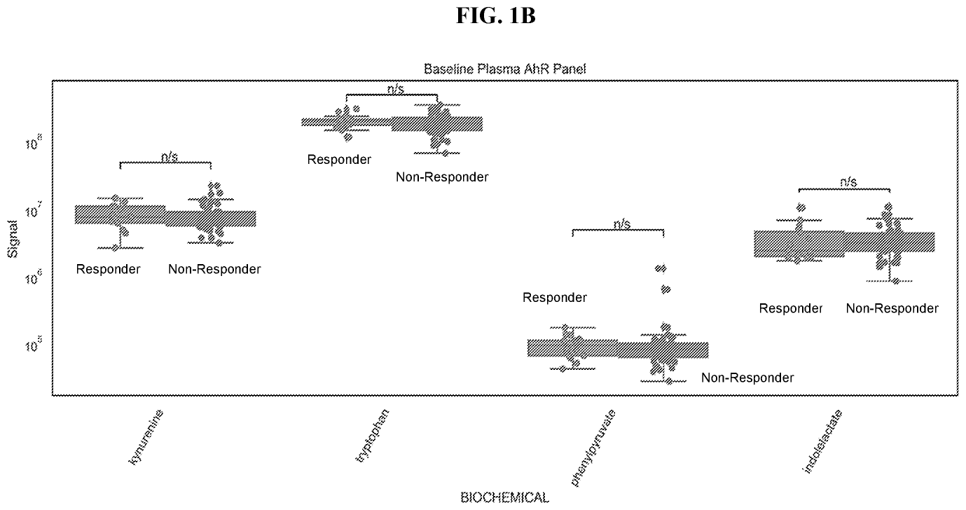

From the small molecule profiling data that was generated, a targeted panel of 29 compounds (Table 1) known to function as AhR ligands were investigated. Utilizing the metadata describing patient outcome, a binary representation of response to ICI therapy was constructed for each patient. By leveraging the baseline timepoint prior to actual initiation of therapy, the AhR ligands were further analyzed with a statistical association testing (MannWhitneyU with false discovery rate correction) in both the stool and plasma samples to assess any potential for such ligands to have a predictive or predetermining effect on the success of ICI therapy. As shown in FIG. 1A, four of the ligands, namely kynurenine, tryptophan, phenylpyruvate and indolelactate, were found to be differentially abundant at baseline with an FDR corrected p-value less than 0.05 when comparing between patients that eventually went on to become either responders or non-responders.

Of these four AhR ligands statistically associated with future response in this clinical study, all were found on average to be elevated in non-responders compared to the responders, suggesting the observation of depleted signals of these AhR ligands might be indicative of a higher probability of response to ICI therapies.

As shown in FIG. 1B, the corresponding signals for these ligands in the paired plasma samples from these donors showed no statistical difference using the same methodology, indicating that the elevation of these AhR ligands in patients that eventually went on to be non-responders is observable only through the direct measurement of the stool sample but not the plasma sample.

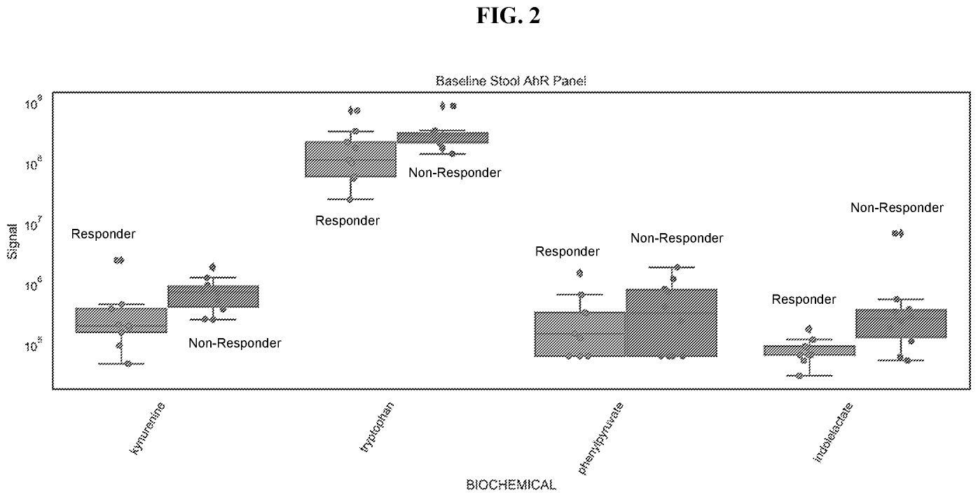

Although only a minority (19 of 73) of the clinical population, those patients that received ICI only with no other concomitant therapy, such as chemotherapy, also demonstrated this striking shift in elevation for these four AhR ligands, indicating future non-response to ICI therapies (FIG. 2). The data further demonstrates the relevance of microbial metabolism of these AhR ligands for predicting successful therapeutic response from ICI therapies.

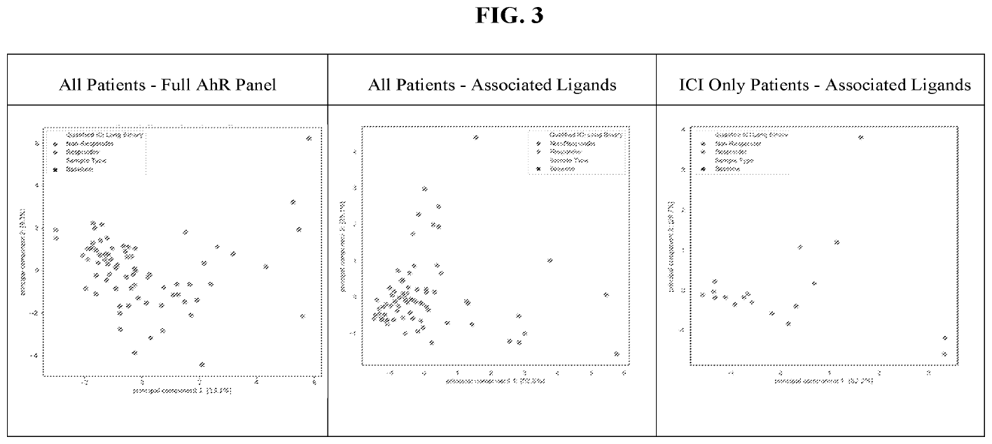

The signals from the full panel of 29 AhR ligands compared to the four statistically associated ligands allowed for a visual overview of these signals of future responders or non-responders as displayed through a principal component analysis for dimensionality reduction. As shown in the left and middle panels of FIG. 3, the grouping of responders qualitatively increased as one moved from the full panel to visualizing signals from only the four associated ligands. When comparing this view of the associated ligands in the ICI only donors, the resolution between responders and non-responders became much more striking (right panel), confirming the observation of these four AhR ligands as being potentially either predictive or deterministic in successful ICI therapies.

### Example 2: DRE-Luciferase Reporter Assay

AhR binds to dioxin responsive elements (DRE) upstream of the genes that it activates. One measure of AhR activity is activation of a reporter gene, such as luciferase, downstream of one or multiple DRE elements. Luciferase activity reflects activation and inhibition of AhR in the cells expressing this reporter. 20000 Human HepG2 liver carcinoma—AhR-Lucia reporter cells or Human HT29 colon adenocarcinoma-AhR reporter cells or other cell lines with a DRE-luciferase reporter stably transfected were plated in Eagle's Minimal Essential Medium, 10% heat-inactivated FBS, 1× non-essential amino acids Pen-Strep (10,000 U/mL) and Normocin (100 ug/mL) in plates (96-well, 384-well or other plates) and incubated overnight at 37° C. in a CO₂ incubator and treated with and without AhR antagonists at a log dilution starting at 100 μM.

One hour after the cells were plated, AhR activating ligands, such as TCDD, kynurenine, ITE (2-(1H-indole-3-ylcarbonyl)-4-thiazolecarboxylic methyl ester), VAF347, BNF (beta-naphthoflavone), FICZ (6-formylindolo(3,2-b) carbazole), or other AhR ligands at their specific EC₅₀ concentration, were added to the cells with or without an AhR antagonist.

Cells were incubated for 24 or 48 hours or another time point and the supernatant was analyzed for determination of luciferase activity as a read-out of the AhR activation or inhibition. Luciferase was measured with the commercial kit QUANTI-Luc™ assay solution kit from InvivoGen following the manufacturer's instructions.

The level of luciferase with only agonist ligand added was the maximum signal while the luciferase with no antagonist was the minimum signal. IC₅₀ values were determined as the concentration which inhibits half of the luciferase activity. The IC₅₀ level of luciferase of the novel AhR antagonists of the disclosure is reported in Table 3. “A” indicates an IC₅₀ value less than 100 nM, “B” indicates an IC₅₀ between 100 and 500 nM, “C” indicates an IC₅₀ above 500 nM, and “D” indicates that an IC₅₀ value could not be generated from the data.

TABLE 3

| | HepG2-Luc |
|---|---|
| Compound No. | IC₅₀ (nM) |
| 1 | A |
| 2 | B |
| 3 | B |
| 4 | B |
| 5 | A |
| 6 | D |
| 7 | A |
| 8 | A |
| 9 | A |
| 10 | B |
| 11 | C |
| 12 | A |
| 13 | A |
| 14 | A |
| 15 | A |
| 16 | A |
| 17 | A |
| 18 | A |
| 19 | C |
| 20 | B |
| 21 | A |
| 22 | A |
| 23 | A |
| 24 | A |
| 25 | A |
| 26 | B |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | A |
| 39 | A |
| 40 | C |
| 41 | A |
| 42 | A |
| 43 | D |
| 44 | A |
| 45 | A |
| 46 | A |
| 47 | A |
| 48 | A |
| 49 | A |
| 50 | A |
| 51 | A |
| 52 | C |
| 53 | B |
| 54 | A |
| 55 | A |
| 56 | B |
| 57 | A |
| 58 | A |
| 59 | C |
| 60 | A |
| 61 | B |
| 62 | A |
| 63 | B |
| 64 | C |
| 65 | B |
| 66 | A |
| 67 | A |
| 68 | A |
| 69 | B |
| 70 | A |
| 71 | A |
| 72 | A |
| 73 | C |
| 74 | A |
| 75 | B |
| 76 | B |
| 77 | C |
| 78 | B |
| 79 | A |
| 80 | C |
| 81 | A |
| 82 | A |
| 83 | A |
| 84 | C |
| 85 | A |
| 86 | A |
| 87 | A |
| 88 | A |
| 89 | A |
| 90 | A |
| 91 | B |
| 92 | A |
| 93 | B |
| 94 | A |
| 95 | A |
| 96 | A |
| 97 | C |
| 98 | A |
| 99 | A |
| 100 | C |
| 101 | A |
| 102 | A |
| 103 | A |
| 104 | A |
| 105 | A |
| 106 | A |
| 107 | A |
| 108 | A |
| 109 | A |
| 110 | A |
| 111 | A |
| 112 | A |
| 113 | A |
| 114 | A |
| 115 | A |
| 116 | A |
| 117 | A |
| 118 | A |
| 119 | A |
| 120 | A |
| 121 | A |
| 122 | A |
| 1123 | A |
| 124 | A |
| 125 | A |
| 126 | A |
| 127 | A |
| 128 | A |
| 129 | A |
| 130 | A |
| 131 | A |
| 132 | A |
| 133 | A |
| 134 | A |
| 135 | A |
| 136 | A |
| 137 | A |
| 138 | A |
| 139 | A |
| 140 | A |
| 141 | A |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | C |
| 156 | A |
| 157 | A |
| 158 | B |
| 159 | A |
| 160 | A |
| 161 | A |
| 162 | A |
| 163 | A |
| 164 | B |
| 165 | A |
| 166 | A |
| 167 | B |
| 168 | A |
| 169 | A |
| 170 | A |
| 171 | A |
| 172 | A |
| 173 | B |
| 174 | A |
| 175 | A |
| 176 | A |
| 177 | A |
| 178 | A |
| 179 | A |
| 180 | B |
| 181 | C |
| 182 | B |
| 183 | C |
| 184 | C |
| 185 enantiomer 1 | A |
| 185 enantiomer 2 | A |
| 186 | A |
| 187 | C |
| 188 | B |
| 189 | C |
| 190 | C |
| 191 | A |
| 192 | C |
| 193 | |

### Example 3: Combination Therapy

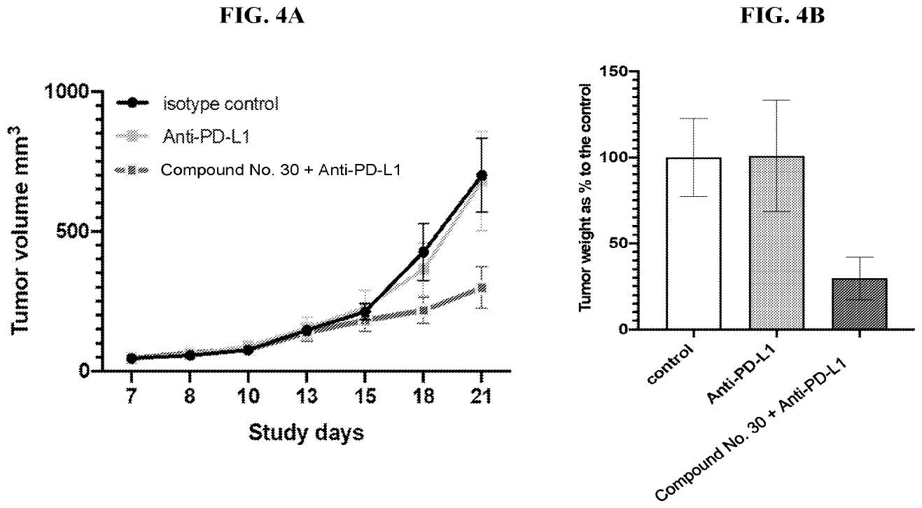

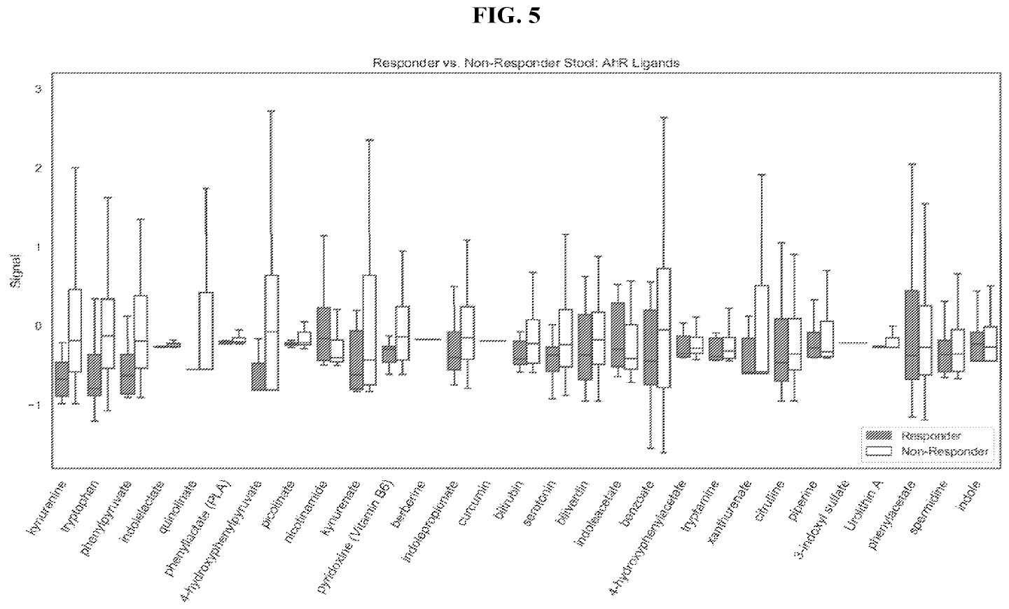

The anti-tumor efficacy of Compound No. 30 was studied in a CT26 mouse model which is unresponsive to anti-PD-L1 therapy only. CT26 cells were implanted subcutaneously into Balb/c mice which were then randomized and treated either with an isotope control antibody, an anti-PD-L1 antibody, or Compound No. 30 of Table 2 in combination with the anti-PD-L1 antibody. Compound No. 30 was dosed 10 mg/kg, p.o. once a day over 14 days and the anti-PD-L1 antibody was dosed at 10 mg/kg i.p. every 3 days. The tumor growth curves and tumor weight of all treatment groups are shown in FIGS. 4A and 4B. While the anti-PD-L1 antibody alone did not noticeably affect the tumor growth or tumor weight, coadministration of Compound No. 30 with the anti-PD-L1 antibody resulted in a reduction in tumor volume p=0.039 (FIG. 4A, Mann-Whitney, non-parametric test). In addition, coadministration of Compound No. 30 with the anti-PD-L1 antibody resulted in a reduction in tumor weight p=0.067 (FIG. 4B, Mann-Whitney, non-parametric test).

While the disclosure has been particularly shown and described with reference to specific embodiments, it should be understood by those having skill in the art that various changes in form and detail may be made therein without departing from the spirit and scope of the present disclosure.

What is claimed is:

1. A method of treating cancer in a patient in need thereof, comprising:
(a) measuring or having measured an amount of at least one aryl hydrocarbon receptor (AhR) ligand from a fecal sample obtained from the patient,
(b) comparing or having compared the amount of the at least one AhR ligand to a threshold score,
(c) determining or having determined that the patient would benefit from being treated with an AhR antagonist if the amount of the at least one AhR ligand is above the threshold score, and
(d) treating the patient determined to benefit in (c) with an effective amount of the AhR antagonist.

2. The method of claim 1, wherein the patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

3. The method of claim 1 or claim 2, wherein the patient has been treated with an immune-checkpoint inhibitor (ICI) therapy but is not responding to the ICI therapy.

4. The method of claim 1 or claim 2, wherein the patient has been treated with an ICI therapy and has developed resistance to the ICI therapy.

5. The method of any one of claims 1-4, wherein the at least one AhR ligand comprises kynurenine, tryptophan, phenylpyruvate, indolelactate, or combinations thereof.

6. The method of claim 5, wherein the at least one AhR ligand comprises kynurenine, tryptophan, phenylpyruvate, and indolelactate.

7. The method of any one of claims 1-6, wherein the amount of the at least one AhR ligand in the sample is measured by liquid chromatography mass spectrometry or gas-phase chromatography mass spectrometry.

8. The method of any one of claims 1-7, wherein the threshold score is determined by measuring the amount of the corresponding at least one AhR ligand in a control fecal sample obtained from a patient who is responding to an ICI therapy.

9. The method of any one of claims 1-8, wherein the AhR antagonist is a compound of Formula I: 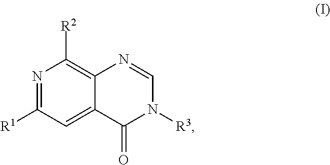
or a pharmaceutically acceptable salt thereof,
and wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines, and optionally substituted heterocycloalkyls, and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

10. The method of any one of claims 1-9, wherein the AhR antagonist is a compound of Formula Ia: 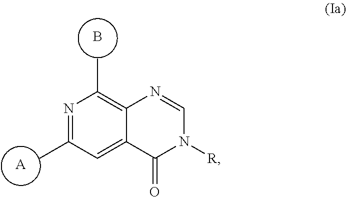
or a pharmaceutically acceptable salt thereof,
and wherein:
ring A is chosen from optionally substituted aryls, optionally substituted heteroaryls, optionally substituted cycloalkyls, and optionally substituted heterocycloalkyls,
ring B is chosen from optionally substituted aryls, optionally substituted heteroaryls, optionally substituted cycloalkyls, and optionally substituted heterocycloalkyls, and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

11. The method of any one of claims 1-10, wherein the AhR antagonist is any one listed in Table 2, or a pharmaceutically acceptable salt thereof.

12. An in vitro method of determining whether a patient would benefit from being treated with an aryl hydrocarbon receptor (AhR) antagonist, comprising:
(a) measuring or having measured an amount of at least one AhR ligand from a fecal sample obtained from the patient,
(b) comparing or having compared the amount of the at least one AhR ligand to a threshold score, and
(c) determining that the patient would benefit from being treated with an AhR antagonist if the amount of the at least one AhR ligand is above the threshold score.

13. The method of claim 12, wherein the patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

14. The method of claim 12 or claim 13, wherein the patient has been treated with an immune-checkpoint inhibitor (ICI) therapy but is not responding to the ICI therapy.

15. The method of claim 12 or claim 13, wherein the patient has been treated with an ICI therapy and has developed resistance to the ICI therapy.

16. The method of any one of claims 12-15, wherein the at least one AhR ligand comprises kynurenine, tryptophan, phenylpyruvate, indolelactate, or combinations thereof.

17. The method of claim 16, wherein the at least one AhR ligand comprises kynurenine, tryptophan, phenylpyruvate, and indolelactate.

18. The method of any one of claims 12-17, wherein the amount of the at least one AhR ligand in the sample is measured by liquid chromatography mass spectrometry or gas-phase chromatography mass spectrometry.

19. The method of any one of claims 12-18, wherein the threshold score is determined by measuring the amount of the corresponding at least one AhR ligand in a control fecal sample obtained from a patient who is responding to an ICI therapy.

20. The method of any one of claims 11-19, wherein the AhR antagonist is a compound of Formula I: 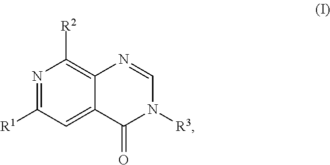
or a pharmaceutically acceptable salt thereof,
and wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines, and optionally substituted heterocycloalkyls, and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

21. The method of any one of claims 12-20, wherein the AhR antagonist is a compound of Formula Ia: 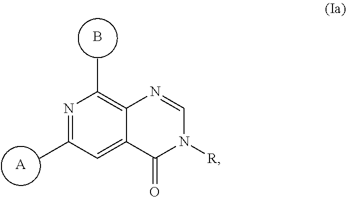
or a pharmaceutically acceptable salt thereof,
and wherein:
ring A is chosen from optionally substituted aryls, optionally substituted heteroaryls, optionally substituted cycloalkyls, and optionally substituted heterocycloalkyls,
ring B is chosen from optionally substituted aryls, optionally substituted heteroaryls, optionally substituted cycloalkyls, and optionally substituted heterocycloalkyls, and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

22. The method of any one of claims 12-20, wherein the AhR antagonist is any one listed in Table 2, or a pharmaceutically acceptable salt thereof.

23. A method of treating cancer in a patient in need thereof with a combination therapy comprising an immune checkpoint inhibitor (ICI) and an aryl hydrocarbon receptor (AhR) antagonist, comprising:
(a) measuring or having measured an amount of at least one AhR ligand from a fecal sample obtained from the patient,
(b) comparing or having compared the amount of the at least one AhR ligand to a threshold score,
(c) determining or having determined that the patient would benefit from the combination therapy if the amount of the at least one AhR ligand is above the threshold score, and
(d) treating the patient determined to benefit in (c) with the combination therapy.

24. The method of claim 23, wherein the patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

25. The method of claim 23 or claim 24, wherein the patient has been treated with an ICI therapy but is not responding to the ICI therapy.

26. The method of claim 23 or claim 24, wherein the patient has been treated with an ICI therapy and has developed resistance to the ICI therapy.

27. The method of any one of claims 23-26, wherein the at least one AhR ligand comprises kynurenine, tryptophan, phenylpyruvate, indolelactate, or combinations thereof.

28. The method of claim 27, wherein the at least one AhR ligand comprises kynurenine, tryptophan, phenylpyruvate, and indolelactate.

29. The method of any one of claims 23-28, wherein the amount of the at least one AhR ligand in the sample is measured by liquid chromatography mass spectrometry or gas-phase chromatography mass spectrometry.

30. The method of any one of claims 23-29, wherein the ICI therapy comprises an anti-PD-1 antibody, an anti-PD-L1 antibody, an anti-CTLA-4 antibody, an anti-LAG-3 antibody, an anti-TIM-3 antibody, an anti-TIGIT antibody, an anti-VISTA antibody, or combinations thereof.

31. The method of any one of claims 23-30, wherein the threshold score is determined by measuring the amount of the corresponding at least one AhR ligand in a control fecal sample obtained from a patient who is responding to an ICI therapy.

32. The method of any one of claims 23-31, wherein the AhR antagonist is a compound of Formula I: 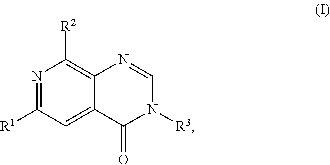
or a pharmaceutically acceptable salt thereof,
and wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines, and optionally substituted heterocycloalkyls, and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

33. The method of any one of claims 23-32, wherein the AhR antagonist is a compound of Formula Ia: 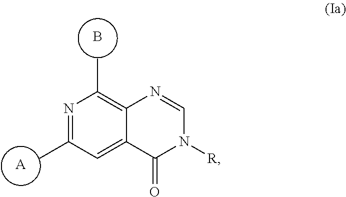
or a pharmaceutically acceptable salt thereof,
and wherein:
ring A is chosen from optionally substituted aryls, optionally substituted heteroaryls, optionally substituted cycloalkyls, and optionally substituted heterocycloalkyls,
ring B is chosen from optionally substituted aryls, optionally substituted heteroaryls, optionally substituted cycloalkyls, and optionally substituted heterocycloalkyls, and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

34. The method of any one of claims 23-33, wherein the AhR antagonist is any one listed in Table 2, or a pharmaceutically acceptable salt thereof.

35. An in vitro method of predicting a patient's cancer responsiveness to an immune checkpoint inhibitor (ICI) therapy, comprising:
(a) measuring or having measured an amount of at least one aryl hydrocarbon receptor (AhR) ligand from a fecal sample obtained from the patient,
(b) comparing or having compared the amount of the at least one AhR ligand to a threshold score, and
(c) determining that the patient's cancer is likely to be responsive to an ICI therapy if the amount of the at least one AhR ligand is equal to or below the threshold score.

36. The method of claim 35, wherein the patient suffers from non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, triple-negative breast cancer, esophageal neoplasms, multiple myeloma, gastric and gastroesophageal junction cancer, melanoma, Hodgkin lymphoma, hepatocellular carcinoma, lung cancer, head and neck cancer, non-Hodgkin lymphoma, metastatic clear cell renal carcinoma, squamous cell lung carcinoma, mesothelioma, gastric cancer, gastroesophageal junction cancer, metastatic melanoma, metastatic non-cutaneous melanoma, urothelial cancer, diffuse large B-cell lymphoma, renal cell cancer, ovarian cancer, fallopian tube cancer, peritoneal neoplasms, extensive stage small cell lung cancer, bladder cancer, transitional cell carcinoma, prostatic neoplasms, recurrent or metastatic PD-L1 positive or negative squamous cell carcinoma of the head and neck, recurrent squamous cell lung cancer, advanced solid malignancies, hypo pharyngeal squamous cell carcinoma, laryngeal squamous cell carcinoma, unresectable or metastatic melanoma, biliary tract neoplasms, esophageal squamous cell carcinoma, breast cancer, pancreatic cancer, glioblastoma, metastatic cancer, prostatic cancer, solid organ cancer, stomach cancer, colon cancer, or liver cancer.

37. The method of claim 35 or claim 36, wherein the at least one AhR ligand comprises kynurenine, tryptophan, phenylpyruvate, indolelactate, or combinations thereof.

38. The method of claim 37, wherein the at least one AhR ligand comprises kynurenine, tryptophan, phenylpyruvate, and indolelactate.

39. The method of any one of claims 35-38, wherein the amount of the at least one AhR ligand in the sample is measured by liquid chromatography mass spectrometry or gas-phase chromatography mass spectrometry.

40. The method of any one of claims 35-39, wherein the ICI therapy comprises an anti-PD-1 antibody, an anti-PD-L1 antibody, an anti-CTLA-4 antibody, an anti-LAG-3 antibody, an anti-TIM-3 antibody, an anti-TIGIT antibody, an anti-VISTA antibody, or combinations thereof.

41. The method of any one of claims 35-40, wherein the threshold score is determined by measuring the amount of the corresponding at least one AhR ligand in a control fecal sample obtained from a patient who is responding to an ICI therapy.

42. The method of any one of claims 35-41, further comprising determining that the patient's cancer is likely to be responsive to an ICI therapy in combination with an AhR antagonist if the amount of the at least one AhR ligand is above the threshold score.

43. The method of claim 42, wherein the AhR antagonist is a compound of Formula I: 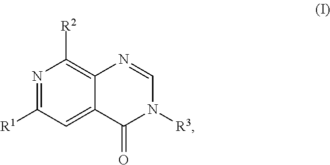
or a pharmaceutically acceptable salt thereof,
and wherein:
each of R¹ and R² is independently chosen from optionally substituted alkyls, optionally substituted esters, optionally substituted heteroalkyls, optionally substituted acyls, optionally substituted amides, optionally substituted aryls, optionally substituted heteroaryls, optionally substituted cycloalkyls, optionally substituted amines, and optionally substituted heterocycloalkyls, and
R³ is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, optionally substituted amines, cyano, halos, hydroxy, and —C(O)H.

44. The method of claim 42 or claim 43, wherein the AhR antagonist is a compound of Formula Ia: 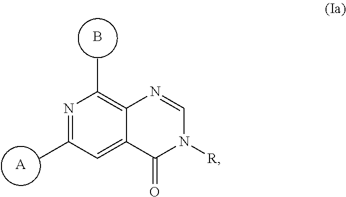
or a pharmaceutically acceptable salt thereof,
and wherein:
ring A is chosen from optionally substituted aryls, optionally substituted heteroaryls, optionally substituted cycloalkyls, and optionally substituted heterocycloalkyls,
ring B is chosen from optionally substituted aryls, optionally substituted heteroaryls, optionally substituted cycloalkyls, and optionally substituted heterocycloalkyls, and
R is chosen from hydrogen, optionally substituted alkyls, optionally substituted acyls, optionally substituted amides, optionally substituted aryls, optionally substituted cycloalkyls, optionally substituted esters, optionally substituted heteroalkyls, optionally substituted heteroaryls, optionally substituted heterocycloalkyls, amino, cyano, halos, hydroxy, and —C(O)H.

45. The method of any one of claims 42-44, wherein the AhR antagonist is any one listed in Table 2, or a pharmaceutically acceptable salt thereof.
